(19) United States
(12) Patent Application Publication
(10) Pub. No.: US 2025/0082926 A1
(43) Pub. Date: Mar. 13, 2025

(54) IMPLANTABLE STIMULATING ASSEMBLY

(71) Applicant: Cochlear Limited, Macquarie University (AU)

(72) Inventors: Peter Raymond Sibary, Luddenham (AU); Nicholas Charles Pawsey, North Ryde (AU)

(21) Appl. No.: 18/958,335

(22) Filed: Nov. 25, 2024

Related U.S. Application Data
(63) Continuation-in-part of application No. 17/181,605, filed on Feb. 22, 2021, now abandoned.
Application No. 17/181,605 is a division of application No. 15/249,799, filed on Aug. 29, 2016, now Pat. No. 10,926,081.
(60) Provisional application No. 62/211,434, filed on Aug. 28, 2015.

(51) Int. Cl. A61N 1/05; A61N 1/36; A61N 1/378

(52) U.S. Cl. CPC A61N 1/0541; A61N 1/3787; A61N 1/36038

(57) ABSTRACT

An elongate stimulation assembly of an implantable stimulation device, including a first portion including a plurality of electrodes, lead wires extending from the first portion in electrical communication with the plurality of electrodes, the lead wires being located in an elongate lead body and a cage component extending in an elongate manner at least partially along with the lead wires, wherein the lead wires extend within the cage component.

### CROSS-REFERENCE TO RELATED APPLICATIONS

This application is a continuation in part of U.S. patent application Ser. No. 17/181,605, filed on Feb. 22, 2021, which is a Divisional application of U.S. patent application Ser. No. 15/249,799, filed Aug. 29, 2016, which claims priority to Provisional U.S. Patent Application No. 62/211,434, entitled IMPLANTABLE STIMULATING ASSEMBLY, filed on Aug. 28, 2015, naming Peter Raymond Sibary of Macquarie University, Australia as an inventor, the entire contents of all of these applications being incorporated herein by reference in its entirety.

### BACKGROUND

Hearing loss, which may be due to many different causes, is generally of two types: conductive and sensorineural. Sensorineural hearing loss is due to the absence or destruction of the hair cells in the cochlea that transduce sound signals into nerve impulses. Various hearing prostheses are commercially available to provide individuals suffering from sensorineural hearing loss with the ability to perceive sound. One example of a hearing prosthesis is a cochlear implant.

Conductive hearing loss occurs when the normal mechanical pathways that provide sound to hair cells in the cochlea are impeded, for example, by damage to the ossicular chain or the ear canal. Individuals suffering from conductive hearing loss may retain some form of residual hearing because the hair cells in the cochlea may remain undamaged.

Individuals suffering from conductive hearing loss typically receive an acoustic hearing aid. Hearing aids rely on principles of air conduction to transmit acoustic signals to the cochlea. In particular, a hearing aid typically uses an arrangement positioned in the recipient's ear canal or on the outer ear to amplify a sound received by the outer ear of the recipient. This amplified sound reaches the cochlea causing motion of the perilymph and stimulation of the auditory nerve.

In contrast to hearing aids, which rely primarily on the principles of air conduction, certain types of hearing prostheses commonly referred to as cochlear implants convert a received sound into electrical stimulation. The electrical stimulation is applied to the cochlea, which results in the perception of the received sound.

### SUMMARY

An elongate stimulation assembly of an implantable stimulation device, comprising an intra-cavity portion including a plurality of electrodes and an extra-cavity portion extending from the intra-cavity portion, wherein the extra-cavity portion includes a plurality of electrical lead wires in electrical communication with the plurality of electrodes and a malleable component extending in an elongate manner such that at least a portion of the malleable component is located further away from or the same distance from a longitudinal axis of the extra-cavity portion than a portion of least one of the electrical leads of the plurality of electrical leads.

An elongate stimulation assembly of an implantable stimulation device, comprising a first portion including a plurality of electrodes, lead wires extending from the first portion in electrical communication with the plurality of electrodes, the lead wires being located in an elongate lead body and a cage component extending in an elongate manner at least partially along with the lead wires, wherein the lead wires extend within the cage component.

A device, comprising a stimulating assembly of an implantable stimulating device, including a lead assembly made at least partially of a structure having portions that are more susceptible to plastic deformation than other portions of the structure, wherein the device is configured to resist movement of at least a portion of the lead assembly, the movement of the lead assembly being due to the plastic deformation.

A method, comprising obtaining an implantable cage apparatus, moulding silicone so that the cage apparatus is encapsulated in an overmoulded silicone tube, thus resulting in a silicone tube-cage assembly and inserting the silicone tube-cage assembly over a plurality of lead wires of a cochlear implant electrode array and/or inserting the plurality of lead wires into the silicone tube-cage assembly.

### BRIEF DESCRIPTION OF THE DRAWINGS

Embodiments of the present invention are described below with reference to the attached drawings, in which:

FIG. 1 is a perspective view of an exemplary hearing prosthesis utilized in some exemplary embodiments;

FIG. 2 is a side view of the implantable components of the cochlear implant illustrated in FIG. 1;

FIG. 3 is a side view of an embodiment of the electrode array illustrated in FIGS. 1 and 2 in a curled orientation;

FIG. 4 is a functional schematic of an electrode array including 22 electrodes spaced apparat from one another;

FIG. 5 is a schematic of an apparatus to which some teachings detailed herein are applicable;

FIG. 6 is a schematic diagram conceptually illustrating another exemplary prosthesis to with the teachings herein are applicable in some embodiments;

FIG. 7 is a side-view of a portion of the anatomy of a human along with an exemplary embodiment;

FIG. 8 is a schematic of an apparatus to which some teachings detailed herein have been applied;

FIG. 9 is a quasi-functional side view of a portion of the embodiment of FIG. 8;

FIG. 10 is a cross-sectional view of the embodiment of FIG. 9;

FIG. 11A is a schematic of another apparatus to which some teachings detailed herein have been applied;

FIG. 11B is a schematic of another apparatus to which some teachings detailed herein have been applied;

FIG. 12 is a schematic of another apparatus to which some teachings detailed herein have been applied;

FIG. 13 is a schematic of a portion of FIG. 12;

FIG. 14 is a side view of the apparatus of FIG. 12;

FIG. 15 is a cross-sectional view of the embodiment of FIG. 12;

FIG. 16 is a schematic of another apparatus to which some teachings detailed herein have been applied;

FIG. 17 is a side-view of a portion of the anatomy of a human along with another exemplary embodiment;

FIG. 18 is a top-view of a portion of the anatomy of a human along with another exemplary embodiment;

FIGS. 19-22 are side-views of a portion of the anatomy of a human along with other exemplary embodiments;

FIG. 23 is a flowchart according to an exemplary method;

FIG. 24 is a flowchart according to another exemplary method;

FIG. 25 is a top-view of a portion of the anatomy of a human along with another exemplary embodiment;

FIG. 26 is a side view of a portion of the anatomy of a human along with another exemplary embodiment;

FIG. 27 is a quasi-functional side view of a portion of the an alternate embodiment of the embodiment of FIG. 8;

FIG. 28 is a cross-sectional view of the embodiment of FIG. 27;

FIG. 29 is a cross-sectional view of an alternate embodiment of the embodiment of FIG. 8;

FIG. 30 is a cross-sectional view of another alternate embodiment of the embodiment of FIG. 8;

FIG. 31 is a cross-sectional view of another alternate embodiment of the embodiment of FIG. 8;

FIG. 32 is a cross-sectional view of another alternate embodiment of the embodiment of FIG. 8;

FIG. 33 is a cross-sectional view of another alternate embodiment of the embodiment of FIG. 8;

FIG. 34 is a cross-sectional view of another alternate embodiment of the embodiment of FIG. 8;

FIG. 35 is a cross-sectional view of another alternate embodiment of the embodiment of FIG. 8;

FIG. 36 is a cross-sectional view of another alternate embodiment of the embodiment of FIG. 8;

FIG. 37 is a cross-sectional view of another alternate embodiment of the embodiment of FIG. 8;

FIG. 38 is a quasi-functional side view of a portion of another embodiment of the embodiment of FIG. 6;

FIG. 39 is a cross-sectional view of the embodiment of FIG. 38;

FIG. 40 is a cross-sectional view of another alternate embodiment of the embodiment of FIG. 8;

FIG. 41 is a quasi-functional side view of a portion of another embodiment of the embodiment of FIG. 8;

FIG. 42 is a cross-sectional view of the embodiment of FIG. 38;

FIG. 43 is a cross-sectional view of an embodiment of a cage;

FIG. 44 is a cross-sectional view of an embodiment of a cage with one or more lead wires;

FIG. 45 is a cross-sectional view of an embodiment of a cage with two or more lead wires;

FIG. 46 is a cross-sectional view of an embodiment of a cage with two or more lead wires;

FIG. 47 is a cross-sectional view of an embodiment of a cage with one or more lead wires;

FIG. 48 is a cross-sectional view of an embodiment of a cage with three or more lead wires;

FIG. 49 is a cross-sectional view of an embodiment of a cage with two or more lead wires;

FIG. 50 is a cross-sectional view of an embodiment of a stimulating assembly;

FIG. 51 is a cross-sectional view of an embodiment of a cage with one or more lead wires;

FIGS. 52 and 53 are views of another embodiment;

FIG. 54 is a view of a cage structure;

FIGS. 55 and 56 are flowcharts for exemplary methods;

FIG. 57 depicts a schematic according to another exemplary embodiment;

FIG. 58 depicts a schematic depicting additional details of the embodiment of FIG. 57;

FIG. 59 depicts a schematic depicting a quasi-conceptual view of an exemplary embodiment;

FIGS. 60-73 depicts schematics depicting quasi-conceptual views of various exemplary embodiments;

FIG. 74 depicts an exemplary flow chart according to an exemplary method; and

FIG. 75A depicts a schematic according to another exemplary embodiment.

FIGS. 75B-80 depict schematics according to other exemplary embodiment.

### DETAILED DESCRIPTION

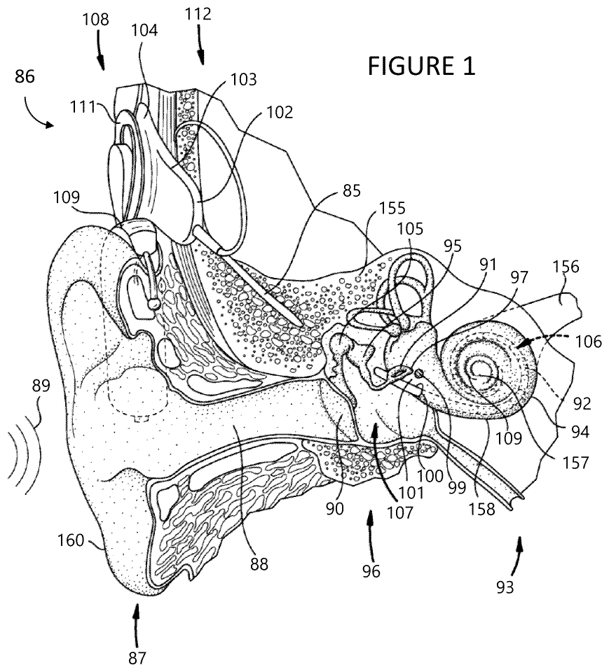

FIG. 1 is perspective view of a totally implantable cochlear implant according to an exemplary embodiment, referred to as cochlear implant 112, implanted in a recipient. The cochlear implant 112 is part of a system 86 that can include external components, as will be detailed below.

In an alternate embodiment, the cochlear implant system is not a totally implantable system. By way of example, the cochlear implant system includes an external component that includes a microphone and a sound processor. The sound processor processes signals from the microphone, and generates a signal that is transmitted transcutaneously to an implantable component which then uses the signal to stimulate tissue and evoke a hearing percept.

It is noted that in some conventional parlances, the entire system 86 is referred to as a cochlear implant, especially in the case of a cochlear implant that is not totally implantable. Herein, the phrase cochlear implant refers to the implantable component, and the phrase cochlear implant system refers to the entire system 86. That is, the phrase cochlear implant corresponds to the implantable component of a non-totally implantable cochlear implant system.

The recipient has an outer ear 87, a middle ear 96 and an inner ear 93. Components of outer ear 87, middle ear 96 and inner ear 93 are described below, followed by a description of cochlear implant 112.

In a fully functional ear, outer ear 87 comprises an auricle 160 and an ear canal 88. An acoustic pressure or sound wave 89 is collected by auricle 160 and channeled into and through ear canal 88. Disposed across the distal end of ear canal 88 is a tympanic membrane 90 which vibrates in response to sound wave 89. This vibration is coupled to oval window or fenestra ovalis 97 through three bones of middle ear 96, collectively referred to as the ossicles 107 and comprising the malleus 105, the incus 95 and the stapes 91. Bones 105, 95, and 91 of middle ear 96 serve to filter and amplify sound wave 89, causing oval window 97 to articulate, or vibrate in response to vibration of tympanic membrane 90. This vibration sets up waves of fluid motion of the perilymph within cochlea 94. Such fluid motion, in turn, activates tiny hair cells (not shown) inside of cochlea 94. Activation of the hair cells causes appropriate nerve impulses to be generated and transferred through the spiral ganglion cells (not shown) and auditory nerve 156 to the brain (also not shown) where they are perceived as sound.

As shown, cochlear implant 112 comprises one or more components which are temporarily or permanently implanted in the recipient. Cochlear implant 112 is shown in FIG. 1 along with an external device 108, that is part of system 86 (along with cochlear implant 112), which, as described below, is configured to provide power to the cochlear implant.

In the illustrative arrangement of FIG. 1, external device 108 may comprise a power source (not shown) disposed in a Behind-The-Ear (BTE) unit 109. External device 108 also includes components of a transcutaneous energy transfer link, referred to as an external energy transfer assembly. The transcutaneous energy transfer link is used to transfer power and/or data to cochlear implant 112. Various types of energy transfer, such as infrared (IR), electromagnetic, capacitive and inductive transfer, may be used to transfer the power and/or data from external device 108 to cochlear implant 112. In the illustrative embodiments of FIG. 1, the external energy transfer assembly comprises an external coil 111 that forms part of an inductive radio frequency (RF) communication link. External coil 111 is typically a wire antenna coil comprised of multiple turns of electrically insulated single-strand/or multi-strand platinum or gold wire. External device 108 also includes a magnet (not shown) positioned within the turns of wire of external coil 111. It should be appreciated that the external device shown in FIG. 1 is merely illustrative, and other external devices may be used with embodiments of the present invention.

Cochlear implant 112 comprises an internal energy transfer assembly 103 which may be positioned in a recess of the temporal bone adjacent auricle 160 of the recipient. As detailed below, internal energy transfer assembly 103 is a component of the transcutaneous energy transfer link and receives power and/or data from external device 108. In the illustrative embodiment, the energy transfer link comprises an inductive RF link, and internal energy transfer assembly 103 comprises a primary internal coil 104. Internal coil 104 is typically a wire antenna coil comprised of multiple turns of electrically insulated single-strand/or multi-strand platinum or gold wire.

Cochlear implant 112 further comprises a main implantable component 102 and an elongate stimulating assembly 85. In embodiments of the present invention, internal energy transfer assembly 103 and main implantable component 102 are hermetically sealed within a biocompatible housing. In embodiments of the present invention, main implantable component 102 includes a sound processing unit (not shown) to convert the sound signals received by the implantable microphone in internal energy transfer assembly 103 to data signals. Main implantable component 102 further includes a stimulator unit (also not shown) which generates electrical stimulation signals based on the data signals. The electrical stimulation signals are delivered to the recipient via elongate stimulating assembly 85.

Elongate stimulating assembly 85 has a proximal end connected to main implantable component 102, and a distal end implanted in cochlea 94. Stimulating assembly 85 extends from main implantable component 102 to cochlea 94 through mastoid bone 155. In some embodiments stimulating assembly 85 may be implanted at least in basal region 158, and sometimes further. For example, stimulating assembly 85 may extend towards apical end of cochlea 94, referred to as cochlea apex 157. In certain circumstances, stimulating assembly 85 may be inserted into cochlea 94 via a cochleostomy 100. In other circumstances, a cochleostomy may be formed through round window 99, oval window 97, the promontory 101 or through an apical turn 109 of cochlea 94.

Stimulating assembly 85 comprises a longitudinally aligned and distally extending array 106 of electrodes 92, disposed along a length thereof. As noted, a stimulator unit generates stimulation signals which are applied by stimulating contacts 92, which, in an exemplary embodiment, are electrodes, to cochlea 94, thereby stimulating auditory nerve 156. In an exemplary embodiment, stimulation contacts can be any type of component that stimulates the cochlea (e.g., mechanical components, such as piezoelectric devices that move or vibrate, thus stimulating the cochlea (e.g., by inducing movement of the fluid in the cochlea), electrodes that apply current to the cochlea, etc.). Embodiments detailed herein will generally be described in terms of a stimulating assembly 85 utilizing electrodes as elements 92. It is noted that alternate embodiments can utilize other types of stimulating devices. Any device, system or method of stimulating the cochlea can be utilized in at least some embodiments.

As noted, cochlear implant 112 comprises a totally implantable prosthesis that is capable of operating, at least for a period of time, without the need for external device 108. Therefore, cochlear implant 112 further comprises a rechargeable power source (not shown) that stores power received from external device 108. The power source may comprise, for example, a rechargeable battery. During operation of cochlear implant 112, the power stored by the power source is distributed to the various other implanted components as needed. The power source may be located in main implantable component 102 or disposed in a separate implanted location.

It is noted that the teachings detailed herein and/or variations thereof can be utilized with a non-totally implantable prosthesis. That is, in an alternate embodiment of the cochlear implant 112, the cochlear implant 112, and thus system 86, is a traditional hearing prosthesis.

While various aspects of the present invention are described with reference to a cochlear implant (whether it be a device utilizing electrodes or stimulating contacts that impart vibration and/or mechanical fluid movement within the cochlea), it will be understood that various aspects of the embodiments detailed herein are equally applicable to other stimulating medical devices having an array of electrical simulating electrodes such as auditory brain implant (ABI), functional electrical stimulation (FES), spinal cord stimulation (SCS), penetrating ABI electrodes (PABI), and so on. Further, it is noted that the teachings herein are applicable to stimulating medical devices having electrical stimulating electrodes of all types such as straight electrodes, peri-modiolar electrodes and short/basal electrodes. Also, various aspects of the embodiments detailed herein and/or variations thereof are applicable to devices that are non-stimulating and/or have functionality different from stimulating tissue, such as for, example, any intra-body dynamic phenomenon (e.g., pressure, or other phenomenon consistent with the teachings detailed herein) measurement/sensing, etc., which can include use of these teachings to sense or otherwise detect a phenomenon at a location other than the cochlea (e.g., within a cavity containing the brain, the heart, etc.). Additional embodiments are applicable to bone conduction devices, Direct Acoustic Cochlear Stimulators/Middle Ear Prostheses, and conventional acoustic hearing aids. Any device, system, or method of evoking a hearing percept can be used in conjunction with the teachings detailed herein. The teachings detailed herein are applicable to any device, system or method where an elongate lead having elastic properties or the like has utilitarian value with respect to positioning thereof.

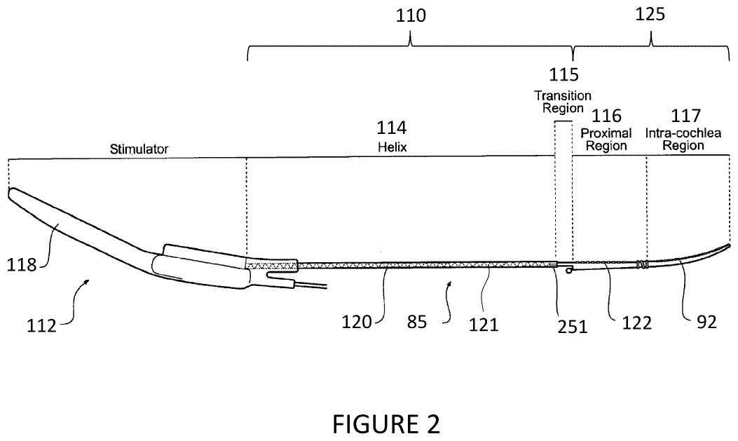

Still focusing on a cochlear implant, FIG. 2 is a side view of the cochlear implant 112 without the other components of system 86 (e.g., the external components). Cochlear implant 112 comprises a receiver/stimulator 118 (combination of main implantable component 102 and internal energy transfer assembly 103) and an elongate stimulating assembly 85. Stimulating assembly 85 includes a helix region 114 that includes a body 120 in which is embedded (e.g., in the case where the body is silicone or another biocompatible material molded around wire leads) or otherwise containing (e.g., in the case where the body is a conduit or tube) electrical lead wires 121 in a helix (more on this below), a transition region 115 (which can be part of the body 120), a proximal region 116, and an intra-cochlear region 117. The proximal region 116, in this embodiment, is connected to the transition region 115 via a distinct connection 251, although in other embodiments, the transition region is blended into the helix region 114 (and the proximal region 116). Proximal region 116 and intra-cochlear region 117 form an electrode array 125. The portion of the stimulating assembly 85 that extends from the receiver/stimulator 118 to the electrode array 125 is referred to herein as the lead assembly, indicated by reference numeral 110 in FIG. 2. In an exemplary embodiment, proximal region 116 is located in the middle-ear cavity of the recipient after implantation of the intra-cochlear region 117 into the cochlea. Thus, proximal region 116 corresponds to a middle-ear cavity sub-section of the stimulating assembly 85. In some exemplary embodiments, nubs 122 are provided on the outer surface of the proximal region to aid in the manipulation of the electrode array assembly 125 during insertion of the intra-cochlear region into the cochlea. Electrode array assembly 125, and in particular, intra-cochlear region 117 of electrode array assembly 125, supports a plurality of electrode contacts 92. These electrode contacts 92 are each connected to a respective conductive pathway, such as wires, PCB traces, etc. (not shown) which are connected to receiver/stimulator 118, through which respective stimulating electrical signals for each electrode contact 92 travel.

It is noted that in some embodiments, the helix region 114 does not extend as far as that depicted in FIG. 1, and the transition region 115 is thus longer. That is, in some exemplary embodiments, the helix region 114 does not extend substantially the full length between the receiver/stimulator 118 and the proximal region 116, but instead extends less than that (e.g., about half the distance), where the remaining distance is established by substantially straight lead wires, or at least wires that are not substantially helixed. Any arrangement of lead wires that can enable the teachings detailed herein and/or variations thereof to be practiced can be utilized in some exemplary embodiments.

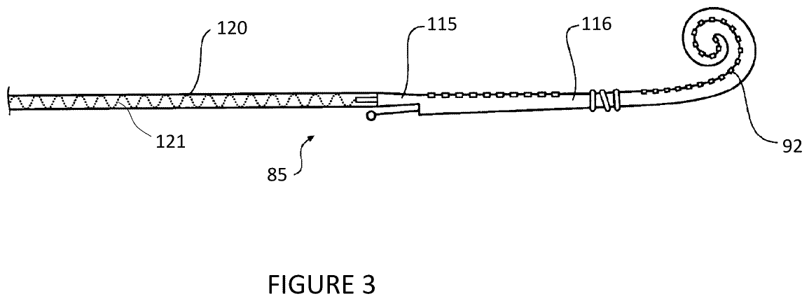

FIG. 3 is a side view of a portion of stimulating assembly 85 where the electrode array of the electrode array assembly 125 is in a curled orientation, as it would be when inserted in a recipient's cochlea, with electrode contacts 92 located on the inside of the curve. FIG. 3 depicts the electrode array of FIG. 2 in situ in a patient's cochlea 94.

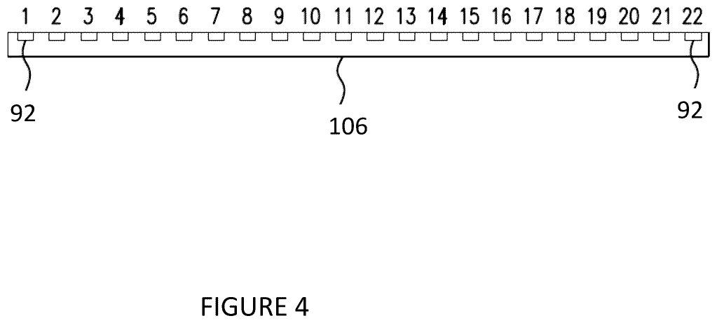

FIG. 4 illustrates a more detailed view, albeit functionally, of an exemplary electrode array 106 comprising a plurality of electrodes 92 labeled 1-22, in accordance with an embodiment. In an exemplary embodiment, each electrode 92 is an electrode that corresponds to a specific frequency band channel of the cochlear implant 112, where electrode 22 corresponds to the lowest frequency band (channel), and electrode 1 corresponds to the highest frequency band (channel). Briefly, it is noted that during stimulation by the electrodes to evoke a hearing percept, one or more electrodes 92 is activated at a given electrode stimulation level (e.g., current level).

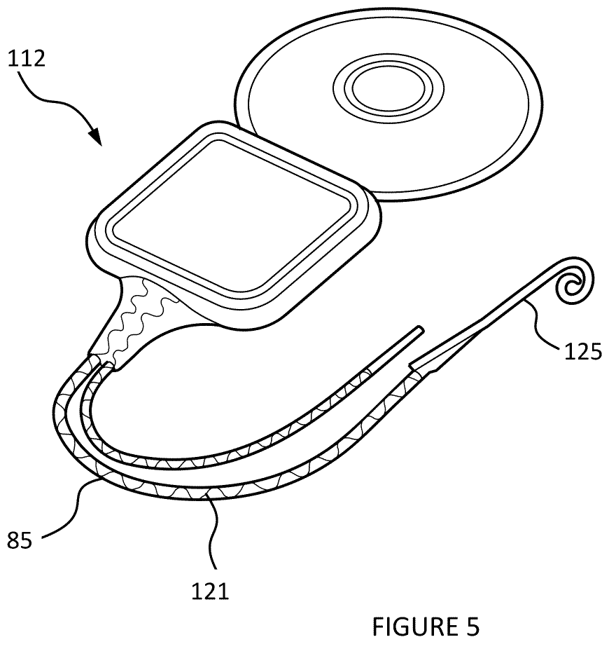

FIG. 5 depicts an isometric view of a cochlear implant 112 corresponding to the cochlear implant 112 detailed above.

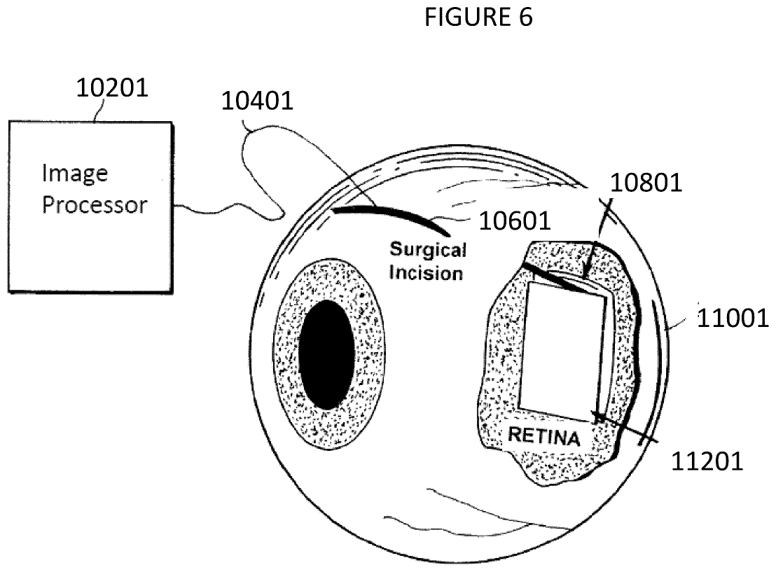

FIG. 6 presents an exemplary embodiment of a neural prosthesis in general, and a retinal prosthesis and an environment of use thereof, in particular, the components of which can be used in whole or in part, with some of the teachings herein. In some embodiments of a retinal prosthesis, a retinal prosthesis sensor-stimulator 10801 is positioned proximate the retina 11001. In an exemplary embodiment, photons entering the eye are absorbed by a microelectronic array of the sensor-stimulator 10801 that is hybridized to a glass piece 11201 containing, for example, an embedded array of microwires. The glass can have a curved surface that conforms to the inner radius of the retina. The sensor-stimulator 10801 can include a microelectronic imaging device that can be made of thin silicone containing integrated circuitry that convert the incident photons to an electronic charge.

An image processor 10201 is in signal communication with the sensor-stimulator 10801 via cable 10401 which extends through surgical incision 00601 through the eye wall (although in other embodiments, the image processor 10201 is in wireless communication with the sensor-stimulator 10801). The image processor 10201 processes the input into the sensor-stimulator 10801 and provides control signals back to the sensor-stimulator 10801 so the device can provide processed output to the optic nerve. That said, in an alternate embodiment, the processing is executed by a component proximate with or integrated with the sensor-stimulator 10801. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 10801 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

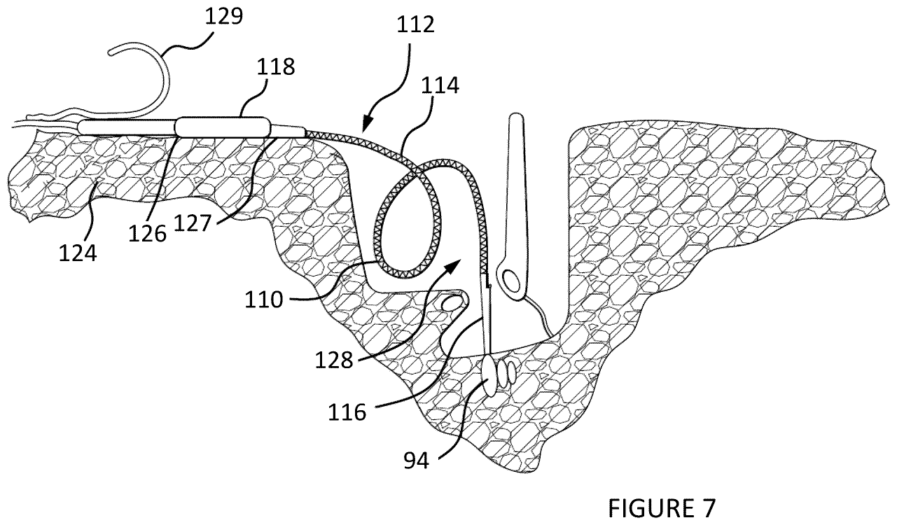

FIG. 7 depicts an exemplary cochlear implant 112 implanted in a recipient just before “closing” during the surgical implantation process. As can be seen, receiver/stimulator 118 lies in a bed 126 formed in the mastoid bone 124 of a recipient, with a skin flap 129 of the scalp of the recipient's head folded back. The bed 126 provides a space for location of the receiver/stimulator 118 to retain the receiver/stimulator in place in the patient's skull and to minimize protrusion of the receiver/stimulator package from the skull when in place after the skin flap 129 is placed back over the receiver/stimulator. A channel 127 is also provided to accommodate the base of the stimulating assembly 85 and the portion of the helix region 114 (or the transition region, if such is that long) of the lead assembly located therein that extends from the receiver/stimulator 118. A hole is drilled into the mastoid bone to allow the electrode array assembly 125 to enter the cavity established by the middle ear and the mastoid cavity 128 and provide access to the round window of the cochlea 94. The area of bone that is removed to provide access to the cochlea 94 is referred to as the mastoid cavity 128. FIG. 7 shows that the stimulating assembly 85 is configured to be long enough to permit the surgeon to manipulate the stimulating assembly 85 into the cochlea 94, as well as to take account for any growth in the patient's skull, if implanted at a young age. Accordingly, in some exemplary embodiments, the surgeon sometimes forms a loop in the transition region of the electrode array assembly that is then placed in the mastoid cavity 128, to account for any excess lead length. However, as will be detailed below, in some alternate exemplary embodiments, this loop is not formed, and alternate actions/arrangements are provided to account for the extra length of the lead.

It is noted that in at least some exemplary embodiments, a portion of the helix region and/or the transition region of the stimulating assembly 85 can be tucked underneath a bony overhang of bone forming a portion of the boundary of a mastoid cavity. Such will be described below in greater detail. In an exemplary embodiment, this can have utilitarian value in that such can secure, or otherwise at least temporarily retain, a portion of the stimulating assembly 85 at a given location. In this regard, the retained portion is separated from the skin overhanging the mastoid cavity 128 by the bony overhang. Still further, in an exemplary embodiment, at least during the surgical procedure, while the portion that is retained underneath the bony overhang is so retained, it is easier to close the skin flap 129. This is because, by way of example only and not by way of limitation, the stimulating assembly 85 is retained from “springing up” out of the mastoid cavity 128. In this regard, it is noted that in at least some exemplary embodiments, the makeup of the stimulating assembly 85, at least with respect to the portions of the helix region and/or the transition region, or at least the portion extending between the receiver/stimulator 118 and the electrode array 125 (the lead assembly 110), or at least a portion thereof, is clastic in nature, in that it has a desire to “spring back” or otherwise return to a first orientation when placed in a second orientation (e.g., returning to a generally straight orientation). In this regard, in an exemplary embodiment, the lead assembly behaves in a manner somewhat analogous to a rubber band, where once the rubber band is unrestrained, it returns to a given shape. In this regard, in an exemplary embodiment, it is due to the clastic tendencies of the lead assembly, or at least the clastic tendencies of some of the material that makes up the lead assembly, that result in the phenomenon of the lead assembly springing out of the mastoid cavity 128. In this regard, in at least some instances, it can be relatively difficult to place the lead assembly 110 in an orientation that will result in the lead assembly 110 remaining within the mastoid cavity 128 at least enough so that the closing process can be executed. Because of this, it can be sometimes difficult to maintain the lead assembly 110 underneath and/or in contact with the bony overhang. Still further, even after the skin flap 129 is secured back in place, after closing, the lead assembly can “migrate” away from the aforementioned bony overhang and come into contact with the skin (the underside of the skin), and can rub/irritate the bottom of the skin in that the stimulating assembly can put pressure on the underside of the skin if it comes into contact with the underside of the skin in general, and if the elasticity of the stimulating assembly is such that it puts an upward force on to the underside of the skin. In some scenarios, the stimulating assembly can rub through the skin to extrude out of the skin. This tends to be undesirable with at least some recipients. In an exemplary embodiment, this can be due to the elastic tendencies of the lead assembly 110, or at least a portion thereof. Accordingly, in an exemplary embodiment, there is an implanted cochlear implant that includes a stimulating assembly having a portion thereof extending from the exit of the channel to the cochlea that is not in contact with skin of the recipient, where the portion extending between the channel exit and the cochlea is about 30 mm, 35 mm, 40 mm, 45 mm, 50 mm, 55 mm, 60 mm or more in length or any value or range of values therebetween in about 1 mm increments. An exemplary embodiment that, in some embodiments, thereof, has utilitarian value in that some and/or all of the aforementioned difficulties/phenomena can be alleviated or otherwise prevented, will now be described.

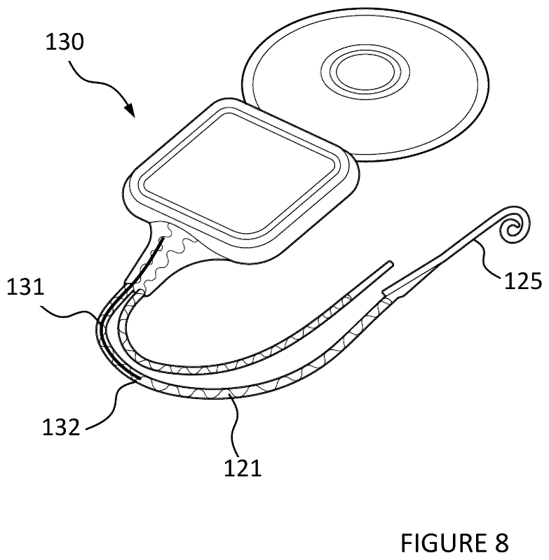

FIG. 8 depicts an exemplary cochlear implant 130 having the functionality of cochlear implant 112 detailed above. Indeed, in an exemplary embodiment, cochlear implant 130 is identical to cochlear implant 112 and/or any other cochlear implant having a stimulating assembly 85, except that in this exemplary embodiment, cochlear implant 130 includes a device configured to prevent, or at least resist, movement of at least a portion of the lead assembly 110 of the stimulating assembly 85, at least in a manner greater than that with respect to conventional cochlear implants. More specifically, in an exemplary embodiment, FIG. 8 includes a stimulating assembly 132 of an implantable stimulating device, such as by way of example only and not by way of limitation, a cochlear implant, that includes a lead assembly made at least partially of material having elasticity/elastic qualities. That is, in the absence of at least some of the teachings detailed herein, the lead assembly would have a tendency or otherwise a “desire,” due to the material properties associated with the lead assembly (e.g., the use of silicone to establish a body thereof, the use of spring-like electrical lead wires, etc.), to return to a first orientation when placed into a second orientation (e.g., spring away from the bony overhang noted above, uncoil, wind upwards towards the underside of the skin from a position where the array was away from the skin, etc.). In an exemplary embodiment, as noted above, in the absence of at least some of the teachings detailed herein, this can result in the lead assembly extending out of the mastoid cavity during the surgical procedure implanting the cochlear implant 130. Accordingly, in this exemplary embodiment, this device is configured to resist movement of at least a portion of the lead assembly, wherein the movement of the lead assembly is due to the elasticity of the lead assembly. In an exemplary embodiment, the movement associated with the elasticity is resisted or otherwise prevented from occurring due to a structure co-located with the lead assembly. In an exemplary embodiment, this entails a malleable portion 131, as can be seen in FIG. 8.

Briefly, it is noted that all disclosures herein regarding to resistance of movement also corresponds to a disclosure of the prevention of movement, and vice versa. Still further, all disclosure herein with regard to resistance of movement and/or prevention of movement corresponds to a disclosure of maintenance of a given orientation and/or position of the electrode array, and vice versa. Still further, all disclosure herein with regard to these aforementioned features also corresponds to a disclosure of enabling the positioning of the electrode array at a given location and subsequently maintaining that positioning. In this regard, the malleable feature can be considered to provide a dual role of both resisting movement, while also enabling the relatively precise positioning of the lead assembly.

It is noted that FIG. 8 depicts a quasi-conceptual schematic of a cochlear implant 112 including the aforementioned structure. In this regard, it is noted that the structure 131 is depicted as being located within the stimulating assembly 132. As will be described below, in some alternative embodiments, the aforementioned structures will be located outside the stimulating assembly 132. To be clear, some additional exemplary embodiments will be described below. The focus of this portion of the specification is to describe the general concepts of at least some exemplary embodiments with respect to an exemplary embodiment where the structure 131 is embedded in a body establishing the lead assembly 110, where, as will be detailed below, other exemplary embodiments exist where the structure is co-located with the lead assembly 110, but the structure is located outside the body establishing the lead assembly 110.

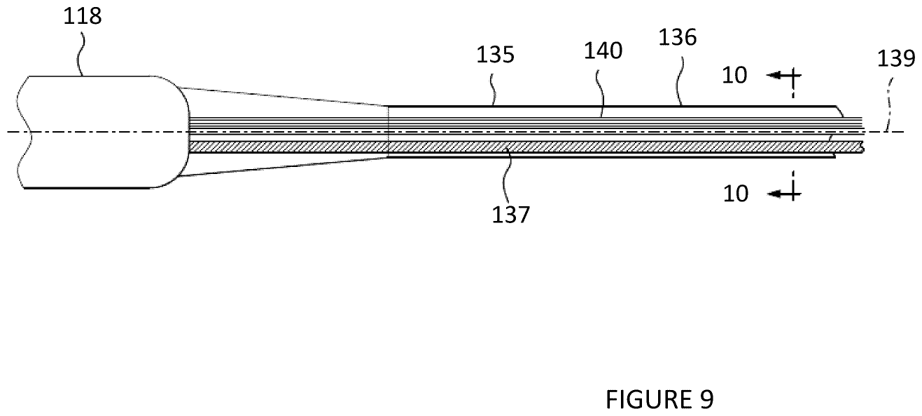

FIG. 9 depicts an exemplary cross-sectional view of an exemplary embodiment of a cochlear implant having a receiver/stimulator 118 from which extends a stimulating assembly 135 that includes a lead assembly corresponding to any of the lead assemblies detailed above, that also includes a malleable metal wire 137, corresponding to the structure 131 of FIG. 8, embedded in the body 136 establishing the lead assembly. As can be seen, wire leads 140 extend from the receiver/stimulator 118. In an exemplary embodiment, these wire leads 140 are embedded in silicone, which establishes the body 136 of the lead assembly. The wire leads depicted in FIG. 9 are depicted as leads that are straight. However, as will be detailed below, leads in a helix arrangement can be utilized in some alternate embodiments (straight leads and leads in a helix configuration can be combined in some embodiments). Additional details of this will be described below.

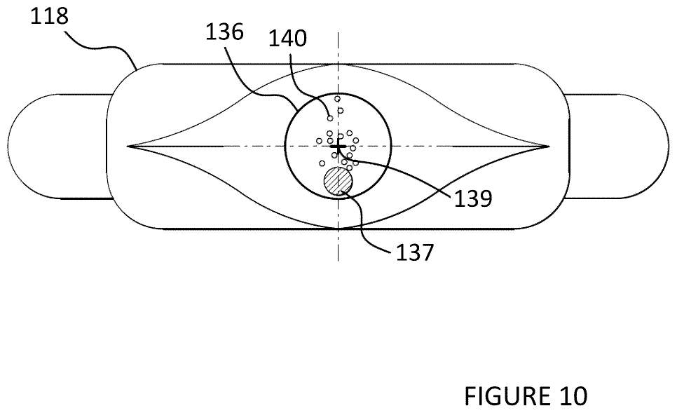

FIG. 10 depicts a cross-sectional view of the stimulating assembly 135, along with the receiver/stimulator 118 in the background, where the bottom of the receiver/stimulator 118 corresponds to the surface that is placed against the mastoid bone when placed in the bed 126 (i.e., the bottom of the bed contacts the bottom of the receiver/stimulator). As can be seen, malleable wire 137 is embedded in the silicone body 136 of the stimulating assembly 135, and is orientated such that it is closer to the bottom of the receiver/stimulator than the top of the receiver/stimulator.

In an exemplary embodiment, the metal wire 137 is made of platinum or some other “soft” metal. That said, in some embodiments, depending on the dimensions, a stainless steel or the like could be used (providing that the diameter was thin enough to enable the bending having utilitarian value detailed herein). Other metals and alloys can be utilized. By way of example only and not by way of limitation, Platinum-Tungsten alloys can be used. Alternatively, and/or in addition to this Prascodymium-Ruthenium can be used. MP35N can be used. Any metal and/or alloy that is malleable in a given structural configuration that can enable the teachings detailed herein and/or variations thereof to be practiced can be utilized in at least some embodiments. Other types of material can be utilized as well, such as by way of example only and not by way of limitation, a plastically deformable polymer, again providing that the teachings detailed herein and/or variations thereof can be practiced.

In an exemplary embodiment, the diameter of a cross-section of the malleable structure, which cross-section can have a circular cross-section, lying on a plane normal to the longitudinal axis thereof, is about 0.2 mm, although greater or smaller diameters can be utilized. In an exemplary embodiment, the diameter, which can be a maximum diameter, is about 0.1 mm, 0.15 mm, 0.2 mm, 0.25 mm or about 0.3 mm or any value or range of values there between in about 0.01 mm increments.

In an exemplary embodiment, the malleable structures detailed herein are not utilized to conduct electricity or signals. Instead, in some embodiments, they are only used for and configured to be used for the spatial maintenance features described herein. In an exemplary embodiment, the malleable structures are structurally different (significantly structurally different in most embodiments) than the electrical lead wires extending from the receiver stimulator of the implant to the electrode array. Indeed, in an exemplary embodiment, the diameter of the malleable structure(s) is an order of magnitude larger than that of a given lead wire. Moreover, as detailed herein, the malleable structures impart spatial maintenance capabilities of the elongate body that are not achieved in the presence of the lead wires alone. In is regard, in an exemplary embodiment, the malleable structures are configured to enable positioning of the elongate stimulating assembly 85 at locations, with the receiver stimulator and the electrode array held in place, and the elongate stimulating assembly unrestrained, at locations that cannot be positioned without the malleable structure (i.e., if the malleable structure was not present, but just the leads were present), all other things being equal. In an exemplary embodiment, the elongate simulating assembly can be placed into a configuration where the elongate simulating assembly subtends an angle of at least 90 degrees, 120 degrees, 150 degrees. 175 degrees, 180 degrees, 195 degrees, 210 degrees, 230 degrees, 250 degrees, 275 degrees, 300 degrees, 330 degrees and/or 360 degrees or more, and maintain that configuration without any component of the implant being secured to anything (e.g., the implant simply laying on a table, etc.).

As seen in FIG. 9, the lead assembly of the stimulating assembly 135 has a longitudinal axis 139. As can be seen, the wire 137 is located further away from the longitudinal axis 139 than the wire leads 140, or at least one wire lead. In this regard, as can be seen, the malleable wire 137 is located further away from the longitudinal axis than at least one of the electrical leads 140. While in some embodiments, the malleable wire 137 is located further from the longitudinal axis than any of the wire leads, in some alternate embodiments, some wire leads are located further from the longitudinal axis or located the same distance from the longitudinal axis as the wire 137, while one or more other wire leads are located closer to the longitudinal axis 139 than the malleable wire 137, or at least located, with respect to the closest approach of the wire leads, at the same distance from the longitudinal axis as the malleable wire (at its closes approach). Thus, in an exemplary embodiment, the extra-cochlear portion of the stimulating assembly 85 includes a plurality of electrical lead wires in electrical communication with the array of electrodes and a malleable component extending in an elongate manner such that the malleable component is located further away from a longitudinal axis of the extra-cochlear portion than at least one of the electrical leads of the plurality of electrical leads.

It is further noted that in an exemplary embodiment, at least a portion of the malleable component is located further away from or the same distance from a longitudinal axis of the extra-cochlear portion than a portion of least one of the electrical leads of the plurality of electrical leads.

Still further, in an exemplary embodiment, the malleable wire 137 is located, with respect to the longitudinal axis, at a location where its greatest distance (e.g., the surface facing away from the longitudinal axis) is located no closer than the closest distance (e.g., the surface facing towards the longitudinal axis) of at least one wire lead. That is, if the malleable wire were to orbit about the longitudinal axis, and the at least one wire lead were to remain stationary, the orbit of the malleable wire would cause the malleable wire to strike the at least one malleable lead. That said, as noted above, in an alternative embodiment, if the malleable wire were to orbit about the longitudinal axis, and the at least one wire lead were to remain stationary, the orbit of the malleable wire would not strike the at least one lead, but instead will go around the at least one lead.

As noted above, the embodiment of FIGS. 9 and 10 is depicted as having straight electrode wires embedded in the silicone body. With respect to the cross-section depicted in FIG. 10, only some of the electrode wires 140 are depicted for purposes of clarity. It will be noted that in at least some exemplary embodiments, there are 22 electrical lead wires, one electrical lead wire for each electrode 92. That said, in some alternate embodiments, more or fewer electrode wires are present.

Thus, in view of the above, in an exemplary embodiment, there is an elongate stimulation assembly of a stimulating implant, such as a cochlear implant, comprising an intra-cochlear portion including an array of electrodes (e.g., region 117 of FIG. 2), and an extra-cochlear portion extending from the intra-cochlear portion (e.g., lead assembly 110 along with the proximal region 116, again with reference to FIG. 2). In an exemplary embodiment, the extra-cochlear portion includes a plurality of electrical lead wires in electrical communication with the array of electrodes and a malleable component (e.g., malleable wire 137) extending in an elongate manner such that the malleable component is located further away from a longitudinal axis 139 of the extra-cochlear portion than at least one of the electrical leads 140 of the plurality of electrical leads 140. In at least some exemplary embodiments, the malleable component is a metallic element which, as detailed above, in an exemplary embodiment, is a metal wire embedded in the lead portion of the stimulating assembly 85. In an exemplary embodiment, the malleable wire 137 is embedded in silicone, which silicone forms the silicone body in which at least some of the wire leads are also embedded.

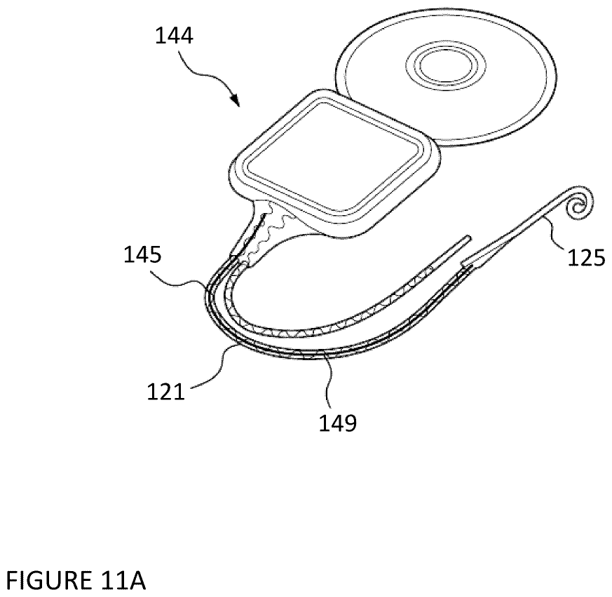

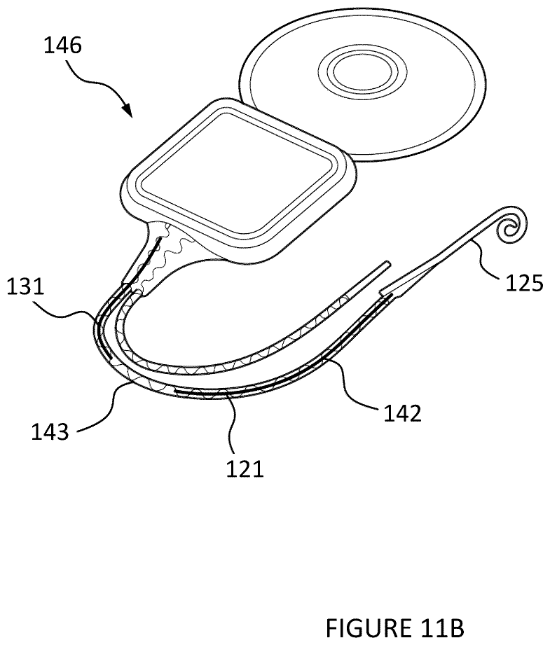

FIG. 8 depicts structure 131 extending only partially along the length of the stimulating assembly 132. In this regard, in an exemplary embodiment, the extra-cochlear region of the stimulating assembly 132 is between about 70 and 80 mm (e.g., about 70, 71, 72 73, 74, 75, 76, 77, 78, 79, or about 80 mm), and structure 131 extends about 25 mm, and thus the malleable region of the stimulating assembly extends about 25 mm. It is noted that the lengths of extension of the structure 131 can be greater or smaller in some embodiments. In an exemplary embodiment, it can extend about 5 mm, 10 mm, 15 mm, 20 mm, 25 mm, 30 mm, 35 mm, 40 mm, 45 mm, 50 mm, 55 mm, 60 mm, 65 mm, or about 70 mm or more or any value or range of values therebetween in about 1 mm increments (e.g., 32 mm, 54 mm, about 7 mm to about 52 mm, etc.). Indeed, as can be seen in FIG. 11A, an exemplary alternate cochlear implant 144 includes a stimulating assembly 145 that includes structure 149 that extends at least substantially the full length between the receiver/stimulator 118 and the electrode array assembly 125. Further, in at least some exemplary embodiments, the structure 149 can further extend into the proximal region 116 of the electrode array assembly 125.

It is further noted that in an exemplary embodiment, the structure 131 (or any of the related structures detailed herein and/or variations thereof) can also be or in the alternative be implemented in the elongate assembly that supports the extra-cochlear electrode (e.g., the electrode that provides the “return” (at least in part) for the current flowing from the electrodes located in the cochlea (the other of the elongate structures of FIG. 8). Indeed, in an exemplary embodiment, the teachings detailed herein regarding the structure 131 and/or alternate embodiments thereof are applicable to any elongate structure that has the aforementioned elastic tendencies associated with stimulating assembly 132 (or even structures that do not have such elastic tendencies).

As depicted in FIG. 9, the structure forming the malleable wire 137 extends from the housing of the receiver/stimulator 118. In an exemplary embodiment, the structure can be connected to the housing of the receiver/stimulator 118. Indeed, in an exemplary embodiment, the end of the malleable wire 137 can be rigidly fixed to the housing. That said, in an alternative embodiment, the malleable wire 137 can be offset from the housing, as will be seen in an alternate exemplary embodiment below. In this regard, because, in at least some exemplary embodiments, malleable portion has utility at locations away from the housing of the receiver/stimulator 118, and, in at least some embodiments, there is little to no utilitarian value with respect to locating the malleable portion at and/or proximate the housing, the malleable portion can be offset from the housing of the receiver/stimulator 118. Any location of the malleable structures that can enable the teachings detailed herein to be practiced can be utilized in at least some embodiments.

FIG. 11B depicts an alternate cochlear implant 146 that includes a stimulating assembly 143 that includes structure 131 as detailed above with respect to FIG. 8 and structure 142 that extends from the electrode array 125 (although in other embodiments, it can be offset from the electrode array 125) towards the receiver/stimulator about 25 mm, leaving a section between structure 131 and structure 142 that is not malleable (a structure that is about 25 mm, in an exemplary embodiment). Thus, with respect to this embodiment, there is a receiver/stimulator of a cochlear implant connected to a lead assembly at a first location of the lead assembly. The cochlear implant, includes a first structural component (structure 131, which can be a malleable wire corresponding to malleable wire 137 detailed above, or can correspond to any other of the structures detailed herein or other structures that can enable the teachings detailed herein to be practiced) separate) and second structural component (structure 142, which can be a malleable wire corresponding to malleable wire 137 detailed above, or can correspond to any other of the structures detailed herein or other structures that can enable the teachings detailed herein to be practiced) separate from the first structural component. In this exemplary embodiment, the first structural component is located proximate the receiver/stimulator, and the second structural component is located remote from the first structural component.

Still further, in an exemplary embodiment, the first structural component is configured to prevent, or at least resist, movement of at least a first portion of the lead apparatus of the stimulating assembly of the cochlear implant, which movement can correspond to the movement resulting from the elasticity of at least a portion of the material making up the lead assembly (e.g., silicone), the first portion of the lead apparatus being proximate the first structural component. Also, the second structural component is configured to prevent, or at least resist, movement of at least a second portion of the lead apparatus, again, which movement can correspond to the movement resulting from the elasticity of at least a portion of the material making up the lead assembly, the second portion of the lead apparatus being proximate the second structural component.

Still further in view of FIG. 11B, the lead assembly of the stimulating assembly of cochlear implant 146 includes a third portion unrestrained from movement due to elasticity of the third portion, the third portion being located between the first portion and the second portion (e.g., between structure 131 and 142 of FIG. 11B.). In some exemplary embodiments, there is an electrode array located at a location, with respect to the lead assembly, opposite to the location where the lead assembly connects to the receiver/stimulator 118.

In an exemplary embodiment, the malleable portions of the cochlear implants detailed herein can have utilitarian value in that it can enable the lead assembly, or at least a portion thereof, to be deformed to an orientation that is deemed utilitarian with respect to the anatomy of a recipient (albeit in a potentially altered state due to the surgery (e.g., the creation of the mastoid cavity) which orientation will be maintained after the establishment of the orientation. In this regard, in an exemplary embodiment, the cochlear implant 130 is configured to resist the movement of the at least a portion of the lead assembly due to the elasticity via a structure, such as malleable wire 137, co-located with the lead assembly, wherein the structure is configured to deform upon the application of sufficient force. This applied force is greater than a force applied to the structure via the elasticity (and, in some embodiments, opposite that force). As will now be described, this force moves the lead assembly along with the deformation so that the lead assembly can be positioned, or at least a portion of the lead assembly can be positioned, at a desired orientation, while the malleability of the structure holds the lead assembly/portion thereof at the desired position/orientation after the positioning.

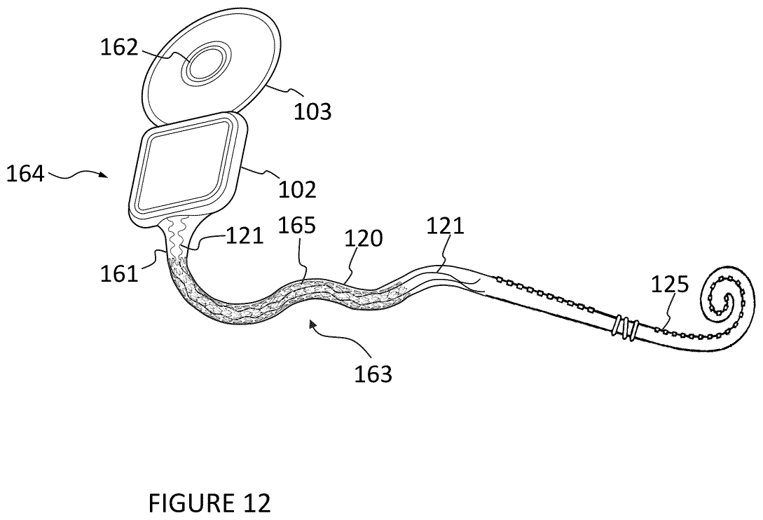

FIG. 12 depicts an exemplary cochlear implant 164 having the functionality of cochlear implant 112 detailed above and features associated with cochlear implant 130 discussed above, but in a different manner. Just as is the case with implant 130, in an exemplary embodiment, cochlear implant 164 is identical to cochlear implant 112 of FIG. 1 and/or any other cochlear implant having an elongate stimulating assembly, in an exemplary embodiment, and in other embodiment, can be different in one or more ways (all embodiments are exemplary unless otherwise noted), and just as is the case with implant 130, in this exemplary embodiment, cochlear implant 164 includes a device configured to prevent, or at least resist, movement of at least a portion of the lead assembly of the stimulating assembly 163, at least in a manner greater than that with respect to conventional cochlear implants. More specifically, in an exemplary embodiment, FIG. 12 includes a stimulating assembly 163 of an implantable stimulating device, such as by way of example only and not by way of limitation, a cochlear implant, that includes a lead assembly made at least partially of material having elasticity/elastic qualities. Accordingly, in this exemplary embodiment, as with the embodiment of FIG. 8, this device is configured to resist movement of at least a portion of the lead assembly, wherein the movement of the lead assembly is due to the elasticity of the lead assembly. In an exemplary embodiment, the movement associated with the elasticity is resisted or otherwise prevented from occurring due to a structure co-located with the lead assembly. In an exemplary embodiment, this entails a cage 165, as can be seen in FIG. 12.

It is noted that FIG. 12 depicts a quasi-conceptual schematic of a cochlear implant 164 including the aforementioned structure. In this regard, it is noted that the structure 165 (cage structure) is depicted as being located within the stimulating assembly 163. As will be described below, in some alternative embodiments, the aforementioned cage structure 165 will be located outside the stimulating assembly 163. In this embodiment, by way of example, the cage 165 is embedded in a body 120 establishing the lead assembly 110 of the implant, where, as will be detailed below, other exemplary embodiments exist where the cage is co-located with the lead assembly 110, but the cage is located outside the body 120 establishing the lead assembly 110. In an embodiment, the body 120 is established by a silicone molding. Also shown in FIG. 12 is a body of silicone 161 that establishes an end of the elongate stimulating assembly, and provides for a transition of such to the receiver stimulator 118 (hereinafter, element 161 is often referred to as transition body 161).

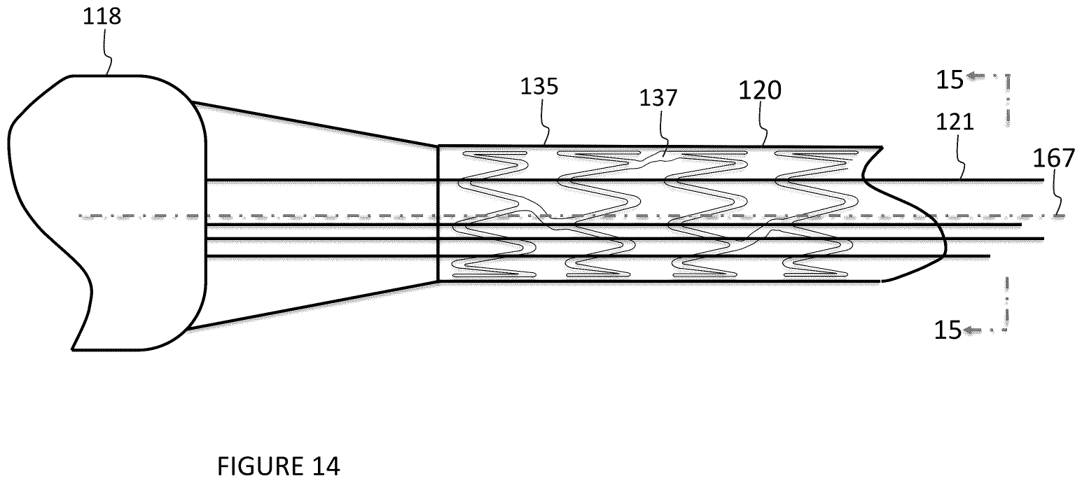

FIG. 14 depicts an exemplary cross-sectional view of an exemplary embodiment of a cochlear implant having a receiver/stimulator 118 from which extends a stimulating assembly 163 that includes a lead assembly corresponding to any of the lead assemblies detailed above, that also includes a cage 165, corresponding to the cage of FIG. 12, embedded in the body 120 establishing the lead assembly. As can be seen, wire leads 121 extend from the receiver/stimulator 118. In an exemplary embodiment, these wire leads 121 are embedded in silicone, which establishes the body 120 of the lead assembly. The wire leads depicted in FIG. 14 are depicted as leads that are straight. However, as will be detailed below, leads in a helix arrangement and in a wavy and in a serpentine and in a zig-zag arrangement can be utilized in some alternate embodiments (straight leads and leads in a helix configuration and leads in a zig-zag and in a wavy and in a serpentine configuration can be combined in some embodiments (one or more with one or more others). Additional details of this will be described below.

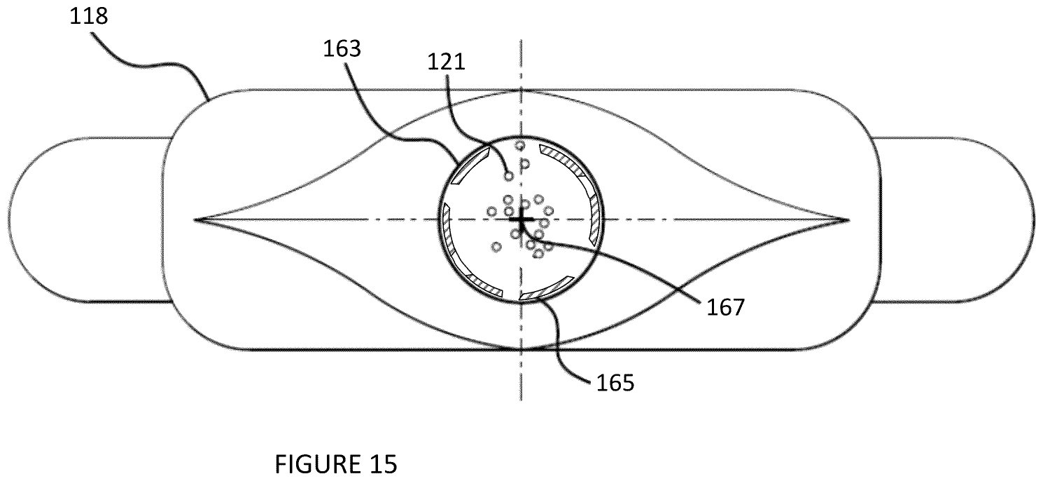

FIG. 15 depicts a cross-sectional view of the stimulating assembly 163, along with the receiver/stimulator 118 in the background, where the bottom of the receiver/stimulator 118 corresponds to the surface that is placed against the mastoid bone when placed in the bed 126 (i.e., the bottom of the bed contacts the bottom of the receiver/stimulator). As can be seen, cage 165 is embedded in the silicone body (in an embodiment, everything in the cross-section of FIG. 15 not the lead wires and the cage is silicone, including the areas between the cage portions) of the stimulating assembly 163, and is orientated such that it is closer to the bottom of the receiver/stimulator than the top of the receiver/stimulator.

As seen in FIG. 14, the lead assembly of the stimulating assembly 163 has a longitudinal axis 167. As can be seen, the cage 165 encompasses the wire leads 121. In an exemplary embodiment, the extra-cochlear portion of the elongate stimulating assembly includes a plurality of electrical lead wires in electrical communication with the array of electrodes and a cage extending in an elongate manner about those lead wires.

It is further noted that in an exemplary embodiment, at least a portion of the cage is located further away from or the same distance from a longitudinal axis of the extra-cochlear portion than a portion of least one of the electrical leads of the plurality of electrical leads.

Still further, in an exemplary embodiment, the longitudinal axis of the cage 165 is offset from the longitudinal axis 167 of the elongate stimulating assembly 163, while in other embodiments, it is concentric with the axis 167.

As noted above, the embodiment of FIGS. 14 and 15 are depicted as having straight electrode wires embedded in the silicone body. With respect to the cross-section depicted in FIG. 15 only some of the electrode wires 121 are depicted for purposes of clarity. It will be noted that in at least some exemplary embodiments, there are 22 electrical lead wires, one electrical lead wire for each electrode 92. That said, in some alternate embodiments, more or fewer electrode wires are present.

Thus, in view of the above, in an exemplary embodiment, there is an elongate stimulation assembly of a stimulating implant, such as a cochlear implant, comprising an intra-cochlear portion including an array of electrodes (e.g., region 117 of FIG. 2), and an extra-cochlear portion extending from the intra-cochlear portion (e.g., lead assembly 110 along with the proximal region 116, again with reference to FIG. 2). In an exemplary embodiment, the extra-cochlear portion includes a plurality of electrical lead wires in electrical communication with the array of electrodes and a cage 165 extending in an elongate manner such that cage encompasses the wire leads of the extra-cochlear portion. In at least some exemplary embodiments, the cage is a metallic element which, can be, in an exemplary embodiment, a metal wire mesh or grid structure embedded in silicone of the stimulating assembly 120. In an exemplary embodiment, the cage 165 is embedded in silicone, which silicone forms the silicone body in which at least some of the wire leads are also embedded.

FIG. 12 depicts cage 165 extending only partially along the length of the stimulating assembly 163. In this regard, in an exemplary embodiment, the extra-cochlear region of the stimulating assembly 163 is between about 30 and 200 mm (e.g., about 50, 61, 72 93, 154, 165, 170, 180, 190 or 200 mm or any value or range of values between (inclusive) 30 mm and 200 mm in 0.1 mm increments), and cage 165 extends about 60 mm (where the total length of the assembly is 75 mm) in this exemplary embodiment, and thus the region of the stimulating assembly extends about 60 mm. It is noted that the lengths of extension of the cage 165 can be greater or smaller than these values in some embodiments. In an exemplary embodiment, the cage 165 can extend less than, greater than and/or equal to 15 mm, 20 mm, 25 mm, 30 mm, 35 mm, 40 mm, 45 mm, 50 mm, 55 mm, 60 mm, 65 mm, 70 mm, 80 mm, 90 mm, 100 mm, 110 mm, 120 mm, 130 mm, 140 mm, 150 mm, 160 mm, 170 mm, 180 mm, 190 mm or 200 mm or more or any value or range of values therebetween in 1 mm increments (e.g., 44 mm, 66 mm, about 33 mm to about 155 mm, etc.). Indeed, in an exemplary alternate cochlear implant 164, there is a stimulating assembly 163 that includes a cage structure that extends at least substantially the full length between the receiver/stimulator 118 and the electrode array assembly 125. Further, in at least some exemplary embodiments, the cage structure can further extend into the proximal region 116 of the electrode array assembly 125.

It is further noted that in an exemplary embodiment, the cage 165 (or any of the functionally analogous structures detailed herein and/or variations thereof) can also be or in the alternative be implemented in the elongate assembly that supports the extra-cochlear electrode (e.g., the electrode that provides the “return” (at least in part) for the current flowing from the electrodes located in the cochlea). Indeed, in an exemplary embodiment, the teachings detailed herein regarding the cage 165 and/or alternate embodiments thereof are applicable to any elongate structure that has the aforementioned elastic tendencies associated with various stimulating assemblies (or even structures that do not have such elastic tendencies). More on this below.

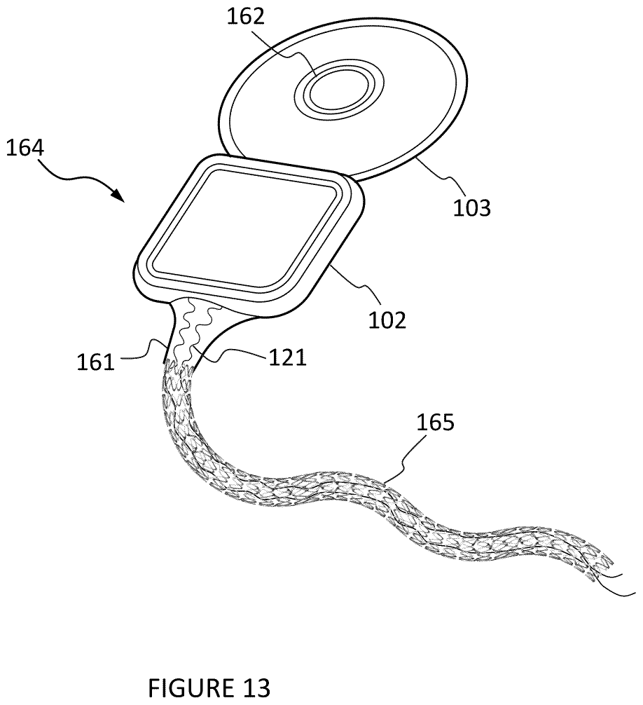

In some embodiments, the structure forming the cage extends from the housing of the receiver/stimulator 118 while in other embodiments, this is not the case. In an exemplary embodiment, the cage 165 can be connected to the housing of the receiver stimulator 118 and in other embodiments, the cage 165 is not connected to the housing of the receiver stimulator. In an exemplary embodiment, the end of the cage 165 can be rigidly fixed to the housing of the receiver-stimulator. That said, in an alternative embodiment, the cage 165 can be offset from the housing of the receiver stimulator, as seen in FIG. 13. Any location of the images that can enable the teachings detailed herein to be practiced can be utilized in at least some embodiments.

FIG. 13 depicts a view of implant 164 but without the electrode array and without the body 120, for purposes of case of illustration. Here, the cage 165 can be more clearly seen, and also seen are lead wires 121 extending from the receiver-stimulator. The wire leads depicted in FIG. 13 are depicted as leads that are wavy and serpentine. However, as will be detailed below, leads in a helix arrangement or a zig-zag or a straight arrangement or any other arrangement that can have utilitarian value can be utilized in some alternate embodiments (straight leads and wavy leads in a helix configuration can be combined in some embodiments). Additional details of this will be described below.

In an embodiment, the cage 165 is embedded in a silicone body of the stimulating assembly 163, and is orientated such that it is concentric with an outer surface of the silicone body, while in other embodiments, a longitudinal axis of the body is offset from a longitudinal axis of the cage.

In an exemplary embodiment, the cage 165 is made of platinum or some other “soft” metal. That said, in some embodiments, depending on the dimensions, a stainless-steel cage or the like could be used (providing that the diameter was thin enough to enable the bending having utilitarian value detailed herein). Other metals and alloys can be utilized. Any metal and/or alloy that can enable the teachings detailed herein in a cage structure, such as that which results in a malleable cage by way of example, that can enable the teachings detailed herein and/or variations thereof to be practiced can be utilized in at least some embodiments. Other types of material can be utilized as well, such as by way of example only and not by way of limitation, a plastically deformable polymer, again providing that the teachings detailed herein and/or variations thereof can be practiced.

To be clear, in an exemplary embodiment, the cage 165 is configured to be deformable in accordance with the teachings detailed above with respect to the malleable component so as to hold the elongate simulating assembly in place in accordance with the teachings above with respect to the malleable component. In this regard, by way of example only and not by way of limitation, any of the functional features associated with the malleable component detailed above are also present with respect to the cage component providing that the art enables such unless otherwise noted, all in the interests of textual economy. It is not clear embodiment, any of the teachings herein with respect to the malleable component detailed above corresponds to an alternate disclosure, again in the interest of textual economy, of such with the cage instead of and/or in addition to the malleable component. In an exemplary embodiment, the cage is a malleable structure.

In an exemplary embodiment, the cage has a circular radial profile (albeit a non-solid cross-section-more on this below), lying on a plane normal to the longitudinal axis thereof, that is less than, greater than and/or equal to 0.5, 0.6, 0.7, 0.8, 0.9, 1, 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 5.25, 5.5, 5.75 or 6 mm or any value or range of values therebetween in 0.01 mm increments.

In an exemplary embodiment, the cage(s) detailed herein are not utilized to conduct electricity or signals. Instead, in some embodiments, the cages are only used for and configured to be used for the spatial maintenance features described herein. In an exemplary embodiment, the cages are structurally different (significantly structurally different in most embodiments) than the electrical lead wires extending from the receiver stimulator of the implant to the electrode array. Indeed, in an exemplary embodiment, the diameter of the cage structures (normal to the longitudinal axis) is an order of magnitude or two or anywhere therebetween larger than that of a given lead wire. Moreover, as detailed herein, the malleable structures impart spatial maintenance capabilities of the elongate body that are not achieved in the presence of the lead wires alone. In is regard, in an exemplary embodiment, the malleable structures are configured to enable positioning of the elongate stimulating assembly 163 (or other assemblies) at locations, with the receiver stimulator and the electrode array held in place, and the elongate stimulating assembly unrestrained, at locations that cannot be positioned without the malleable structure (i.e., if the malleable structure was not present, but just the leads were present), all other things being equal. In an exemplary embodiment, the elongate simulating assembly can be placed into a configuration where the elongate simulating assembly subtends an angle of at least 90 degrees, 120 degrees, 150 degrees. 175 degrees, 180 degrees, 195 degrees, 210 degrees, 230 degrees, 250 degrees, 275 degrees, 300 degrees, 330 degrees and/or 360 degrees or more, and maintain that configuration without any component of the implant being secured to anything (e.g., the implant simply laying on a table, etc.).

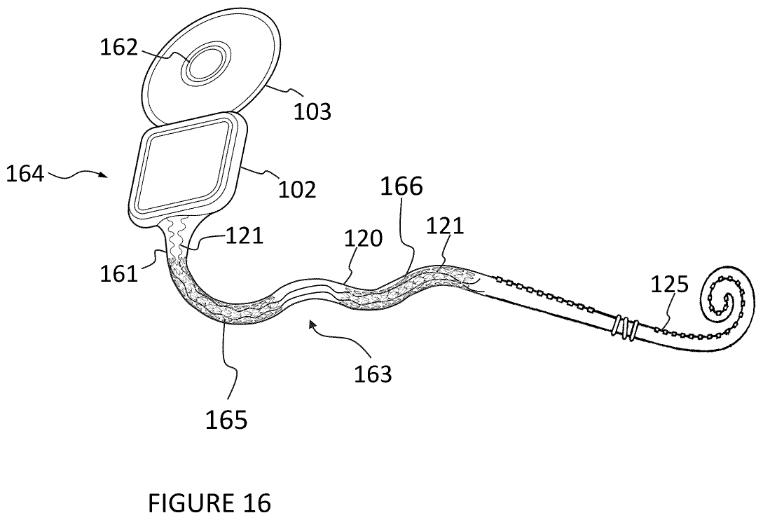

FIG. 16 depicts an alternate cochlear implant 164 that includes a stimulating assembly 163 that includes cage 165 as detailed above with respect to FIG. 12 and cage 166 that extends from a location offset from electrode array 125 (although in other embodiments, cage 166 can extend from electrode array 125) towards the receiver/stimulator about 36 mm, leaving a section between cage 165 and cage 166 that is not controlled by the cage geometry, and, in an embodiment, is not malleable (a structure that is about 15 mm, in an exemplary embodiment). Thus, with respect to this embodiment, there is a receiver/stimulator of a cochlear implant connected to a lead assembly at a first location of the lead assembly. The cochlear implant, includes a first structural component (cage 165, which can be a malleable cage 165 or another type of cage, or can correspond to any other of the structures detailed herein or other structures that can enable the teachings detailed herein to be practiced) and a second structural component (cage 166, which can be a cage corresponding to the basic structural design of cage 165, or different therefrom) detailed above, or can correspond to any other of the structures detailed herein or other structures that can enable the teachings detailed herein to be practiced) separate from the first structural component. In an exemplary embodiment, the first structural component is located proximate the receiver/stimulator, and the second structural component is located remote from the first structural component.

Still further, in an exemplary embodiment, the first cage is configured to prevent, or at least resist, movement of at least a first portion of the lead apparatus of the stimulating assembly of the cochlear implant, which movement can correspond to the movement resulting from the elasticity of at least a portion of the material making up the lead assembly (e.g., silicone), the first portion of the lead apparatus being proximate the first cage. Also, the second cage is configured to prevent, or at least resist, movement of at least a second portion of the lead apparatus, again, which movement can correspond to the movement resulting from the elasticity of at least a portion of the material making up the lead assembly, the second portion of the lead apparatus being proximate the second cage.

Still further in view of FIG. 16 the lead assembly of the stimulating assembly of cochlear implant 164 includes a third portion unrestrained from movement due to elasticity of the third portion, the third portion being located between the first cage and the second cage (e.g., between structure 165 and 166 of FIG. 16.). In some exemplary embodiments, there is an electrode array located at a location, with respect to the lead assembly, opposite to the location where the lead assembly connects to the receiver/stimulator 118.

In an exemplary embodiment, the cages of the cochlear implants detailed herein can have utilitarian value in that it can enable the lead assembly, or at least a portion thereof, to be deformed to an orientation that is deemed utilitarian with respect to the anatomy of a recipient (albeit in a potentially altered state due to the surgery (e.g., the creation of the mastoid cavity)) which orientation will be maintained after the establishment of the orientation. In this regard, in an exemplary embodiment, the cochlear implant of a given embodiment, such as that of FIG. 1, is configured to resist the movement of the at least a portion of the lead assembly due to the elasticity via a structure, such cage 165 and/or cage 166, co-located with the lead assembly, wherein the structure is configured to deform upon the application of sufficient force. This applied force is greater than a force applied to the structure via the elasticity (and, in some embodiments, opposite that force). As will now be described, this force moves the lead assembly along with the deformation so that the lead assembly can be positioned, or at least a portion of the lead assembly can be positioned, at a desired orientation, while, in some embodiments, the malleability of the cages hold the lead assembly/portion thereof at the desired position/orientation after the positioning.

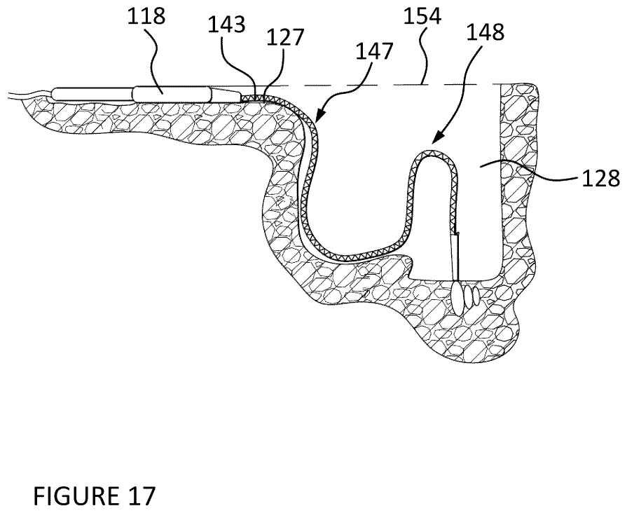

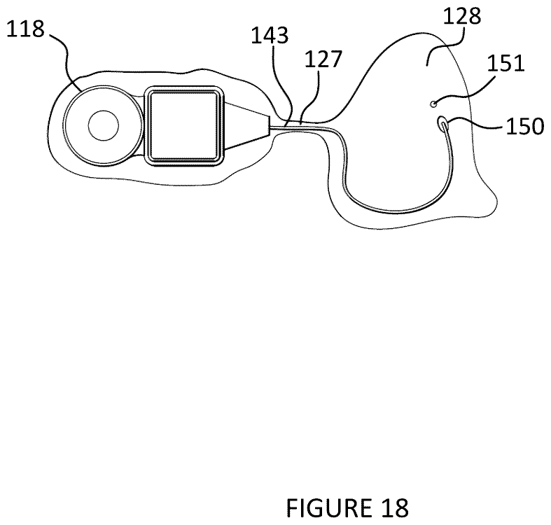

FIG. 17 depicts a side view of an implanted cochlear implant (corresponding to implant 146 noted above by way of example) according to an exemplary embodiment, and FIG. 18 depicts a top view of FIG. 17, showing the mastoid cavity 128 and the middle ear cavity (all skin has been removed for clarity) and the round window 151 and oval window 150 of the cochlea, with the stimulating assembly 143 extending into the oval window 150. Generally, the view of FIG. 17 is corollary to the view of FIG. 7, with some additional details with respect to the surfaces of the mastoid bone that have been removed for implantation of the cochlear implant 164, and to reflect the fact that an exemplary cochlear implant having the malleable structures detailed herein is utilized.

As can be seen, the receiver/stimulator 118 of cochlear implant 144 (which is a proxy for the various cochlear implants, such as 146 and 164 detailed herein, in the interests of textual economy-reference to one corresponds to an alternate embodiment that utilizes one of the others in the interest of textual economy, providing that the art enable such, unless otherwise noted) lies in bed 126 that is cut into the mastoid bone 124. Line 154 represents the “top” of the mastoid bone with respect to the portions thereof that have not been altered for implantation (i.e., it depicts the background rim of the excavations), and is presented in dashed line format for purposes of clarity. A portion of the lead assembly of the stimulating assembly 143 lies in the channel 127. However, the portion of the lead assembly immediately proximate to the channel's exit into the mastoid cavity 128 is bent downward to follow the contour of the surface of the mastoid cavity. In conceptual terms, the lead assembly flows like water over a waterfall (although it can veer to the left or the right, as indicated by FIG. 18, where the direction of flow is from the channel 127 into the mastoid cavity 128). In some embodiments, the bottom surface of the lead assembly remains in contact with the mastoid bone at least proximate to the exit of the channel 127. In some embodiments, the bottom surface of the lead assembly remains at least generally in contact with the mastoid bone at least for a distance of about 1 mm, about 2 mm, about 3 mm, about 4 mm, about 5 mm, or about 6 mm or any value or range of values therebetween in about 0.1 mm increments, with respect to a distance from the exit of the channel 127 into the mastoid cavity 128. In this exemplary embodiment, there is no retention between the channel exit and the surface of the cochlea with respect to the lead assembly (e.g., no fasteners or fixtures holding the lead apparatus in place between those locations, etc.). Still further, in some exemplary embodiments, no part of the mastoid bone or the anatomy forming the middle ear cavity (other than the entrance to the cochlea, if indeed that is deemed part of the middle ear cavity) provides positive resistance to movement of the lead assembly (e.g., due to the overhang), and, in some exemplary embodiments, no part of the mastoid bone or the anatomy forming the middle ear cavity (other than the entrance to the cochlea, if indeed that is deemed part of the middle ear cavity) provides any resistance to movement of the lead assembly (e.g., due to friction forces).

In an exemplary embodiment, the stimulating assembly is configured so as to retain the stimulating assembly within the mastoid cavity entirely due to its own structure without any intervening forces or other resistance from the anatomy of the recipient between the location of the exit of the channel and the entrance of the cochlea (although a portion of the stimulating assembly could still be in contact with the anatomy-it just does not need to be in such contact to achieve the aforementioned functionality). Still further, in an exemplary embodiment, any or all of the aforementioned functionalities can be achieved without looping the stimulating assembly.

An exemplary embodiment includes a stimulating assembly that is configured to achieve any or all of the aforementioned functionalities

Thus, in an exemplary embodiment, the portion of the stimulating assembly extending between the exit of the channel 127 and the entrance of the cochlea (or at least the portion that corresponds to the helix region) is otherwise free to move but for the fact that the malleable structure prevents such movement or otherwise resists such movement, and for the influence of the cochlea and the channel on the stimulating assembly. In an exemplary embodiment, the portion of the stimulating assembly extending between the exit of the channel 127 and the entrance of the cochlea (or at least the portion that corresponds to the helix region) is oriented substantially entirely due to the malleable structure and due to the channel and due to the cochlea (which includes a scenario where there is a portion that does not include the malleable structure-that portion still being oriented due to the malleable structure owing to the fact that the malleable structures establish a trajectory of that portion).

In an exemplary embodiment, the portion of the stimulating assembly extending between the exit of the channel 127 and the entrance of the cochlea (or at least the portion that corresponds to the helix region) is subjected to a restraining force due entirely to the malleable portion and the channel and the cochlea. In an exemplary embodiment, the portion of the stimulating assembly extending between the exit of the channel 127 and the entrance of the cochlea (or at least the portion that corresponds to the helix region) is unrestrained from moving out of the mastoid cavity/upwards towards the inside of the skin by the anatomy of the recipient (save for the influence of the channel and the cochlea).

In some embodiments, the bottom surface of the lead assembly is not in contact with the mastoid bone within the mastoid cavity 128 after exiting the channel 127 within the aforementioned dimensions, but the lead assembly substantially parallels the surface thereof.

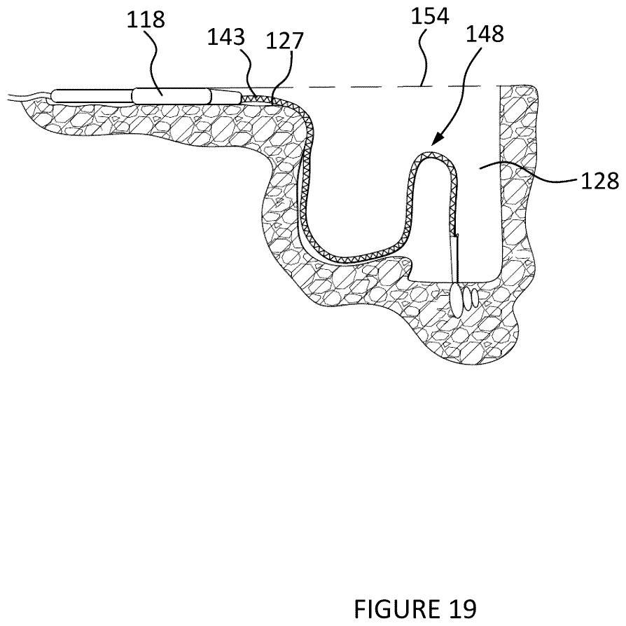

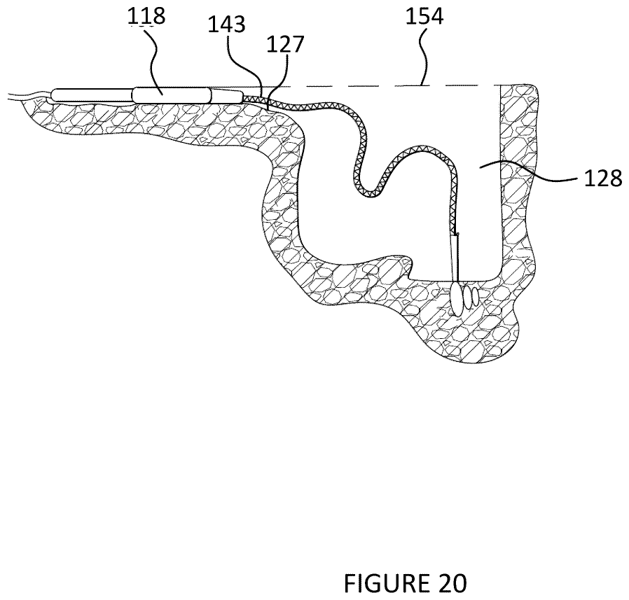

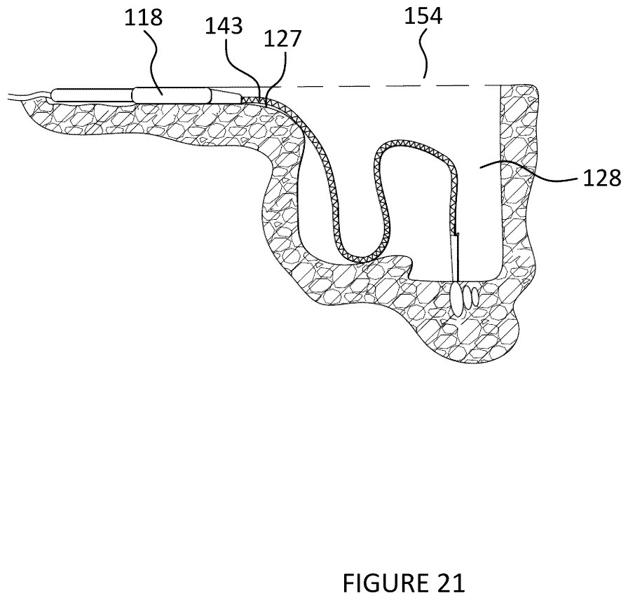

FIGS. 19-22 depict some exemplary arrangements of the stimulating assembly after implantation into the recipient.

The aforementioned bending downward is established at bend 147, which is established by bending or otherwise deforming structure 131 at that location so that it follows the contours of the mastoid bone and extends downward as shown. In an exemplary embodiment, this can be achieved by gripping the stimulating assembly with a pair of tweezers and imparting a twist on to the tweezers thus bending the stimulating assembly such that the malleable structure to forms. The structure 131 establishes the general trajectory of the lead assembly at this relevant area, and thus owing to the properties of the other portions of the lead assembly, the lead assembly generally stays within the mastoid cavity 128, and does not have a tendency to rise above line 154. That said, owing to the fact that this embodiment utilizes cochlear implant 146 (or 146 or 164, etc., again this is presented in the interests of textual economy), which includes structure 142, a second bend in the lead assembly can be located at bend 148, which again establishes a trajectory of the lead assembly at this relevant area, thus further maintaining the lead assembly within the mastoid cavity 128. Again, this can be achieved utilizing a pair of tweezers and subjecting the tweezers to the aforementioned twisting, which bends the stimulating assembly, and thus the forms the malleable structure.

It is noted that the bending of the malleable structures detailed herein can occur anywhere along the length thereof. Any bending of the malleable structures that will resist movement of the lead assembly or otherwise maintain or establish a position of the lead assembly such that it remains below the line 154 of the mastoid cavity can be utilized in at least some exemplary embodiments to practice some embodiments.

Accordingly, an exemplary embodiment includes an implanted cochlear implant having a lead assembly having portions corresponding at least generally to the orientations depicted in the figures herein. However, it is noted that other orientations can be utilized as well. Any orientation of the lead assemblies that is established according to the malleable structures detailed herein and/or other types of structures that can enable the teachings detailed herein can be utilized in at least some exemplary embodiments. Corollary to this is that an exemplary embodiment includes methods of implanting a cochlear implant to have such orientations. In this regard, some exemplary methods will now be described.

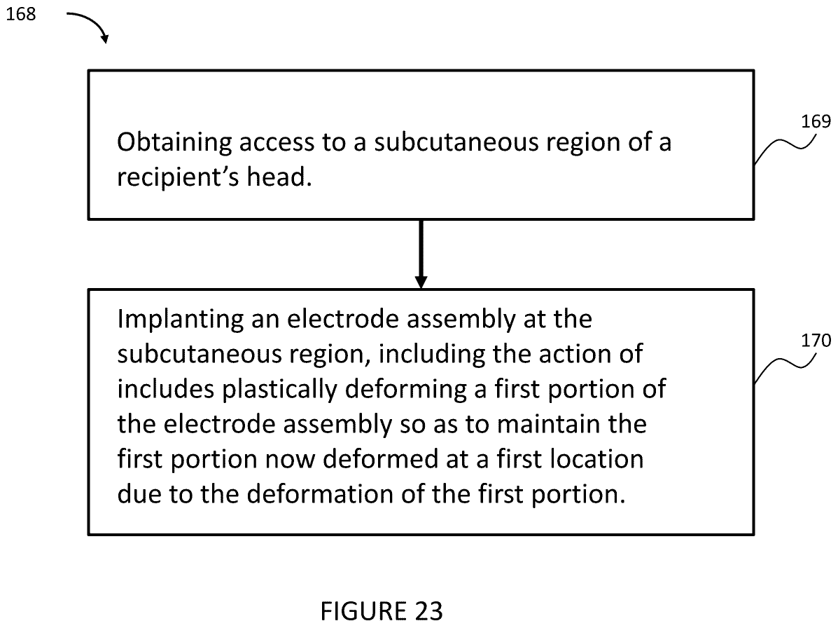

FIG. 23 depicts a flowchart for an exemplary method 168, which includes method actions 169 and 170 (it can include other method actions). Method action 169 entails obtaining access to a subcutaneous region of a recipient's head. In an exemplary embodiment, this can entail cutting into the skin of the recipient to reach the mastoid bone. In an exemplary embodiment, this can further entail excavating the portions of the mastoid bone to establish the mastoid cavity, the bed, and the channel, etc. That said, in an alternate embodiment, method action 169 can be executed by obtaining access to a subcutaneous region, the path to which was previously established by another entity. That is, the person executing method action 169 need not necessarily be the person to cut into the recipient and/or excavate the portions of the mastoid bone, etc. After executing method action 169, method action 170 is executed, which entails implanting a stimulating assembly at the subcutaneous region, wherein the action of implanting the electrode assembly includes plastically deforming a first portion of the stimulating assembly so as to maintain the first portion now deformed at a first orientation due to the deformation of the first portion. In an exemplary embodiment, this entails establishing the bend 147 detailed above, or any of the other bends detailed herein and/or variations thereof. In an exemplary embodiment, this action is established by plastically deforming the malleable components detailed herein, such as for example, the malleable wire. In an exemplary embodiment, this action is established by plastically deforming the cage structures. In an exemplary embodiment, this action is executed by plastically deforming another type of structure that can enable the teachings detailed herein.

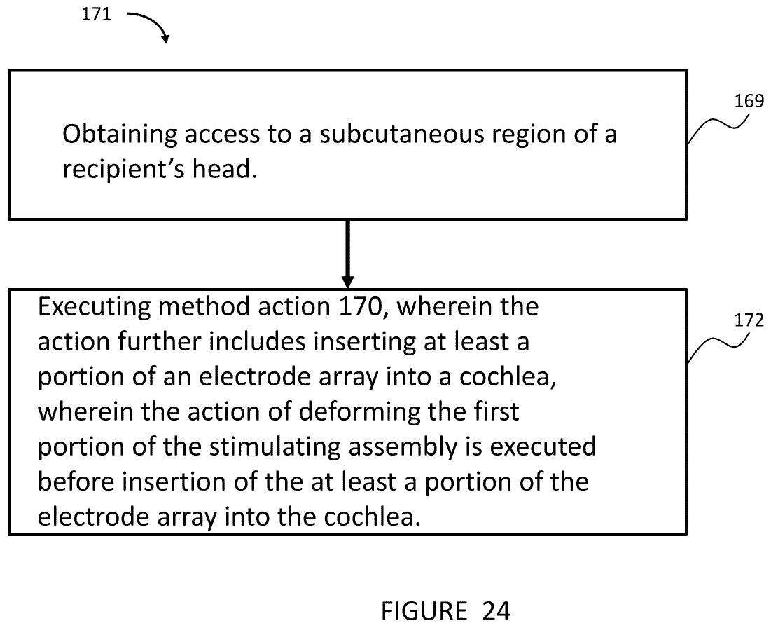

FIG. 24 depicts a variation of method 168, or more accurately, an expansion thereof. Method 171 of FIG. 24 includes method action 169, which is identical to that of method 168. Method 171 further includes method action 172, which entails executing method action 170, wherein the action further includes inserting at least a portion of an electrode array into a cochlea. In method action 172, the action of deforming the first portion of the stimulating assembly is executed before insertion (at least full insertion) of the at least a portion of the electrode array into the cochlea.

That said, in an alternate embodiment, method action 172 entails executing method action 170, wherein the action also further includes inserting at least a portion of an electrode array into a cochlea, except that the action of deforming the first portion of the stimulating assembly is executed after insertion of the at least a portion of the electrode array into the cochlea. The remaining portion of the electrode array is then inserted into the cochlea. Indeed, in at least some exemplary embodiments, any order of actions that can enable the teachings detailed herein and/or variations thereof to be practiced can be utilized in at least some exemplary embodiments.

Still further, keeping in mind that in the current described exemplary methods, cochlear implant 144 or 146 or 164 (any reference to a cochlear implant of one embodiment corresponds to a reference to that of another embodiment, etc. in the interests of textual economy, provided that the art enable such, unless otherwise noted) is being implanted, in some exemplary actions of implanting the stimulating assembly includes plastically deforming a second portion of the stimulating assembly (which can have the wire or cage, etc.) so as to maintain the second portion now deformed at a second orientation due to the deformation of the second portion. In this regard, this can entail establishing bend 148. However, it is noted that this can also be executed utilizing any of the other cochlear implants detailed herein. This can be achieved via cochlear implant 146, or cochlear implant 130. That is, the various bends can be established in the same malleable structural component, just at different portions thereof.

Note further that in an exemplary method, the action of deforming the first portion of the stimulating assembly is executed before insertion of the at least a portion of the electrode array into the cochlea, and the action of deforming the second portion of the stimulating assembly is executed after insertion of the at least a portion of the electrode array into the cochlea.

Note further that in an exemplary method, the action of deforming the first portion of the stimulating assembly is executed before insertion of the at least a portion of the electrode array into the cochlea, and the action of deforming the second portion of the stimulating assembly is also executed before insertion of the at least a portion of the electrode array into the cochlea.

Note further that in an exemplary method, the action of deforming the first portion of the stimulating assembly is executed after insertion of the at least a portion of the electrode array into the cochlea, and the action of deforming the second portion of the stimulating assembly is also executed after insertion of the at least a portion of the electrode array into the cochlea.

Note further that in some exemplary embodiments, subsequent actions of deforming a third or fourth portion can be executed before and/or after the insertion of the at least a portion of the electrode array to the cochlea. Note further that previously deformed portions can be re-deformed, such as before insertion of the at least a portion of the electrode array into the cochlea and/or after insertion of the at least a portion of the electrode array into the cochlea.

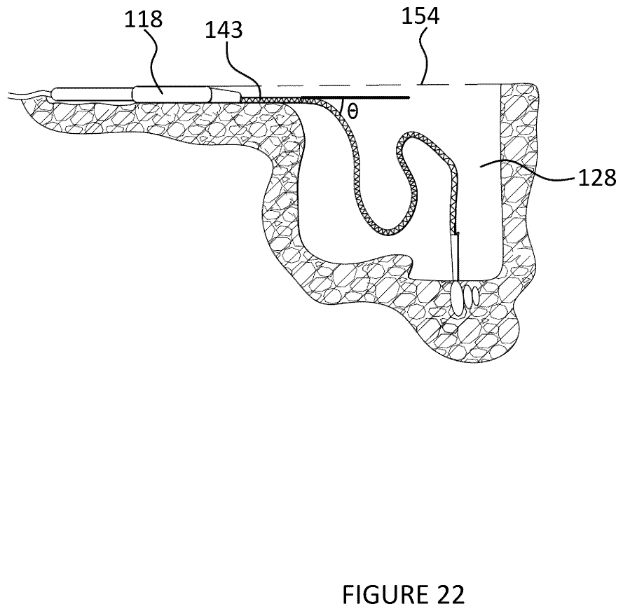

Still further, in an exemplary method, the accessed subcutaneous region need not include the channel 127 in the mastoid bone 124 of the recipient leading to the mastoid cavity 128, although in the current exemplary methods, the channel is present. In at least some of these exemplary embodiments, the mastoid cavity is part of a cavity that also includes the middle ear cavity, which combined cavity is bounded in part by a round and an oval window of a cochlea of the recipient. With this as background, the action of implanting the electrode assembly includes placing the first portion of the stimulating assembly into the artificial channel 127 such that a first sub-portion is located in the channel and a second sub-portion extends from the channel into the mastoid cavity. In this exemplary embodiment, the action of deforming the first portion of the stimulating assembly entails bending the first portion such that the second sub-portion is moved from a first orientation relative to the first sub-portion to a second orientation relative to the first sub-portion, and the plastic deformation maintains the second sub-portion at the second orientation (e.g., the orientation established by bend 147 of FIG. 17). In view of the various figures detailed herein, it can be seen that in some exemplary embodiments, the resulting second orientation is such that a longitudinal axis of the second sub-portion is at least about 45 degrees from a longitudinal axis of the first sub-portion. This angle can be represented by O in FIG. 22. In some exemplary embodiments, the resulting second orientation is such that a longitudinal axis of the second sub-portion is at least about 60 degrees from a longitudinal axis of the first sub-portion. In some exemplary embodiments, the resulting second orientation is such that a longitudinal axis of the second sub-portion is at least about 75 degrees from a longitudinal axis of the first sub-portion. In some exemplary embodiments, the resulting second orientation is such that a longitudinal axis of the second sub-portion is at least about 80 degrees from a longitudinal axis of the first sub-portion. In some exemplary embodiments, the resulting second orientation is such that a longitudinal axis of the second sub-portion is at least about 85 degrees from a longitudinal axis of the first sub-portion.

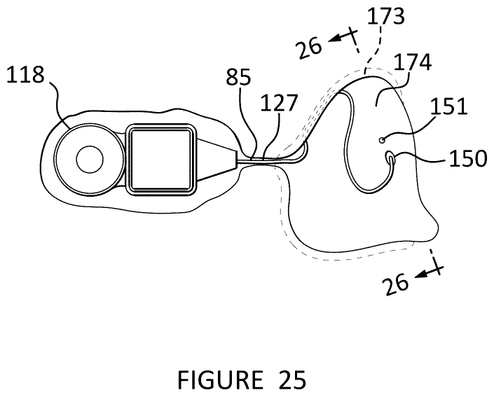

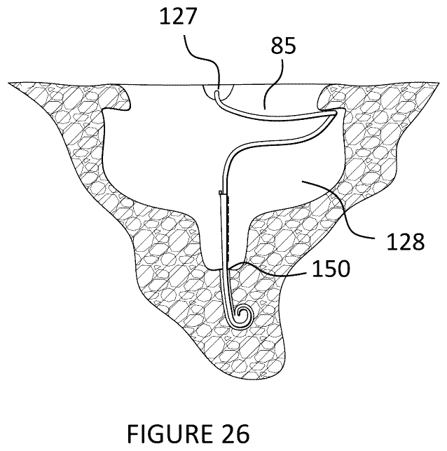

It is noted that some exemplary embodiments can have utilitarian value in that the lead assembly of the cochlear implants can be maintained in the mastoid cavity without the use of a so-called bony overhang. In this regard, FIG. 25 depicts a top view of an alternate embodiment to implanting a stimulating assembly according to the teachings detailed herein, depicting bony overhang 173, with FIG. 26 depicting the accompanying cross-sectional side view of a portion thereof. Here, unlike the mastoid cavity 128 above, mastoid cavity 174 includes bony overhang 173. In an exemplary embodiment, during the establishment of the mastoid cavity by removing bone of the mastoid bone, the surgeon or other health care professional leaves a bony overhang 173. In an exemplary embodiment, this bony overhang is utilized to maintain or otherwise provide resistance against the lead assembly 110 from springing out of the mastoid cavity 174 owing to the aforementioned elasticity detailed above or other phenomenon. In this regard, as can be seen, the lead assembly 110 can be tucked underneath the bony overhang 173. That said, in some exemplary embodiments, the various method actions detailed herein can include executing one or more or all of the methods detailed herein without placing any portion of the stimulating assembly against any bone overhanging the mastoid cavity. Corollary to this is that in an exemplary embodiment, the methods detailed herein include the action of securing a portion of the stimulating assembly located between an intracochlear electrode and a receiver/stimulator connected to the stimulating assembly in a mastoid cavity without contacting a bony overhang of the mastoid cavity. Note further, that in an exemplary embodiment, the methods detailed herein can include establishing a mastoid cavity, wherein upon the establishment of the mastoid cavity, no bony overhang is present. This is because the exemplary embodiments detailed herein can, in some instances, alleviate any utilitarian value with respect to the aforementioned bony overhang.

It is noted that the aforementioned method actions are but exemplary. Other exemplary methods include other method actions and/or variations of the actions detailed herein. Any method that can enable the teachings detailed herein and/or variations thereof to be practiced can be utilized in at least some exemplary embodiments. Any of the configurations/orientations detailed herein of any other components of the disclosed cochlear implants correspond to a disclosure of a method for placing the stimulating assembly is into those configurations.

Some alternate configurations of the malleable structures will now be detailed.

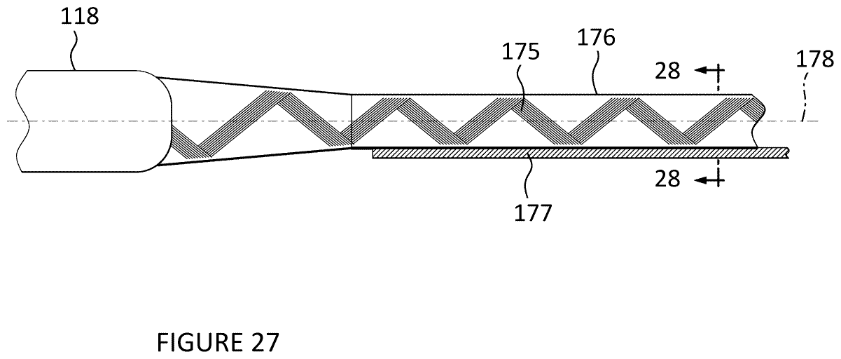

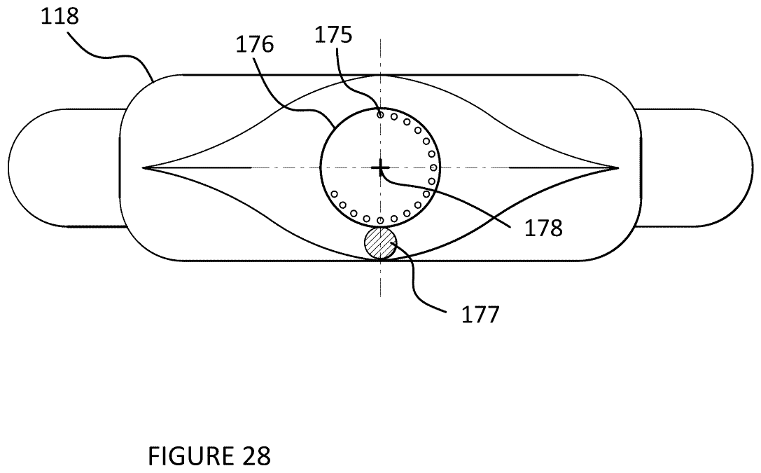

FIG. 27 presents a side view of an exemplary alternate embodiment, and FIG. 28 depicts a cross-sectional view thereof. Briefly, it is noted that as with the embodiment of FIG. 9, there exists an elongate stimulation assembly of a cochlear implant (or other implantable stimulation device), comprising an intra-cochlear portion including an array of electrodes, and lead wires 175 (only one is shown, in FIG. 27, for purposes of clarity, but more are shown in FIG. 28) extending from the intra-cochlear region in electrical communication with the array of electrodes, the lead wires being located in an elongate lead body 176. As with the embodiment of FIG. 9, the elongate stimulation assembly includes a malleable component 177 extending in an elongate manner at least partially along with the lead wires 175. As with FIG. 9, the malleable component 177 is located closer to an outer surface of the lead body 176 than at least one of the lead wires 175.

It is further noted that in an exemplary embodiment, the malleable component is located the same distance from an outer surface of the lead body as at least one of the lead wires.

In the exemplary embodiment of FIG. 27, the malleable component 177 is also a metallic wire. However, whereas the malleable component 177 was embedded in the body 176 (e.g., embedded in the silicone in which the wire leads were also embedded), or otherwise located within a conduit forming the lead body, in this embodiment, the malleable component 177 is located outside the body/outside the silicone. In an exemplary embodiment, the malleable component 177 is bonded to the body 176. In an exemplary embodiment, the malleable component 177 is mechanically attached in some manner to the body 176. By way of example only and not by way of limitation, surgical sutures or metal bands or silicone bands, or plastic bands can extend about the body 176 while also encompassing the malleable component 177. Any arrangement that can enable sufficient portions of the body 176 to be coupled to the malleable component 177 so that the teachings detailed herein and/or variations thereof can be enabled and practiced can be utilized in at least some exemplary embodiments. In an exemplary embodiment, by way of example only and not by way of limitation, the malleable component or otherwise the stiffening material located outside the body/outside the silicone, could be used, in some exemplary embodiments, as an extra-cochlear electrode. In an exemplary embodiment, the entire malleable component could be used as such, at least the portions thereof that are located outside the body/silicone. That said, in an alternate embodiment, the malleable component could be insulated or otherwise provided with a coating, but a portion of this insulation and/or coating could be not present, thus exposing the malleable component within the coating to the ambient environment so that this exposed portion can be utilized as the extra cochlear electrode. In a similar vein, in an exemplary embodiment, the portion of the malleable component expose the ambient environment can be utilized as the return electrode. Accordingly, in an exemplary embodiment, the malleable component could extend to the receiver stimulator of the implanted cochlear implant. That said, in an alternate embodiment, the malleable component may and prior to extending to the receiver your stimulator component, but the malleable component can still be in electrical communication with the receiver stimulator component. For example, a separate electrical lead wire separate from the stimulating leads could extend from the receiver stimulator, but instead of extending to the electrodes of the electrode array, could extend to the malleable component.

Briefly, it is noted that the wire leads 175 are arrayed in a helical structure, as seen. To be clear, while the embodiment of FIG. 27 depicts the malleable component 177 located on the outside of the body 176, an alternate embodiment can utilize the helical wires along with the malleable component 177 embedded in the body 176. That is, the current application has been presented in an efficient matter to convey the various concepts, but it is to be understood that some embodiments entail combining concepts of one embodiment with concepts of another embodiment. Indeed, some exemplary embodiments entail a combination of one or more features from one embodiment with one or more features of another embodiment unless otherwise indicated or otherwise impractical to do so.

In view of FIGS. 27 and 28, it can be seen that in some exemplary embodiments, the extra-cochlear portion of the stimulating assembly includes a lead body, and the malleable component is a metal wire located completely outside the lead body. It is further noted that in some exemplary embodiments, the malleable structure is located completely away from the lead body in an offset manner. Connection brackets or the like can be utilized to place the lead body into mechanical connection with the malleable structure (e.g., posts can be located every millimeter or so along the length of the malleable structure, etc.).

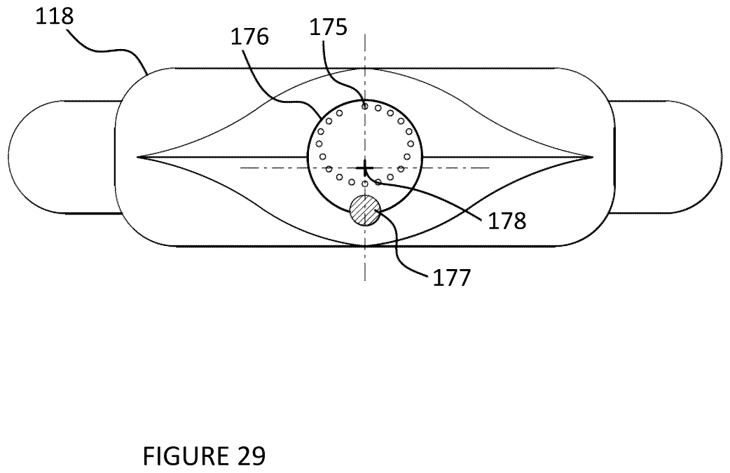

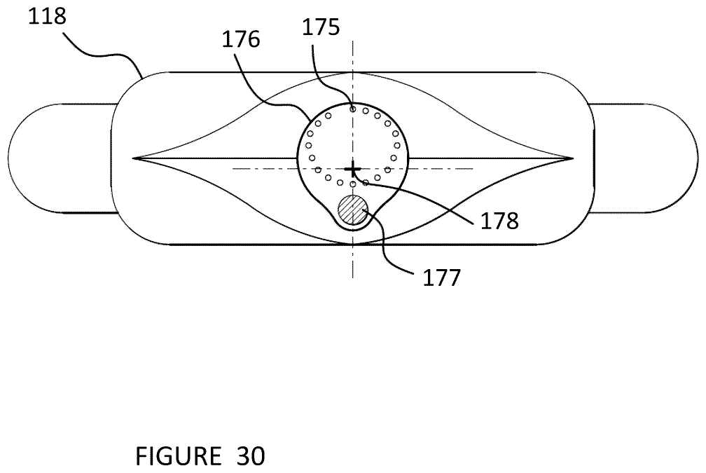

In view of FIGS. 27 and 28, an exemplary embodiment includes a stimulating assembly where the extra-cochlear portion includes a lead body 176, and the malleable component 177 is a metal wire located at least partially external to the lead body 176 attached to the lead body 176. FIG. 29 depicts another example of such an embodiment, where the malleable component 177 is partially embedded in the body 176. Corollary to the above, exemplary embodiments include embodiments where the malleable component is a metal wire located at least partially external to the lead body and attached to the lead body. FIG. 30 depicts full embedding, where the body includes extra material to encapsulate the malleable structure. It is noted that the longitudinal axis 178 has been “moved” relative the longitudinal axis of 139 to account for the fact that the overall outer diameter of the stimulating assembly has been extended, thus the center thereof has been moved downward owing to the placement of the malleable component outside the body.

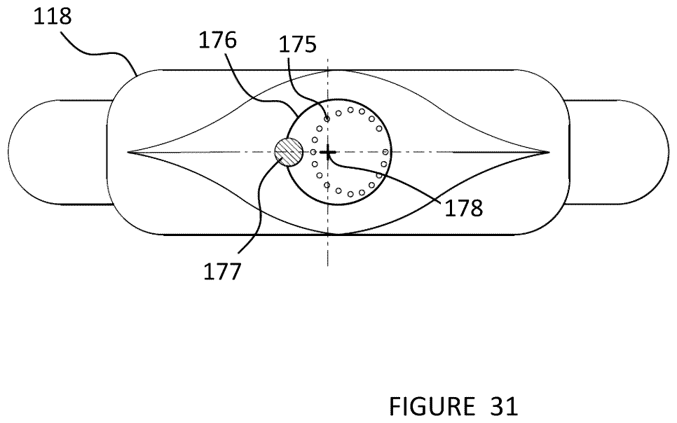

FIG. 31 depicts an alternate embodiment of the arrangement of FIG. 29, where the lead wires 175 and malleable component 177 have been rotated 90° in a clockwise direction relative to that which is the case in FIG. 29. That is, the malleable component 177, instead of being located at the bottom of the body of 176, is located to the side of the body 176 (relative to the frame of reference where the bottom corresponds to the portion closest to the bottom of the channel formed in the mastoid bone, etc.). It is further noted that in at least some exemplary embodiments, the malleable component 12 can be located on the other side of the longitudinal axis 178 relative to that which is depicted in FIG. 31. Note further, that in an exemplary embodiment, the various components could have been rotated 180° relative to that which is the case depicted in FIG. 29. That is, the malleable component 177 can be located on the top of the body 176. Note further, other orientations of the malleable component and/or other components can be utilized (e.g., the malleable component could be located at the 7:30 position, the 10:30 position, or any other position that can have utilitarian value).

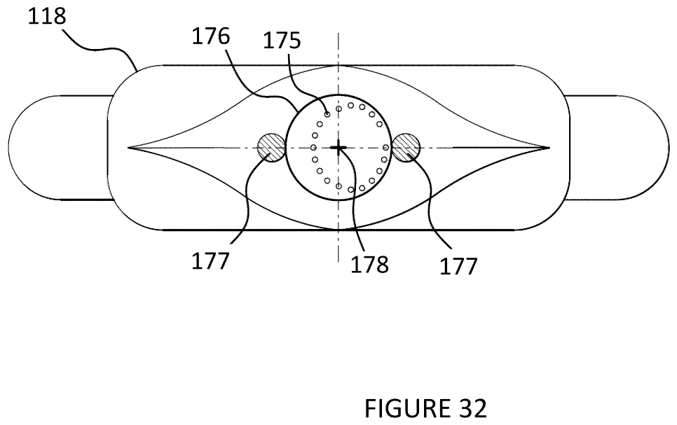

FIG. 32 presents yet another alternative embodiment, where two malleable components 177 are located on opposite sides of body 176. While this dual-malleable component embodiment is depicted with respect to the malleable components being located at the 3 o'clock position and that the 9 o'clock position, in alternative embodiments, the malleable components can be located at the 12 o'clock position and at the 6 o'clock position or any other positions for that matter. Note further that the positioning need not necessarily be symmetric about the longitudinal axis 178. Additionally, three or more malleable components can be utilized. Further, the malleable components need not necessarily be identical. In some embodiments, one of the malleable components can have a larger diameter than the other malleable component. Moreover, it is noted that the material properties of one malleable component can be different than the material properties of another malleable component (e.g., one could be made of one material and one could be made from another material). Additionally, one of the malleable components could be at least partially embedded in the body 176, while one other of the malleable components could be completely outside the body 176. Still further, in an exemplary embodiment, both of the malleable components can be at least partially within the body 176, and in some embodiments, all of the malleable components are completely within body 176. Again, any arrangement that can enable the teachings detailed herein and/or variations thereof to be practiced can be utilized in at least some embodiments.

In view of FIG. 32, in an exemplary embodiment, there is a cochlear implant that is configured to prevent the movement of the at least a portion of the lead assembly due to the elasticity via a plurality of malleable wires co-located with the lead assembly. In this exemplary embodiment, the plurality of wires are located outside the lead assembly extending parallel thereto and extend through a same plane that is normal to a local direction of extension of the lead assembly.

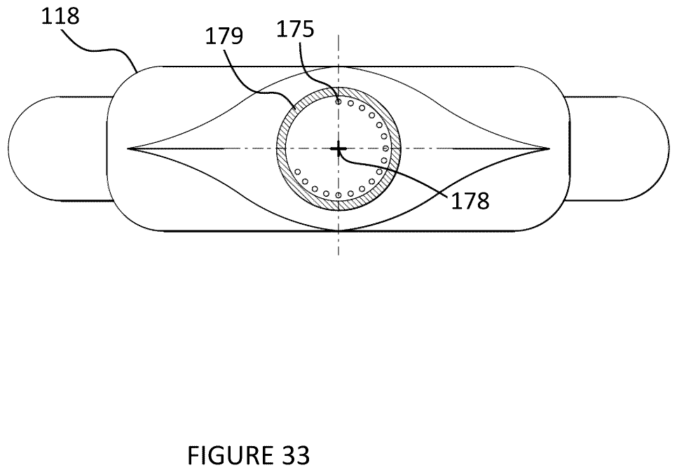

FIG. 33 depicts another alternate embodiment, where the malleable structural component is a tube 179 that encompasses the body of the lead assembly. In an exemplary embodiment, the tube 179 is configured so as to avoid collapsing upon bending. It is noted that while the embodiment of FIG. 33 depicts the tube 179 on the outside of the body of the lead assembly, in an alternate embodiment, the tube can be implanted in the body of the lead assembly.

Thus, in an exemplary embodiment, there is a cochlear implant configured to prevent the movement of the at least a portion of the lead assembly due to the elasticity via a structure co-located with the lead assembly, wherein the structure is a tube extending about the lead apparatus.

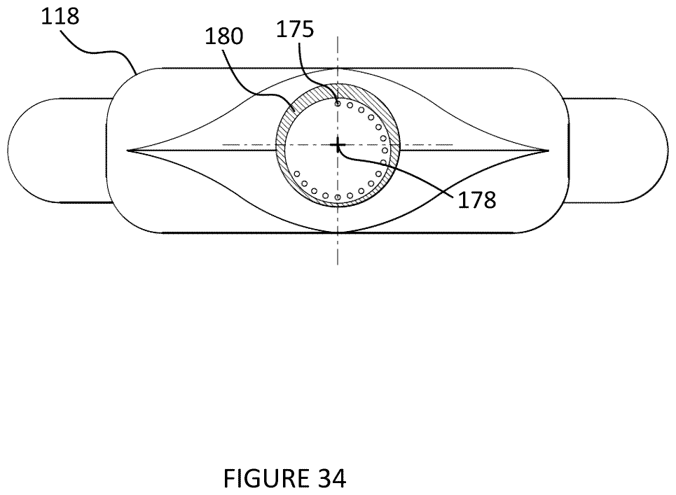

Still further, in an exemplary embodiment, implantation of an implant utilizing the arrangement of FIG. 33 can entail a method of bending the stimulating assembly by an amount limited to the amount beyond which crimping and/or collapsing of the tube 179 would result. Still further, in an exemplary embodiment, the tube can be asymmetrical about the longitudinal axis 178. Such an exemplary embodiment can be seen in FIG. 34, where the wall thickness at the bottom of tube 2010 is thinner than the wall thickness at the top of tube 2010. In an exemplary embodiment, this can avoid the aforementioned collapsing and/or crimping, or at least mitigate the effects thereof. Indeed, in an exemplary embodiment, a portion of the tube can be configured to separate from another portion (e.g., split), so as to avoid the collapsing/crimping phenomenon. Still further, in an exemplary embodiment, the tube includes a break zone configured to separate upon the bending. In an exemplary embodiment, the break zone is an area of relatively thinner wall thickness relative to the wall thicknesses of other components of the tube. That said, in an alternate embodiment, the tube can have a slit therein running parallel to the longitudinal axis 178 so that upon bending, the portions forming walls of the slit can expand from one another so as to avoid or otherwise alleviate the aforementioned collapsing and/or crimping.

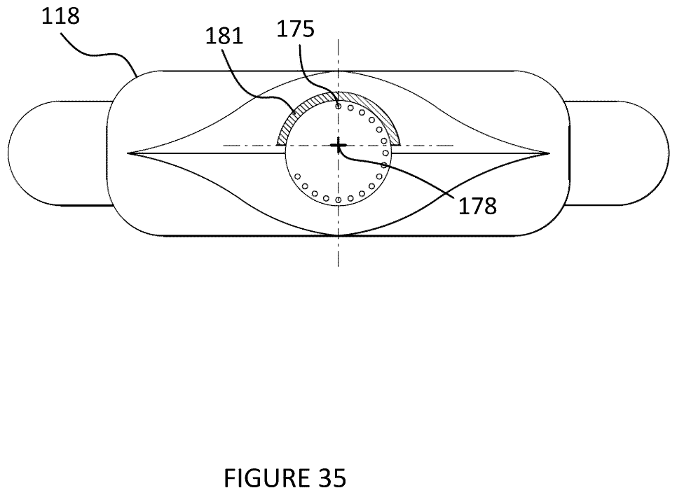

Corollary to the above is that a portion of a tube structure can be utilized/a C-shaped structure can be utilized as the malleable component. In this regard, FIG. 35 depicts another exemplary embodiment of a malleable structure 181 in the form of a C-shaped elongate component (elongate in the direction of the longitudinal axis 178). As can be seen, the malleable structure 177 extends about a portion of the body of the lead assembly. While the embodiment depicted in FIG. 35 depicts a C-shaped structure that subtends an angle of about 170° about the longitudinal axis 178, in some alternative embodiments, the angle subtended is less than 170° or greater than 170° (e.g., about 145°, about 270°, etc.). Any arrangement that can enable the teachings detailed herein and/or variations thereof to be practiced can be utilized in at least some exemplary embodiments.

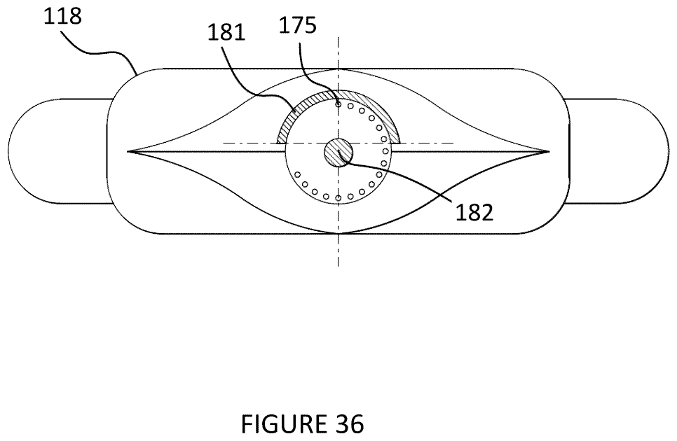

FIG. 36 depicts an alternative embodiment where there is both an outside malleable structure 181 and a malleable structure 182 encapsulated in the body of the lead assembly. Thus, an exemplary embodiment entails a plurality of malleable structures, where one of the structures is located further away from the longitudinal axis of the lead assembly than the lead wires. While some exemplary embodiments, such as that depicted in FIG. 36, are such that the body completely separates the malleable structure located outside the body from the malleable structure located inside the body, in some alternate embodiments, the structures are connected to each other by another malleable structure and/or a rigid structure and/or a flexible structure other than silicone, or other structure establishing the bulk of the body of the lead assembly.

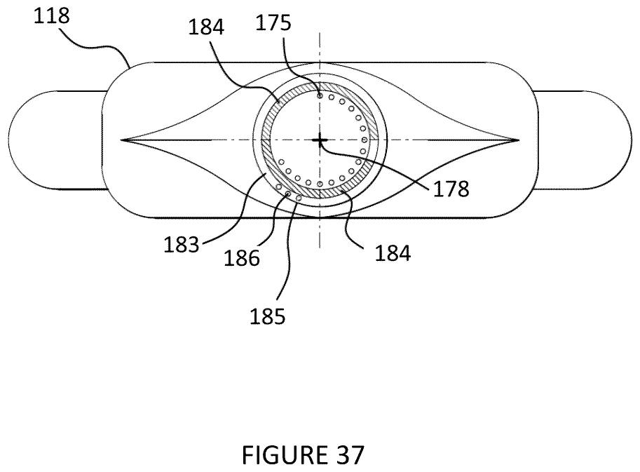

As noted above, some embodiments can utilize the tube structure embedded in the body of the lead assembly. In this regard, FIG. 37 depicts a malleable structure in the form of a tube 184 completely embedded in the body 185 of the lead assembly. Note further that in this exemplary embodiment, the body 185 includes a portion inside the tube 184 and a portion located outside the tube 184, annotated as 183. In an exemplary embodiment, the portion 183 is also made of silicone. In an exemplary embodiment, there are holes or passages located through the walls of the tube 184 such that when the silicone body is molded, the silicone on the outside of the tube 184 is in the silicone communication with the silicon on the inside of the tube 184. Note further that in this exemplary embodiment, there are lead wires 186 located on the outside of the tube 184 as well as lead wires 175 located inside the tube 184. Thus, while in this embodiment, the malleable structure (tube) 184 is still located away from the longitudinal axis 178 a greater distance than at least one of the lead wires 175, and is also located closer to the surface of the body 185 and at least one of the lead wires 175, there are other lead wires that are located closer to the surface of the body than the malleable structure 184, and located further away from the longitudinal axis 178 then the malleable structure 184.

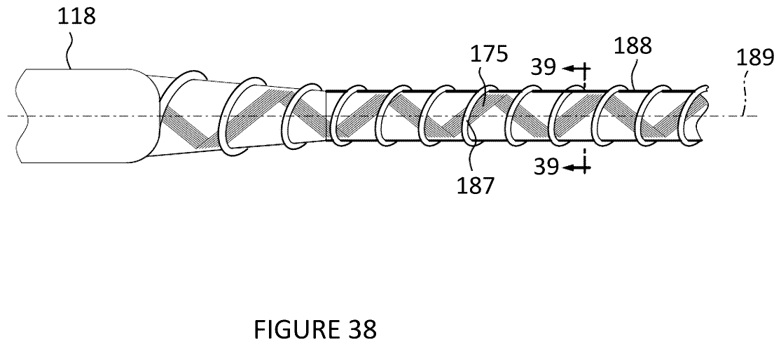

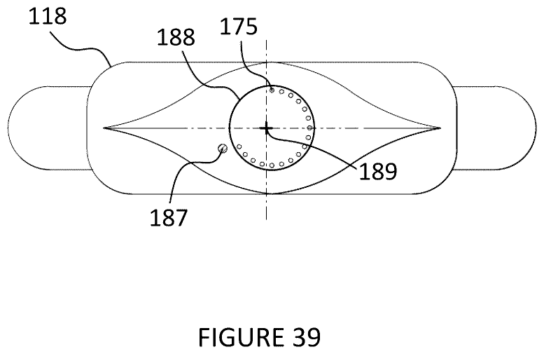

FIGS. 38 and 39 depict an alternate embodiment where the malleable component is a helical structure. More particularly, as can be seen, structure 187 is a malleable helical structure that extends about the outer surface of the body 176 of the lead assembly. As can be seen, in this exemplary embodiment, the malleable helical structure is wrapped around at least some of the lead wires 175, which are also in a helical structure.

In the embodiments of FIGS. 38 and 39, the helical structure extends about the lead body 176 at least partially external to the lead body. In the embodiments of these figures, the helical structure extends fully externally to the lead body. Here, the helical structure is attached to the surface of the lead body 176 as can be seen. The attachment can be through bonding or any suitable mechanical structure. That said, in an alternate embodiment, the helical structure itself can be utilized to hold itself to the lead body 188. That is, because the helical structure winds about the lead body 176, it will hold itself in place. Along these lines, in an exemplary embodiment, the helical structure that extends about the lead body is configured such that the lead body can move locally relative to the helical structure. That is, the body can move by at least some amount in the longitudinal direction (e.g., in the direction of longitudinal axis 189) and/or in the lateral direction by at least some amount relative to the helical structure 187. In at least some exemplary embodiments, this can have utilitarian value where helical structure 187 prevents the global movement of the lead body 188. That is, while the lead body 188 can move locally (e.g., within the confines of the helical structure 187), the lead body cannot move globally (cannot change the trajectory/orientation of the helical structure 187, and thus cannot move globally).

It is noted that while the aforementioned movement features are disclosed with respect to a helical structure, in some alternative embodiments, the aforementioned movement features can also be achieved utilizing some other structures. By way of example only and not by way of limitation, the lead body can be flexibly attached to the malleable wires detailed herein so that limited local movement can occur, but no global movement can occur.

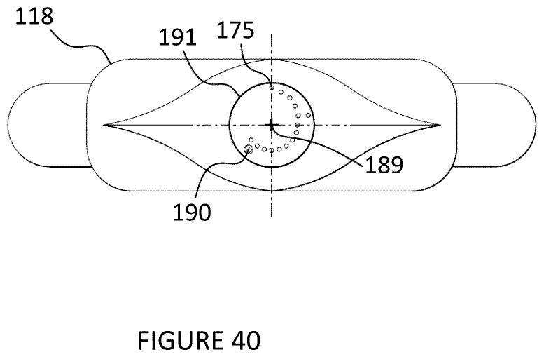

While the embodiments of FIGS. 38 and 39 depict the helical structure located outside the lead body, in some alternate embodiments, the helical structure can be embedded within the body. In this regard, FIG. 40 depicts a helical structure 190 embedded within the body 191 of a stimulating assembly.

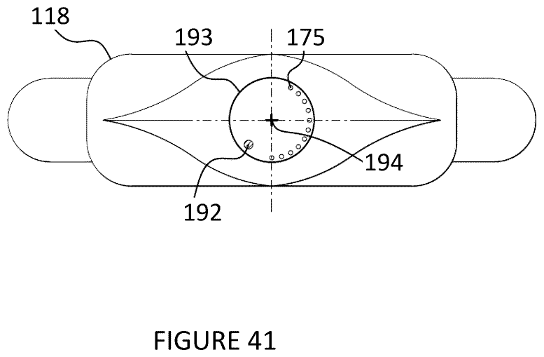

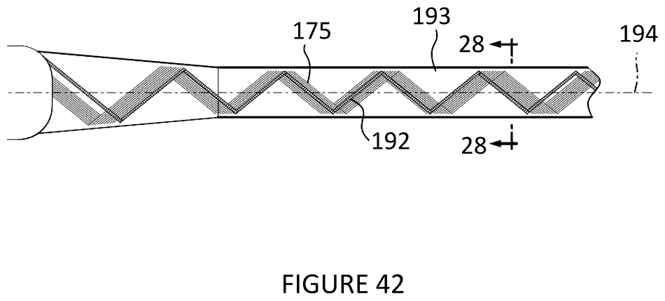

FIGS. 41 and 42 depict an alternate embodiment where the malleable component is again a helical structure. More particularly, as can be seen, structure 192 is a malleable helical structure that extends inside the body 193 of the lead assembly. As can be seen, in this exemplary embodiment, the malleable helical structure is a first helix, while the lead wires 175 are also in a second helix, where the first helix and the second helix form a double helix. As can be seen, the outer diameter of the first helix is about the same as the outer diameter of the second helix.

In the embodiments of FIGS. 41 and 42 the helical structure extends within the lead body 193, about the longitudinal axis 194. In the embodiments of these figures, the helical structure extends fully internally to the lead body 193.

It is further noted that concomitant with the embodiments presented above where two or more malleable structures are utilized, in an exemplary embodiment, the cochlear implants according to at least some exemplary embodiments are configured to prevent the movement of the at least a portion of the lead assembly due to the elasticity via a malleable double helix assembly co-located with the lead assembly. That is, in some embodiments, two separate structures both having separate helix shapes can be utilized. That said, in an alternate embodiment, the double helix can be part of a single structure, where two sub-components have the helix structure, and are connected together by another sub-component. Corollary to this is that the embodiments presented above where the malleable components are two separate structures can also be practiced where the malleable components are part of substructures of the same structure connected together by a third structure.

Note further that in some exemplary embodiments, a double helix structure can be utilized where the lead wires form one of the helixes, and the malleable component forms another one of the helixes. The two helixes can be equidistant from the longitudinal axis of the lead assembly, or, in an alternate embodiment, one of the helixes can be closer to the longitudinal axis than the other of the helixes. In an exemplary embodiment, the malleable helix is located further away from the longitudinal axis, and/or closer to the outer surface of the body of the lead assembly then the helix formed by the lead wires.

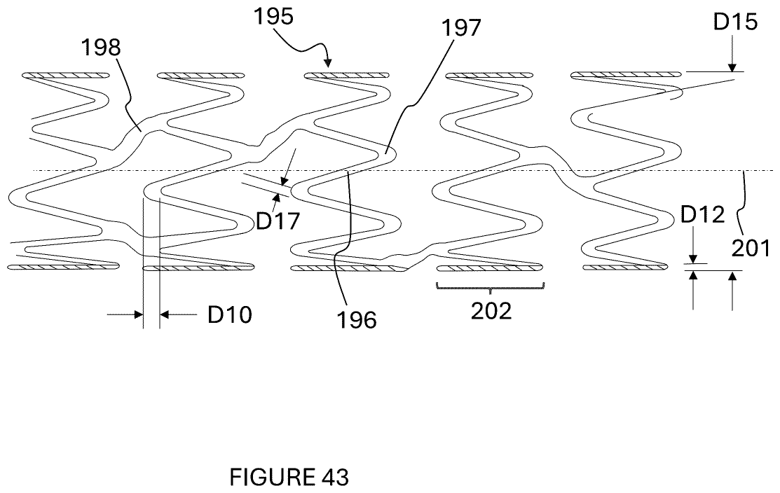

FIG. 43 shows a cross-section of a cage 195 that can correspond to cage 165 detailed above, by way of example only and not by way of limitation (which cross-section is taken on a plane lying on and coincident to a longitudinal axis of the cage). Consistent with the basic concepts underlying all disclosure herein, the embodiment of FIG. 43 can be utilized in the embodiments of the cage 165 above, and/or can be utilized instead of than or in addition to the malleable component as detailed above and/or below, all in the interest of actual economy, and vice versa. In this exemplary embodiment, the cage 195 includes segments 202 which are linked to each other by way of segment connector 198. In an exemplary embodiment, there are at least and/or equal to and/or greater than 2, 3, 4, 5, 6, 7, 8, 9, 10, 11, 13, 14, 15, 16, 17, 18, 19, 20, 21, 22, 23, 24, 25, 26, 27, 28, 29 or 30 or more or any value or range of values therebetween in 1 increment segments per cage, and there can be at least and/or equal to and/or more than 1, 2, 3, 4, 5, 6, 7, 8, 9, 10, 11, 13, 14, 15, 16, 17, 18, 19, 20 or 21 any value or range of values therebetween in 1 increment cages per stimulating assembly depending on the arrangement and otherwise the embodiment. In an exemplary embodiment, there are at least and/or equal to and/or greater than 1, 2, 3, 4, 5, 6, 7, 8, 9, 10 or more or any value or range of values therebetween in 1 increment segment connectors 198 between a given segment and another segment, and the number of segment connectors need not be the same in a given cage. In the embodiment shown in FIG. 43, there are more segment connectors 198 on the left side of FIG. 43 than that which is the case on the right side of FIG. 43. In an exemplary embodiment, there are more segment connectors located at the proximal portions of the cage than the distal portions of the cage when applied to the stimulating assembly. In other embodiments, the opposite is the case. Also, there can be embodiments where the number of segment connectors 198 are greater in the middle or thereabouts of the cage relative to the ends of the cage. Note also that the number of segment connectors can vary along the length of the cage to be greater and than and/or less and then greater and then less, etc. relative to the number of segment connectors to the left and right of a given segment along the length of the cage. In an exemplary embodiment, the more segment connectors, the greater the axial strength of the cage, while in other embodiments, the less segment connectors, the lower the axial strength of the cage. In an exemplary embodiment, the greater the number of segment connectors, the greater the axial stiffness, while in other embodiments, the fewer segment connectors, the lower the axial stiffness. In an exemplary embodiment, the more segment connectors, the greater the flexural (bending) stiffness, while in other embodiments, the fewer segment connectors, the lower the flexural (bending) stiffness. Any arrangement of connectors that can implement the teachings detailed herein that can have utilitarian value can be implemented in some embodiments. In an embodiment, the design increase in segment connectors from 1 or 2, or 3, or 4 or 5 or 6 or 7 or 8 or 9 or 10 or 11 or 12 to 2 (in which case the design increase is 1, 3, 4, 5, 6, 7, 8, 10, 11, 12, 13, 14 or 15 or any value or range of values therebetween in 1 increment between segments results in an increase of at least 5, 10, 15, 20, 25, 30, 35, 40, 50, 60, 70, 80, 90, 100, 125, 150, 175, 200, 250, 300, 350, 400, 450 or 500% or more or any value or range of values therebetween in 1% increments of any one or more of the just noted qualities, all other things being equal, and vis-a-versa (a decrease from any of the numbers results in a decrease by any of the just-noted percentages, in the interests of textual economy).

Note also that the tempering of the material and/or the material composition can be used to have different axial strengths for a given geometric design. Different, geometries/dimensions can be used to obtain different axial strengths. A combination of this can be implemented in some embodiments.

As seen, a given cage segment is made up of stringers 196 that are connected to each other via elbows 197. In an exemplary embodiment, the stringers (sometimes referred to as struts-here, this is a body that extends in a longitudinal direction and has a generally uniform thickness and width along that direction, but as seen, in some embodiments the width varies, and in some embodiments (not shown in the figures, but can be the case), the thickness varies, all along the longitudinal direction) 196 extend upward from a plane that is lying on the longitudinal axis 201 and coincident there with which extend at an angle that is less than, greater than and/or equal to 5, 10, 15, 20, 25, 30, 35, 40, 45, 50, 55, 60, 65, 70, 75 or 80 degrees or any value or range of values therebetween in 1° increments from that plane, where the plane extends through a geometric center of the given stringer/strut associated with the given angle under calculation. This angle is measured between the longitudinal axis of the given stringer 196 and the given plane.

In an exemplary embodiment, there are at least and/or equal to and/or greater than 2, 3, 4, 5, 6, 7, 8, 9, 10, 11, 13, 14, 15, 16, 17, 18, 19, 20, 21, 22, 23, 24, 25, 26, 27, 28, 29, 30, 35 or 40 or more or any value or range of values therebetween in 1 increment stringers per segment. In an exemplary embodiment, a length of a given stringer is less than and/or equal to and/or greater than 5, 6, 7, 8, 9, 10, 11, 13, 14, 15, 16, 17, 18, 19, 20, 21, 22, 23, 24, 25, 26, 27, 28, 29, 30, 35, 40, 45, 50, 55, 60, 65, 70, 80, 90, 100, 110, 120, 130, 140, 150, 160, 170, 180, 190, 200, 220, 240, 260, 280 or 300×10⁻² mm (that is, any of the just noted numbers multiplied by 10⁻², in the interests of textual economy) or more or any value or range of values therebetween in 0.01 mm increments. These values can also be the case for the segment connectors 198. The values need not be the same between segments or in segments, etc.

The length of the cages can be any of the lengths of the malleable wire structures detailed herein.

In an embodiment, D10 of the elbows 197 is less than and/or equal to and/or greater than 5, 6, 7, 8, 9, 10, 11, 13, 14, 15, 16, 17, 18, 19, 20, 21, 22, 23, 24, 25, 26, 27, 28, 29, 30, 35, 40, 45, 50, 55, 60. 65, 70, 80, 90, 100, 120, 140, 160 or 200×10⁻² mm or more or any value or range of values therebetween in 0.01 mm increments. In an embodiment, D12 can be less than and/or equal to and/or greater than 2, 2.5, 3, 3.5, 4, 4.5, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 80, 90, 100, 120, 140, 160 or 200×10⁻² mm or more or any value or range of values therebetween in 0.01 mm increments. Note that in an exemplary embodiment, the thickness D12 of the stringer is also the thickness of the elbows 197 and or the segment connectors 198, although in other embodiments, the thicknesses could be different for those elements and the thicknesses could be any of the values just detailed for D12 in the interest of textual economy. In an exemplary embodiment, the diameter of the cage 195, D15, normal to the longitudinal axis of the cage 201, can be less than and/or equal to and/or greater than 1.5, 2, 2.5, 3, 3.5, 4, 4.5, 5, 6, 7, 8, 9, 10, 11, 13, 14, 15, 16, 17, 18, 19, 20, 21, 22, 23, 24, 25, 26, 27, 28, 29, 30, 35, 40, 45, 50, 55, 60, 65, 70 or 80×10⁻¹ mm or more or any value or range of values therebetween in 0.01 mm increments. In an exemplary embodiment, the diameter D15 need not be the same along the length of the cage 195. In an exemplary embodiment, different segments can have different diameters and the diameters of the different segments can be any of the values just detailed by way of example only and not by way of limitation. In an embodiment, D17, the width of a stringer 196, can be less than and/or equal to and/or greater than 11, 13, 14, 15, 16, 17, 18, 19, 20, 21, 22, 23, 24, 25, 26, 27, 28, 29, 30, 35, 40, 45, 50, 55, 60, 65, 70, 80, 90, 100, 120, 140, 160 or 200×10⁻² mm or more or any value or range of values therebetween in 0.01 mm increments.

Controlling the value of D12 and/or D17 and/or any of the other dimensions of the cage can vary the deformability of the cage/given stringer. In an exemplary embodiment, by controlling for example the aspect ratio of a stringer, a deformability can be achieved which can avoid cracking relative to that which would otherwise be the case. In an embodiment, biocompatible materials and/or plastically deformable materials can be utilized to make the cage. In an embodiment, the cage is made of material such as, for example, stainless steel and/or platinum and/or palladium and/or MP35N.

The value for D10 can be different to obtain different strengths. In this regard, a wider value of D10 relative to a lower value can be used to obtain a wider peak focus strain to reduce recoil relative to that which would otherwise be the case.

Embodiments can include a cage that has expandable rings joined by links. In other embodiments, other cage structures can be utilized.

In an embodiment, the designs of the cage are provided to manage, including reduce and/or minimize, spring back, relative to that which would otherwise be the case with another design. More specifically, the utilization of hinge points, such as the elbows 197, in the various designs, can be implemented so that these hinge points undergo strains that are targeted to achieve a desired plastic deformation. These hinge points can be designed to undergo relatively large strains relative to other components of the cage at least, during bending, which strains produce sufficient local plastic deformation, which can reduce and/or minimize spring back. In an embodiment, the hinge points can be designed (and thus are designed) so as to reduce a likelihood that the bending will result in cracking or fracture, or at least such that would be considered a failure mode. For example, larger values of D10 will result in larger strains during bending relative to values that are smaller. The resulting plastic deformation is sufficiently significant so as to achieve spring back qualities/performance characteristics that are utilitarian for a given implementation. Accordingly, the values of D10 can be implemented that are different along the length of the cage 195 depending on where spring back is a concern and where such is not a concern. For example, a given cage could be of a length where parts of the cage are simply present to connect one portion of the cage to the other portion of the cage. This middle portion for example, would be in a location where spring back is less of a concern than that which would be the case at other portions. Accordingly, a lower value of D10 can be utilized in this portion relative to that which is the case for the other portions, where spring back could be more of a concern. In fact, in some embodiments, it could be that there is a desire to not have the significant plastic deformation or otherwise to minimize spring back, because it is undesired to have the cage plastically deformed at a given location along the length of the stimulating assembly. Of course, in some embodiments, the cage could simply not be present at those locations so that the stimulating assembly remains a plastically deformable. Embodiments can thus have resistance to spring back after bending. Embodiments can also provide resistance to radial compressive forces.

In some exemplary embodiments, a feature or the cage is the ability to provide bending without collapsing and/or kinking. This can be achieved by, for example, the specific placement of the segment connectors 198 and/or structuring the connectors with a given geometry/dimensions and/or structuring the segments 202 in a specific manner. By arranging the segment connectors at desired locations with respect to the radial location about the longitudinal axis of the stimulating assembly, collapsing and/or kinking can be avoided or otherwise the likelihood of such can be reduced relative to that which would otherwise be the case.

In view the above, it can be seen that in at least some exemplary embodiments, there are cage structures that are “tuned” to achieve a desired responsiveness of the stimulating assembly.

It is noted that while the embodiments detailed herein focus on the cage structure being located within the stimulating assembly, or otherwise beneath the outer surface of the body 120, in other embodiments, this may not necessarily be the case. For example, in an exemplary embodiment, the cage structure could be located on the outside of the body 120. As will be described in greater detail below, in an exemplary embodiment, the cage structure can be expanded and then the body 120 of the elongate simulating assembly can be inserted into the cage, and then the cage can be contracted around the body so as to establish an interference fit or at least a slip that they are about. That said, a clearance fit could be utilized between the cage and the silicone of the body 120. A slip fit could instead be utilized.

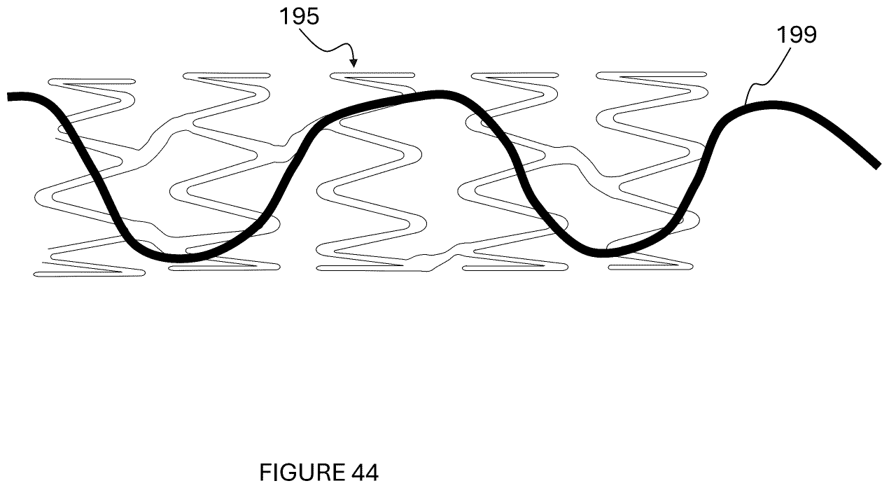

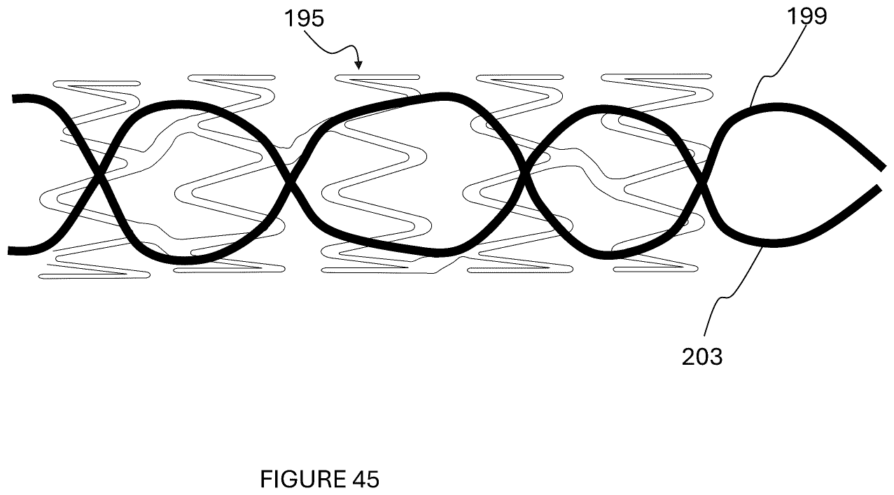

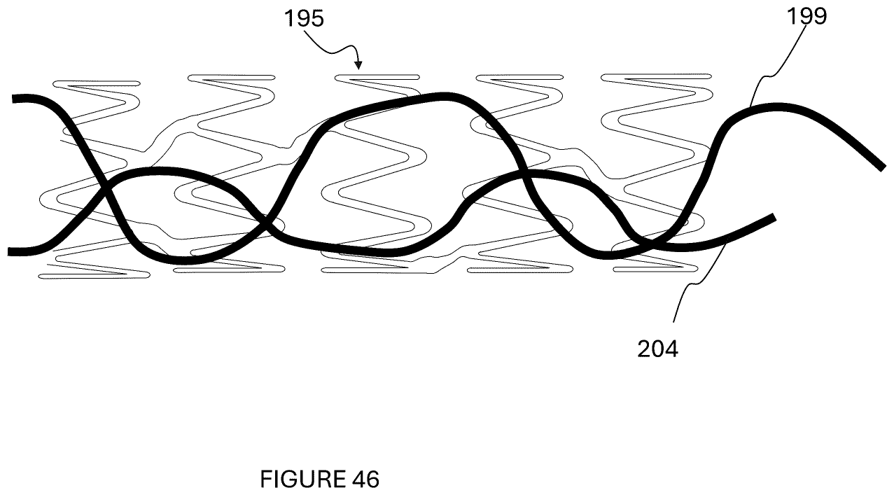

As detailed above, embodiments include the implementation of lead wires that extend inside the cage 195. FIG. 44 presents an exemplary embodiment of a wavy (or serpentine) lead wire 199. This can have utilitarian value with respect to providing sufficient “slack” of the lead wires 199 to accommodate bending and/or to accommodate a scenario where the bending might pinch/break the lead wires. The wavy (or serpentine) lead wire reduces the likelihood that a lead wire will the frayed or otherwise severed or otherwise pinched during implantation of the implantable device and/or handling of the implantable device prior to implantation and/or during manufacturing and/or after implantation. Again, in embodiment, a wavy or serpentine pattern is utilized, but while in other embodiments, different patterns can be utilized. In some embodiments, a zigzag pattern can be utilized. Note that in an embodiment, a given lead wire can have two or more or all of the given patterns disclosed herein at different longitudinal locations thereof. Any arrangement of providing slack for the lead wires relative to the length of the stimulating assembly that can enable the teachings detailed herein can be utilized in at least some exemplary embodiments. FIG. 44 shows a single lead wire, but there could be other(s) behind the wire 199 that are eclipsed by what is shown. In some embodiments, where there are multiple lead wires, some and/or all of the lead wires will have the same general pattern. In an exemplary embodiment, the lead wires 199 can represent 2 or 3 or 4 or 5 or 6 or 10 or 15 or 20 or 30 or 50 or more or any value or range of values in 1 increments lead wires or all of the lead wires or some of the lead wires. In an exemplary embodiment, the lead wires will parallel each other with respect to location in and out of the plane of FIG. 44 (hence why wire 199 could eclipse other wire(s). Conversely, FIG. 45 shows an example where there is a 2^{nd} lead wire 203 that follows a wavy (or serpentine) pattern that is the inverse of that of wire 199. Wire 203 can represent 2 or 3 or 4 or 5 or 6 or 10 or 15 or 20 or 30 or 50 or more or any value or range of values in 1 increments lead wires, and as is the case with respect to wire 199, the lead wires can be parallel with each other in some embodiments with respect to location in and out of the plane of FIG. 45. FIG. 46 shows another embodiment where there is a lead wire 204 that has the wavy (or serpentine) pattern, but has a “amplitude” and a “phase.” In some embodiments, some wires can have the same amplitude with different phase to one or two or three or four or more other wires and vice versa depending on the embodiment.

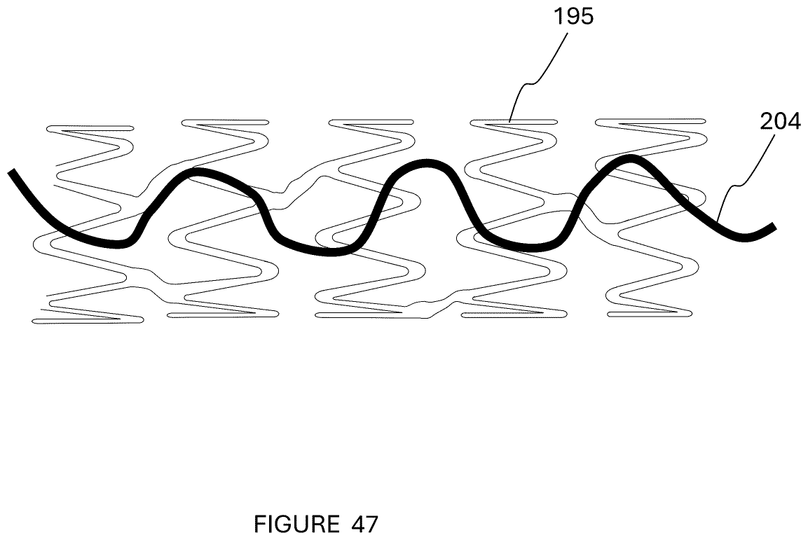

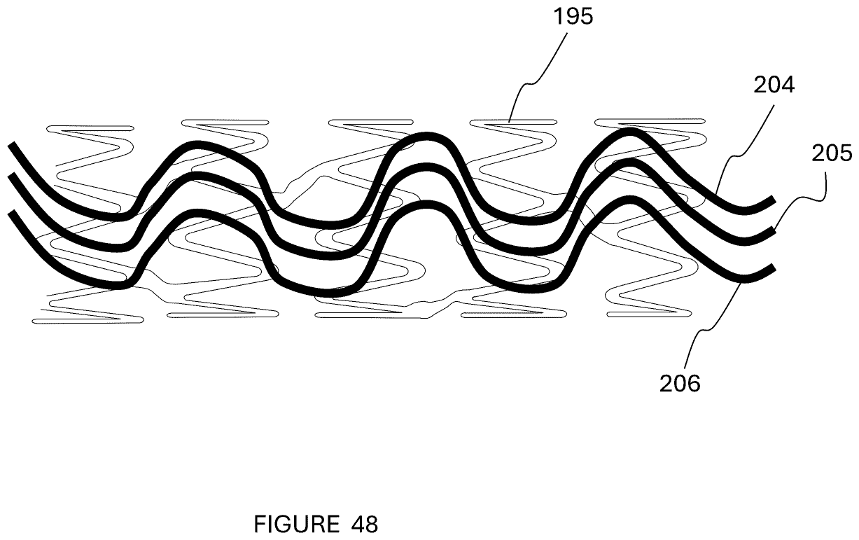

FIG. 47 shows another exemplary embodiment where the “amplitude” of the wire 204 is not as large as that of the wire 199 detailed above. In this regard, the wire takes up less space inside the inner diameter of the cage relative to that which is the case for wire 199 detailed above. There can be utilitarian value with respect to this arrangement by way of providing more room within the cage for other wires, such as is seen in FIG. 48, where there is wire 205 and wire 206 interleaved as seen with wire 204, which might not be as elegantly possible with the embodiment of FIG. 44.

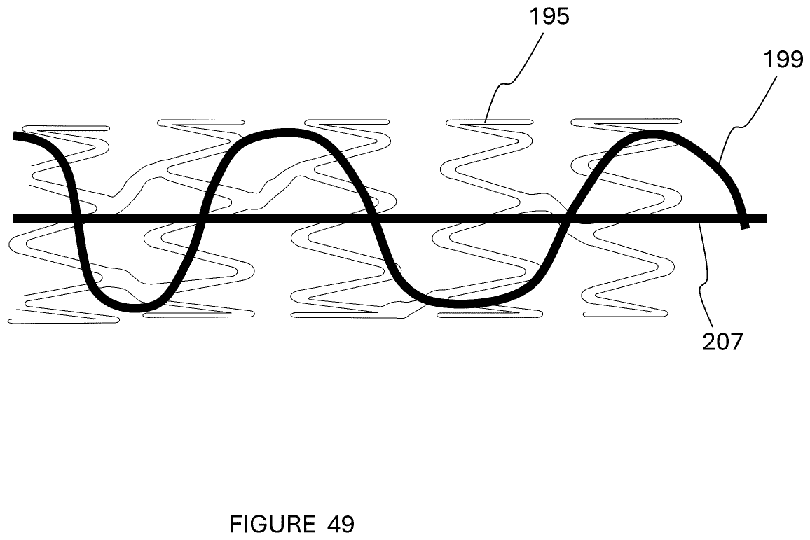

And while the embodiment of FIG. 48 is shown with the wires having the wavy (or serpentine) feature in the plane of FIG. 48, in other embodiments, wires can also be wavy (or serpentine) in different planes, such as a plane that is normal to that of FIG. 48, and the wires can be interleaved with each other albeit into and out of the plane in FIG. 48. This is seen in FIG. 49 by way of example, where wire 207 moves towards and away from the viewer of the figure to “weave” with wire 199. The planes on which the pattern extends can be located at any angle about the longitudinal axis of the elongate stimulating assembly, not just the right angle shown in the figures, and note that a given lead wire can have a pattern that follows different planes with different locations along the longitudinal axis as the lead wire extends along the elongate stimulating assembly.

In an embodiment, there are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55 or 60 or more or any value or range of values therebetween in 1 increment wires extending inside a given cage, and any one or more or all of the wires can be arranged according to any of the arrangements herein and any variation thereof that can have utilitarian value.

Note the wires could extend in and out of the lateral sides of the cage if such as utility. The wires could be woven with the cage.

Still with reference to FIG. 49, as can be seen, the “amplitude” of wire 199 is larger than those detailed above. In an exemplary embodiment, the “amplitude” of wired in 199 can extend the entire interior diameter of the cage 195. That said, in some embodiments, there is utilitarian value with respect to providing an offset between the wall of the cage and the lead wires, on one or both sides of the cage, which can, for example, reduce the likelihood of fraying or fretting. The arrangement of the wire(s) can be such that such will reduce a likelihood of damage to the wire insulation (so as to avoid short circuiting (such as shorting to the cage for example) or exposure to body fluid/tissue at locations where such is not desired), relative to that which would otherwise be the case, all other things being equal. In an embodiment, the amplitude of one or more or all of the wires is greater than, less than and/or equal to 100 (in which case there is no greater than), 90, 80, 70, 60, 50, 40, 30, 20 or 10% or any value or range of values therebetween in 1% increments of the local interior diameter of the cage, and note that the diameters need not be the same along the entire length of the cage and/or the length of one or more all the wires. That is, a given wire can have a spacing relative to the inner diameter of the cage that meets different values at different locations along the cage, providing that the art enable such.

Also, it is noted that the diameter D15 and/or the inner diameter of the cage can vary along the length of the cage. In an exemplary embodiment, various segments 202 can have different values of D12 and D15, etc.

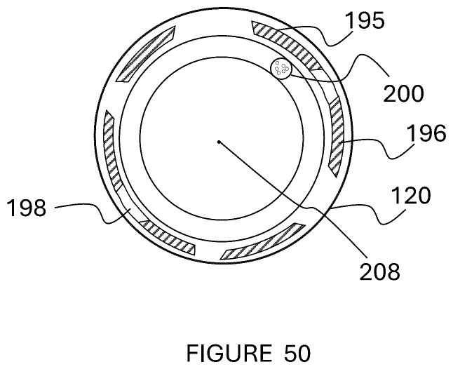

FIG. 50 presents a cross-sectional view of an exemplary stimulating assembly lying on a plane normal to the longitudinal axis thereof. This exemplary embodiment shows a cross-section of the cage 195 through a segment 202 of the cage, where the segment connectors 198, of which there are 2 in this exemplary embodiment, can be seen in the backdrop. In this exemplary embodiment, the cage 195 is embedded in a silicone body 120.

In this exemplary embodiment, there is a lead wire bundle 200, which includes a number of lead wires therein. In an exemplary embodiment, there can be any of the number of lead wires detailed herein and/or variations thereof, providing the art enable such. In this exemplary embodiment, there can be leads for each particular electrode array located in a tube that establishes the bundle. This can be done in a manner analogous to how Romex wiring is implemented. In this exemplary embodiment, the lead wire bundle 200 extends in a spiral manner about the longitudinal axis 208 of the stimulating assembly. That is, from a side view, the wire bundle 200 would extend in a manner analogous to a coil spring. In an exemplary embodiment, the wire bundle 200 can be embedded in silicone. In an embodiment, the wire bundle 200 is embedded in the silicone body 120. In another exemplary embodiment, the wire bundle is not embedded in the silicone body 120, but could be embedded in another silicone body inside the silicone body 120. In some embodiments, the wire bundle 200 is not embedded in any silicone. In an embodiment, there can be two or more wire bundles (offset from each other in any of the manners detailed herein or other ways that will enable the teachings herein).

In this exemplary embodiment, the wire bundle 200 extends entirely within the cage 195. And while the embodiment of FIG. 50 is presented in the context of a wire bundle where the lead wires are located inside the tube, in an alternate embodiment, the lead wires are not located in the tube, but the lead wires are still arranged in a spiral manner.

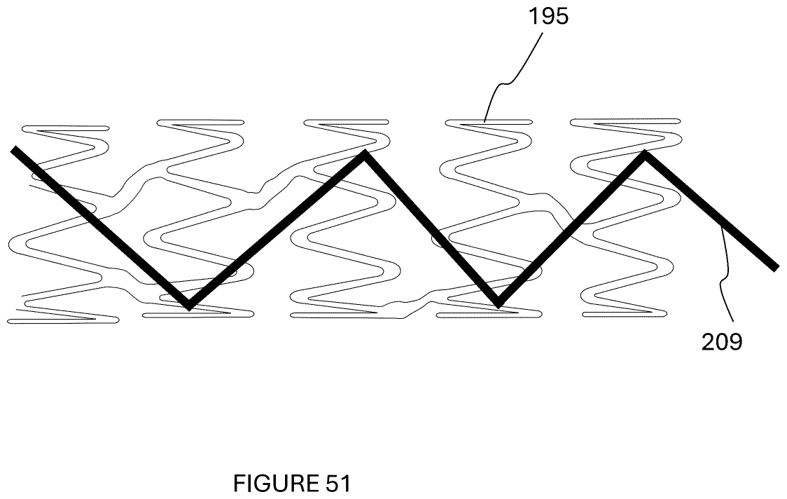

And arrangements of the lead wires in a spiral and a wavy (or serpentine) manner are not the only arrangements that are applicable to some embodiments. In an alternate embodiment, such as that seen in FIG. 51, the lead wires 209 can be arranged in a zigzag manner as shown. Different geometries can be utilized. In at least some exemplary embodiments, any geometry that can enable the teachings detailed herein and otherwise can have utilitarian value can be utilized to implement stimulating assemblies. Moreover, in an exemplary embodiment, any arrangement that can reduce stress and/or provide the ability of the lead wires to expand and/or contract globally or locally and/or avoid pinching or void the lead wires resisting the overall deformation of the stimulating assembly that is desired during the implantation process or otherwise ensuring in some embodiments that the lead wires do not provide spring back courses or at least reduce the likelihood that such results or at least reduce the overall force that results relative to that which would otherwise be the case can be utilized in at least some exemplary embodiments.

In view of the above, it can be seen that in some embodiments, there is an elongate stimulation assembly such as assembly 163 of FIG. 12 for example, or that of FIG. 16 for example, of an implantable stimulation device, such as cochlear implant 164 for example, that includes a first portion including a plurality of electrodes, such as electrodes 92. The assembly also includes lead wires, such as wires 121, extending from the first portion in electrical communication with the plurality of electrodes 92, the lead wires being located in an elongate lead body. Also, the assembly includes a cage component, such as cage 165, extending in an elongate manner at least partially along with the lead wires 121. In an embodiment, the lead wires extend within the cage component. In other embodiments, at least some of the lead wires extend outside the cage component. In an embodiment, the cage component is located on an outside of the assembly. In an embodiment, the cage component is located in an inside of the assembly (e.g., embedded in body 120, at least partially embedded in body 120). In an embodiment, the cage component is underneath silicone material. In an embodiment, the cage component is kink proofed. This can be based on the geometry/design of the cage by itself. This could also be implemented using an additional structure, such as, for example, a sheath inside the cage/a tube inside the cage, that resists bending beyond a certain point (owing to elastic deformation of the tube—the tube resists deformation of the cage beyond a certain amount and/or provides a radial force to the inside of the cage (analogous to pressurization of a pipe).

Concomitant with the teachings detailed herein that various embodiments can be combined with one another (embodiments include any one or more of the teachings detailed herein practiced in combination with any one or more of the other teachings detailed herein, providing that the art enable such, unless otherwise noted, in the interests of textual economy), in an exemplary embodiment, the elongate stimulation assembly can also include in addition to the cage component a malleable component that forms a helix.

Embodiments include the cage component that includes portions configured to more easily plastically deform than other portions of the cage component. In an exemplary embodiment, the cage component is configured so that a 1st force applied at one location in a given direction will plastically deforming a material, but that same force applied in the same direction but at another location will not plastically deform the material, and, in an exemplary embodiment, the force applied at the another location must be at least 1.1, 1.2, 1.3, 1.4, 1.5, 1.75, 2, 2.25, 2.5, 2.75, 3, 3.5, 4, 4.5 or 5 times any value or range of values therebetween in 0.1 increments that of the 1st force to achieve plastic deformation at the another location, all other things being equal.

In an exemplary embodiment, an extrapolated volume of the cage component is made up of less than 5, 10, 15, 20, 25, 30, 35, 40, 45, 50, 55, 60, 65 or 70% or any value or range of values therebetween in 1% increments of solid material. By extrapolated volume of the cage component, it is meant that the space between the stringers and the segment connectors in the joints, etc., would be considered part of the overall volume of the generally cylindrical structure, for example, of a cage structure. This is not the volume of a fluid that the cage would displace, but instead the volume of the fluid that would be displaced of the cage was a solid walled cylinder that was open on either end, for example. That is, this is the volume that would exist if the cage instead was a solid walled structure of a given thickness, where if the cage structure was machined from that solid wall structure (which would have a hollow interior—the machining would be normal to the contingent surface on the outside of the cylinder or otherwise the solid walled structure), that solid wall structure of the same length of the cage will correspond to the extrapolated volume. Put another way, with respect to a cage structure that extends parallel to the longitudinal axis of the cage structure at all locations, the extrapolated volume is the volume of a cylinder that has open ends having a wall thickness corresponding to the thickness of the stringers.

In view of the above, it can be seen that in an exemplary embodiment, there is a device, such as a cochlear implant for a retinal implants or a heart stimulation device, etc., that comprises by way of example only and not by way limitation, a stimulating assembly (e.g., assembly 163 as detailed above for example) of an implantable stimulating device (e.g., cochlear implant 164) including a lead assembly made at least partially of a structure having portions that are more susceptible to plastic deformation than other portions of the structure. In this exemplary embodiment, the device is configured to resist movement of at least a portion of the lead assembly, the movement of the lead assembly due to the plastic deformation. In an exemplary embodiment, the movement is spring back of the assembly. In an exemplary embodiment, the susceptibility to plastic deformation can be quantified in accordance with at least some of the teachings detailed herein with respect to forces applied at different locations. In an exemplary embodiment, the structure is configured to provide hinge points that undergo larger strains during bending of the structure than other portions adjacent the hinge points, which larger strains produced the plastic deformation. In an exemplary embodiment, the larger strains are at least 5, 10, 15, 20, 25, 30, 35, 40, 45, 50, 60, 70, 80, 90, 100, 125, 150, 175, 200, 225, 250, 275 or 300% in 1% increments than the other strains, all other things being equal.

Exemplary embodiment, the structure is a step structure. In this regard, it is noted that cage structures that can be implemented in at least some exemplary embodiments can correspond to structures that are utilized in stents to maintain an opening in a blood vessel or an artery or vein, etc. In this regard, by way of example only and not by way limitation, embodiments include utilizing commercially available stents structures in the stimulating assembly to enable the teachings detailed herein. That said, in an alternate embodiment, the cage structures correspond to specially designed structures for use in the elongate simulating assembly's to enable the teachings detailed herein.

In an exemplary embodiment, the structure is encapsulated in an overmoulded silicone body. In an embodiment, the body is not silicone, but is instead another type of material. The biocompatible material that can enable the teachings detailed herein can be utilized providing that the art enables such.

An exemplary embodiment, the structure has a first section extending along a longitudinal axis of the structure that extends about the longitudinal axis in a zig-zag manner with respect to axial direction. By axial direction, it is meant the longitudinal direction, and thus the “extends about” is the “orbital path of the cage about the longitudinal axis. For example, if one looks at the cage from the side, one sees zig-gags. In an embodiment, the structure extends in a serpentine manner with respect to the axial direction. In an embodiment, the structure extends in a wavy manner with respect to the axial direction.

In an embodiment, the structure has a first section extending along a longitudinal axis of the structure that extends about the longitudinal axis in an arcuate manner with respect to radial direction (what is seen when looking down the longitudinal axis). But this rases the point that in some embodiments, this is not the case. The cage could have an extrapolated square cross-section when viewed down the longitudinal axis. Thus, the structure would extend about the longitudinal axis in a digital manner with sections at right angles to each other. In this regard, the extension could be in a pentagon shape, a hexagon shape, an octagon shape, a triangle shape, etc., with the accompanying extension in the respective manner corresponding to those shapes.

In an embodiment, with respect to location along a longitudinal axis of the structure, the structure has at least 1, 2, 3, 4, 5, 6, 7, 8, 9, 10, 11, 12, 13, 14, 15, 16, 17, 18, 19, 20 or more or any value or range of values in 1 increments first cross-sections normal to the longitudinal axis (sections through the segment connector(s) 198) and at least at least 1, 2, 3, 4, 5, 6, 7, 8, 9, 10, 11, 12, 13, 14, 15, 16, 17, 18, 19, 20 or more or any value or range of values in 1 increments second cross-sections normal to the longitudinal axis (sections through the segments 202) respectively located in an alternating manner along the longitudinal axis and in some embodiments equidistant from each other with respect to location along the longitudinal axis and the first cross-sections have at least 10, 15, 20, 25, 30, 35, 40, 45, 50, 55, 60, 65, 70, 75, 80, 85, 90, 91, 92, 93, 94, 95, 96, 97 or 98% or any value or range of values therebetween in 0.1% increments less material of the structure than the second cross-sections by area. Note that the values need not be the same for given first and second cross-sections.

In an embodiment, the first cross-sections are less than, greater than and/or equal to 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 350, 400, 450, 500, 550×10⁻² mm or any value or range of values therebetween in 0.01 mm increments from the second cross-sections, and the values need not be the same for all the cross-sections.

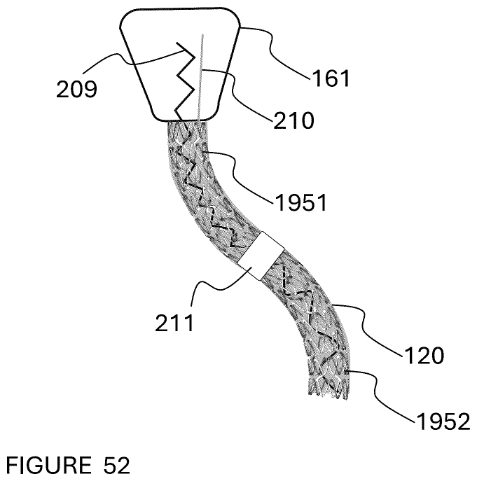

Embodiments include utilizing the cage for the conduction of electricity to one or more of the stimulating electrodes, and/or in an alternative embodiment, utilizing the cage as a return electrode or part of an electrical return system of the implantable medical device. FIG. 52 shows an exemplary embodiment of a portion of an elongate stimulating assembly according to such an exemplary embodiment. Here, the stimulating assembly includes a return band 211. This can be a thin walled metal cylinder by way of example, such as one made of platinum, that extends about the outer diameter of the body 120 by way of example. The return band 211 can be considered a large ring for example. The return band is electrically coupled to the cage structure that is embedded in the body 120. By way of example only and not by way of limitation, wires or some electrically conducting structure extends through the silicone body to the cage underneath the outer surface of the silicone body. The cage, which is made of an electrically conductive material, thus places the return band 211 into electrical conductivity with the electronics of the receiver stimulator. In this regard, the cage extends from the band 211 proximally towards the transition body 161. Shown in FIG. 52 is a wire 210 that is electrically coupled to the cage, which wire 210 electrically connects the cage to an ECE pin (for a cochlear implant-extra cochlear electrode (an electrode outside the cavity into which the “channel electrodes” or “stimulating electrodes” are located in an exemplary embodiment) of a feedthrough of the receiver stimulator by way of example. Also shown is lead 209, which is also connected to a pin of the feedthrough, but here, a pin for a stimulation channel of the receiver stimulator by way of example. Section 1951 is part of the cage that is “live.” Section 1952 is also part of the cage that is “live.” That is, electrical current flows through the cage, or otherwise can flow through the cage. Section 1951 is between, electrically speaking, band 211 and wire 210, whereas section 1952 is the opposite side, but it is still “live,” because it is electrically connected to the band 211 and the wire 210. Because the sections 1951 and 1952 are covered by the silicone body 120, these are electrically isolated from the environment of the stimulating assembly, other than for the fact that they are in electrical conductivity with the band 211. In an alternate embodiment, section 1952 can be part of that is completely separate or otherwise is not part of any part of the cage of which section 1951 is a part. Alternatively, section 1952 can be electrically isolated from band 211 and or cage 1951. Thus, section 1952 may not be a “live” component.

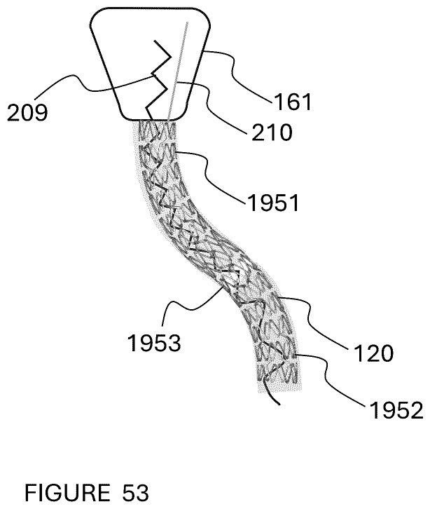

FIG. 53 shows another exemplary embodiment, where instead of band 210 or otherwise a separate component relative to the cage and in electrical conductivity therewith establishing the conductive component for the return that interfaces with/is exposed to the ambient environment of the stimulating assembly (electrically speaking), the cage material itself is used as the return body that interfaces with the ambient environment. Here, there is section 1953 of the cage which is exposed to the ambient environment, which can be a body fluid or to some tissue, etc., because there is no silicone body 120 at that section/no electrical insulation between the cage and the ambient environment (this “opening” can go completely around the longitudinal axis of the stimulating assembly, or around only part of the way-any amount of surface that can provide sufficient electrical interface with the ambient environment can be utilized). While the embodiments of FIGS. 52 and 53 show one return interface with the ambient environment, or at least one return section, other embodiments can include 2 or 3 or 4 or 5 or 6 or more return sections or any number that can have utilitarian value.

Also, while the embodiments of FIGS. 52 and 53 present a single cage, in other embodiments, 2 or more cages can be utilized, which cages could be electrically separated from each other. Thus, in an exemplary embodiment, there could be respective cages that have respective return interface sections, where the respective cages are electrically isolated from each other, and where, for example, there could be respective wires 210 placing the respective cages into electrical communication with the respective ECE pins of the feedthrough of the receiver stimulator. This respective wires could be extended in the interior of the cages in a manner that is the same as or analogous to the lead wires as detailed herein.

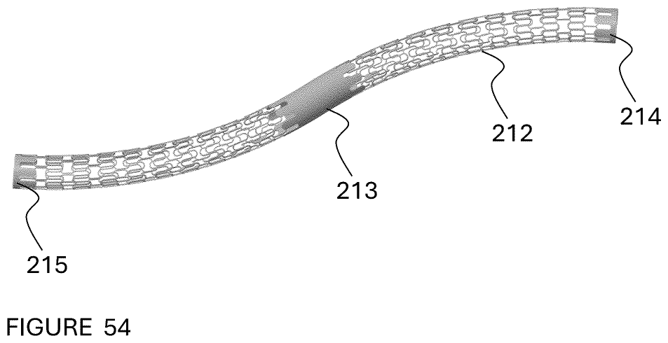

While the embodiment of FIG. 53 presents the return interface section as part of the cage, or more specifically, where the grid structure, such as that of a segment 202 of the cage, is utilize as the return interface, in other embodiments, the return interface can be a non-cage structure of the cage apparatus. By way of example only and not by way of limitation, FIG. 54 shows an exemplary cage apparatus that includes a cage section 212 and a non-cage section 213. Specifically, section 213 is generally in the form of a cylinder, where the cage 212, or more specifically, the grid structure of the cage 212 extends away therefrom. In an embodiment, this can be a monolithic structure (the entire component of FIG. 54) while in other embodiments, these can be separate components. With respect to the latter, cage 212 can be connected to the interface 213, such as by welding or a snap fit or an interference fit, etc. Also seen are distal and proximal end caps 214 and 215 of the cage apparatus of FIG. 54. As with the interface section 213, these can be monolithic with the cage structure 212, or can be connected thereto such as by welding or by a snap fit or interference fit, etc. No further that in an exemplary embodiment, elements 214 and/or 215 can also be utilized to interface with the ambient environment and establish a return structure. In this regard, this exemplary embodiment, depending on how the silicone body is applied and/or not applied or otherwise what is applied is removed, one or more or all of elements 213, 214 and 215 can be utilized to interface with the ambient environment. In an exemplary embodiment, the silicone body extends distally from element 213, and another silicone body extends laterally from element 213. These bodies could for example cover the end ¼^{ths} (longitudinal length—simply to choose a length for description purposes) of section 213 and extend away to the distal and proximal ends, so as to provide a single return interface section, or could extend to near the ends, to provide three return interface sections. Also, some portions of the cage structure 212 can be utilized to interface with the ambient environment. In an embodiment where all components are metallic and otherwise are in electrical communication with each other, there would be 3 return sections in this exemplary embodiment, for a single ECE “channel.” Said, in an exemplary embodiment, component 213 could be an electrically insulative material component, which holds the 2 different cage structures on either side thereof into a single assembly. Elements 214 and 215 could be the return structures that interface with the ambient environment. Respective separate wires could be attached to the respective cage structures, so as to electrically isolate the 2 structures from each other and thus provide 2 separate returns if such is utilitarian value. This could also provide redundancy in an exemplary embodiment.

In the interest of full disclosure, while the embodiments above for the most part have concentrated on the utilization of the cage structure or an assembly having a cage structure where the interface is utilized as a return interface, in other embodiments, the interface section that is exposed to the ambient environment can be utilized as a stimulating interface. Electrodes can be placed onto the cage structure or could be in electrical communication with the cage, and thus the cage can be used to provide electrical communication between the receiver-stimulator and the electrodes used to stimulate tissue.

In view the above, it can be seen that, in some embodiments, the exposed portion of the cage can be utilized as an electrode contact or return rather than a separate contact or return attached to the cage. But in other embodiments, separate electrode contacts and/or returns can be utilized where the cage provides electrical productivity between such and the receiver stimulator.

In view of the above, there is the stimulating assembly detailed wherein the assembly includes a plurality of stimulating electrodes (any of the numbers detailed herein by way of example). In an embodiment, the cage establishes an electrical return for a circuit that includes the assembly. This can be for the return electrode (extra-cochlear-electrode (ECE)). Concomitant with embodiments of implementing this feature detailed above, a portion of the cage component is covered with an electrically insulative material (e.g., the silicone of the body 120). Also, in some embodiments, a portion of the cage component is exposed to an ambient environment, thereby establishing the electrical return.

In an embodiment, the stimulating assembly includes a second cage component extending in an elongate manner partially along with the lead wires, wherein the lead wires extend within the second cage. In an embodiment, the cage component extends partially along with the lead wires and is spaced away from the second cage component in the longitudinal direction and the cage component is electrically isolated from the second cage component. Also, in an embodiment, the cage component establishes a first electrical return electrode and the second cage component establishes a second electrical return electrode.

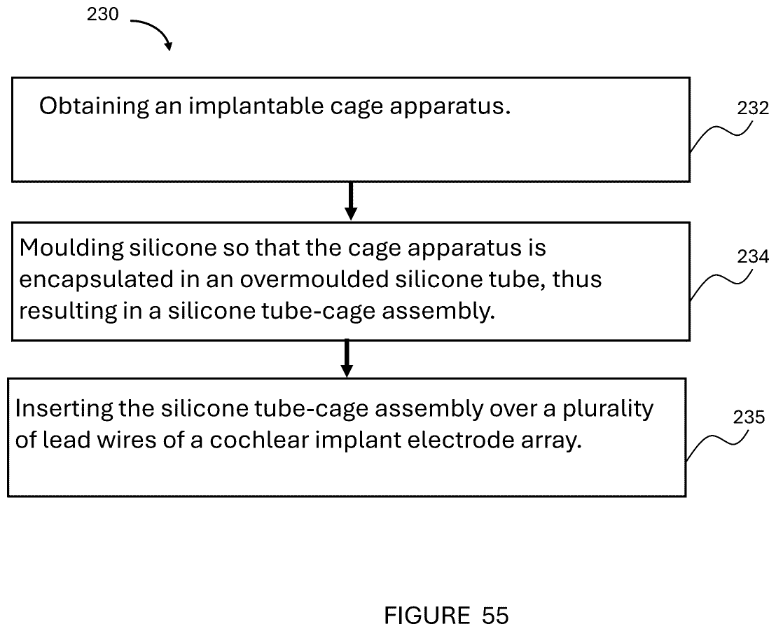

FIG. 55 provides an exemplary flowchart for an exemplary method, method 230. In an embodiment, the cage concepts detailed herein enable a manufacturing process that provides the ability to expand the cage in the radial direction. In an exemplary embodiment, for the purposes of conveying the concept, the cage could be encapsulated in an overmoulded silicone tube, expanded using a mandrel or a balloon, and inserted over the lead wires. In an exemplary embodiment, the cage and the over molded silicon tube is expanded using lenium). In an embodiment, the cage is then be plastically compressed down to a smaller diameter using an inflatable cuff, or a heat shrink operation, where the cuff is then cut away, or removed by some other action. Accordingly, method 230 includes method action 232, which includes the action of obtaining an implantable cage apparatus. This can correspond to obtaining any of the cage apparatus as detailed herein or variations thereof. This can include obtaining such from a subcontractor or supplier, or manufacturing the cage. Any action of obtaining the cage apparatus in whatever form that can be utilized to implement method 230 is covered by method action 232.

Method 230 also includes method action 234, which includes the action of moulding silicone so that the cage apparatus is encapsulated in an overmoulded silicone tube, thus resulting in a silicone tube-cage assembly. As can be executed by conventional techniques. Any device and/or system that can be utilized to and any method of achieving or otherwise implementing this method action, can be utilized in at least some exemplary embodiments providing that the art enables such unless otherwise noted.

Method 230 further includes method action 235, which includes the action of inserting the silicone tube-cage assembly over a plurality of lead wires of a cochlear implant electrode array and/or versa (inserting the leads into the assembly).

An exemplary embodiment, method 230 includes the action of expanding the silicone tube-cage assembly in a radial direction. In an exemplary embodiment, this action is executed prior to inserting the silicone tube-cage assembly over the lead wires/inserting the lead wires into the silicone tube-cage assembly, expanding the silicone tube-cage assembly in a radial direction. Consistent with the introduction of this concept presented above, in an exemplary embodiment, after inserting the silicon tube-cage assembly over the lead wires/inserting the lead wires into the silicone tube-cage assembly, there is the action of contracting the silicon tube-cage assembly in the radial direction. This can be done utilizing the above noted inflatable cuff, or utilizing a die and pressed system for example. Heat shrinking can be implemented. Any device and/or system that can be utilized to and any method of contracting the silicon tube-cage assembly can be utilized to implement the teachings detailed herein providing that the art enables such unless otherwise noted. In an exemplary embodiment, the action of contracting the silicon tube-cage assembly plastically deforms the silicon tube-cage assembly. In an exemplary embodiment, the plastic deformation can be located at the elbows/joints detailed above, with the resulting deformation corresponding to that which is detailed above or a variation thereof or something analogous thereto.

In an exemplary embodiment, the plurality of lead wires are arranged in a wavy and/or serpentine and/or zigzag lead configuration. Again, this arrangement can be in the manner detailed above or otherwise analogous to that detailed above. In an exemplary embodiment, the action of contracting the silicon tube-cage assembly does not result in the arrangement of the lead wires being changed although the specific geometry might be altered a bit. In this regard, if the lead wires are waived the prior to contraction, the lead wires will remain wavy after contraction, although the specific geometry of the weightiness might be different after contraction. The amplitude and/or phase may be different for example. But that does not change the overall configuration. This can be implemented utilizing any device and/or system that can enable such.

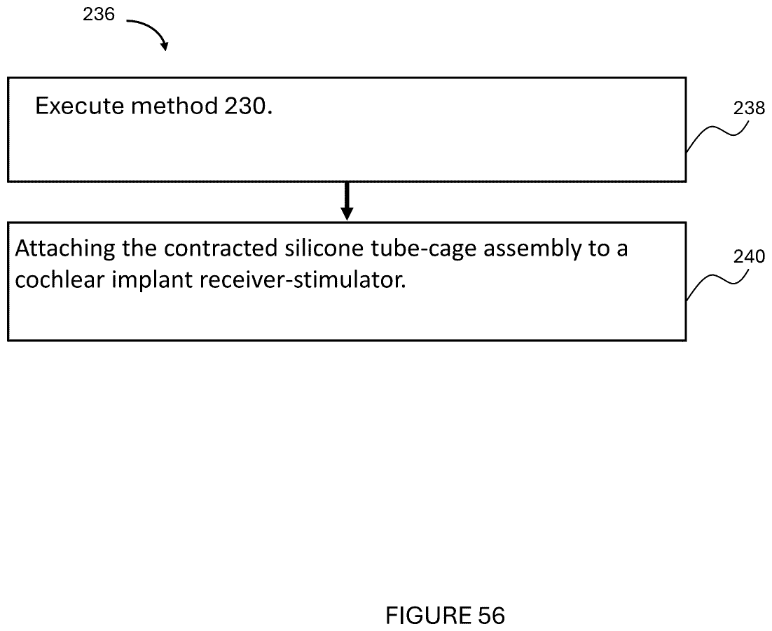

FIG. 56 presents another exemplary flowchart for an exemplary method, method 236, according to an exemplary embodiment. The method 236 includes method action 238, which entails executing method 230. Method 236 also includes method action 240, which includes the action of attaching the contracted silicon tube-cage assembly to a cochlear implant receiver-simulator. In an exemplary embodiment, this can include the action of attaching the lead wires to the pins of the feedthrough of the receiver stimulator. This can also include the action of establishing the transition body 161 detailed above, which can entail over molding silicone over the body 120 and the lead wires and potentially a portion of the silicon of the receiver-stimulator.

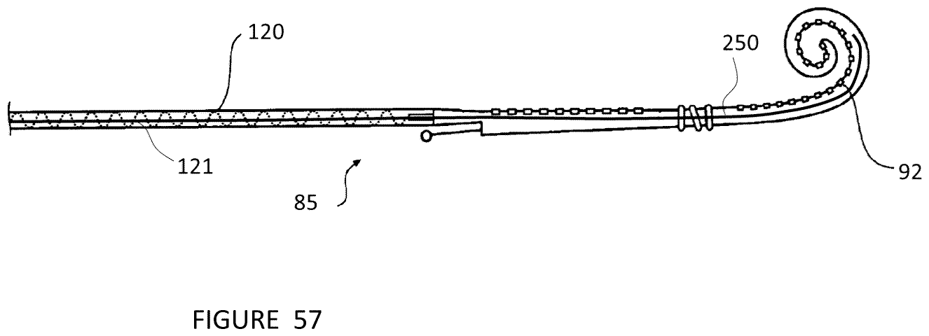

The teachings detailed above have generally been directed towards a structure that is located in the extra cochlear region/outside of the intracochlear region 117 of the elongate stimulating assembly. That is, all of the embodiments detailed above have disclosed the malleable portion/malleable structures being located in the lead assembly. Thus, the distal ends of any of the malleable portions or other structure detailed above to resist movement of a portion of the elongate stimulating assembly have all ended prior to reaching the intra-cochlea region. Indeed, most embodiments detailed above have been disclosed where the distal end of the malleable portion or other structure detailed above to resist movement of a portion of the elongate stimulating assembly of all ended prior to the end of the lead assembly 110 and have not extended into the electrical array 125. FIG. 57 depicts an alternate embodiment where the malleable portion (here, element 250) extends from the body 120 of the lead assembly into the electrode array 125 in general, and into the intra-cochlea region 117 of the electrode array in particular.

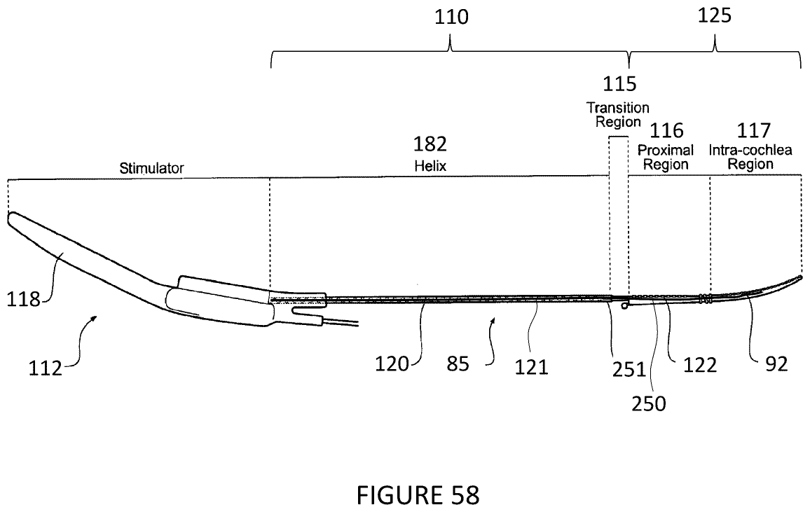

FIG. 58 corresponds to FIG. 2 above, except that malleable portion 250 is located inside the lead assembly and inside the electrode array. In an exemplary embodiment, malleable portion 250 corresponds to any of the malleable portions detailed above and/or to any of the structures detailed herein that have been disclosed to resist movement of a given portion of the elongate stimulating assembly due to elasticity or the like. FIG. 58 corresponds to FIG. 2 above, except that, as with FIG. 57, malleable portion 250 is located inside the lead assembly and inside the electrode array. As can be seen, malleable portion 250 extends all the way from the receiver stimulator 118 to the intracochlear region 117 of the electrode array 125. Thus, in an exemplary embodiment, malleable portion 250 is an elongated version of structure 149 of FIG. 11A above. That is, in FIG. 11A, the structure 149 extended at least substantially the full length between the receiver/stimulator 118 and the electrode array assembly 125. Here, structure 250 extends at least substantially the full length between the receiver/stimulator 118 and a location inside the intracochlear region 117 of the electrode array, such that the structure 250 is located in at least a portion of the intracochlear region 117 of the electrode array.

It is noted that in at least some exemplary embodiments, any of the aforementioned functionalities detailed above with respect to the malleable portions or otherwise structures detailed above that resist movement of the elongate stimulating assembly are applicable to the embodiments where the malleable portions/structures extend into the intracochlear region, both globally, and locally with respect to the portions of the elongate stimulating assembly that include the malleable portion/structure. By way of example only and not by way of limitation, to the extent that some of the embodiments resist movement at, for example, the midpoint of the lead assembly, owing to the fact that the malleable portion is located at the midpoint, the embodiments where the malleable portion or otherwise the structure is located within the intracochlear region also experience the phenomenon of resisting movement, at least with respect to the components that include the malleable portion/structure.

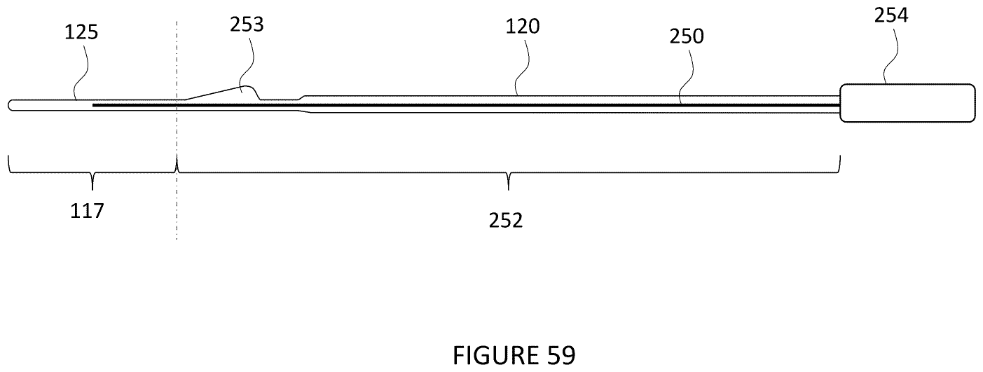

FIG. 59 depicts an exaggerated view of an implantable portion of a cochlear implant for purposes of discussion. The elongate stimulating assembly has been divided into two regions: the intracochlear region 117 and the extra cochlear region 252. As can be seen, the extra cochlear region 252 extends from the intracochlear region to the receiver stimulator 254, which corresponds to the receiver stimulator 118 detailed above. In this exemplary embodiment, structure 250 extends all the way from the receiver stimulator 254 to the electrode array, passing gripping nub/gripping wing 253 (which is utilized so as to provide the surgeon or other healthcare professional a body that can be gripped by a tool or the like during insertion, which body will not be damaged by the compressive forces applied thereto, and which body also provides a good reaction surface to react to torques and the like, along with other utilitarian value as is recognized in the industry) and into the intracochlear region 117. In this exemplary embodiment, the structure 250 is a monolithic structure that contiguously extends from the receiver stimulator 254 to the intracochlear region 117. Additional details of the structure will be described in greater detail below. However, it is briefly noted that any of the structures detailed above can be utilized with respect to the embodiments of FIGS. 57, 58 and 59, and any of the other embodiments detailed below unless otherwise specified.

Accordingly, in an exemplary embodiment, there is an elongate stimulation assembly of an implantable stimulation device, such as that depicted in FIG. 59 and the other figures detailed herein. In an exemplary embodiment, this assembly includes an intracochlear portion including an array of electrodes, such as by way of example only and not by way of limitation, intracochlear region 117 of electrode array 125. Still further, in an exemplary embodiment, this assembly includes an extra-cochlear portion extending from the intracochlear portion. With reference to FIG. 59, this is portion 252. Concomitant with the teachings detailed above, in an exemplary embodiment, the malleable portion, such as malleable portion 250, extends from the extra cochlear portion to the intracochlear portion such that the extra cochlear portion and the intracochlear portion also include respect to portions of the malleable component.

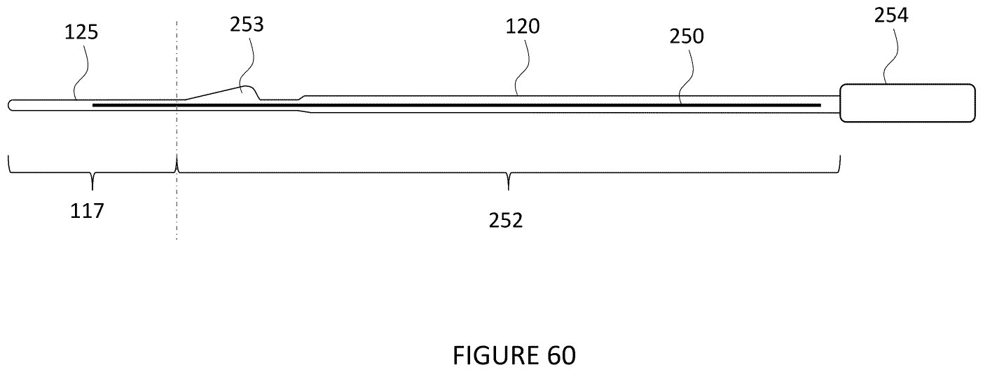

The embodiment of FIG. 59 depicts the malleable component 250 extending from the intracochlear portion 117 all the way to the receiver stimulator 254. However, in alternate embodiments, the malleable portion 250 does not extend all the way to the receiver/stimulator. FIG. 60 depicts such an exemplary embodiment, where the malleable portion 250 extends only to a location that is proximate to the receiver/stimulator 254. Thus, in an exemplary embodiment, in view of FIGS. 59 and 60, there is an elongate stimulation assembly that includes a malleable portion that extends at least to a location proximate to a receiver/stimulator.

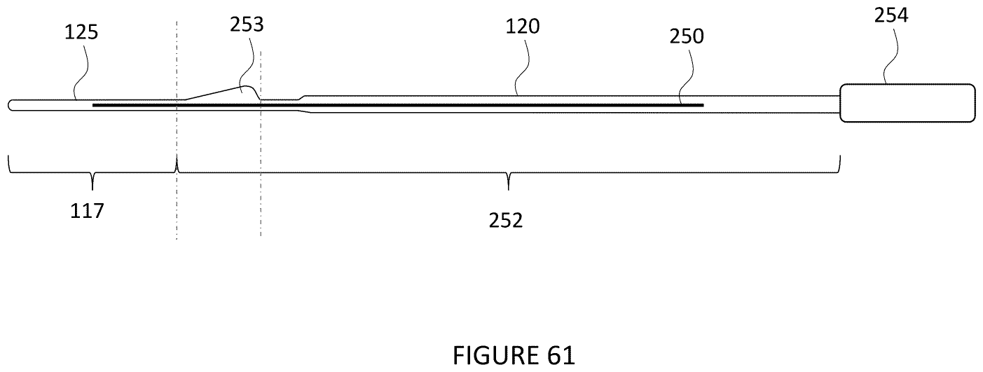

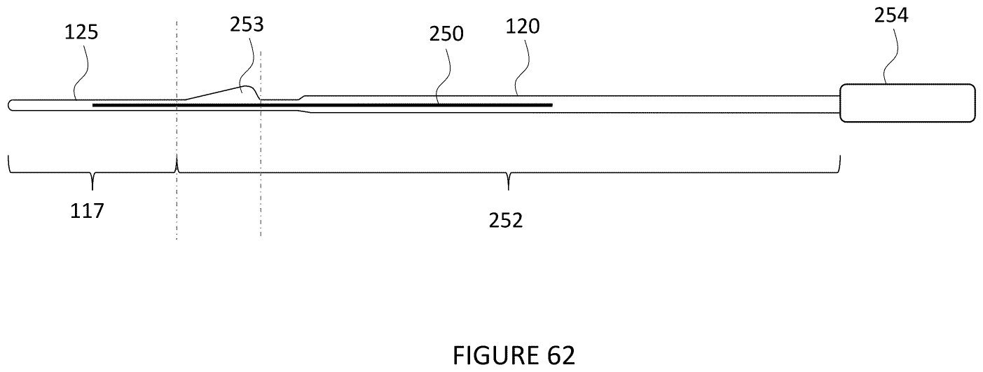

FIG. 61 depicts an alternate embodiment where the malleable portion 250 does not extend to a location proximate the receiver/stimulator 254. However, the malleable portion 250 extends over more than 50% the length of the lead assembly (the portion from the receiver/stimulator 254 to the beginning of nub 253, which in this embodiment begins the beginning of the lead assembly) and over more than 50% the length of the extra cochlear region 252 of the elongate stimulation assembly. In an exemplary embodiment, the malleable portion 250 extends more than 20%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% of the length of the lead assembly and/or the length of the intracochlear region, and/or extends a length of any value, or range of values therebetween in 0.1% increments the length of the lead assembly and/or the length of the intracochlear region (e.g., extends a length of 33.2% to 88.3% of the intracochlear region and/or the lead assembly, extends 55.5% the length of the lead assembly or the intracochlear region, etc.). In an exemplary embodiment, the malleable portion 250 extends the entire length. FIG. 62 depicts an alternate embodiment where the malleable portion 250 does not extend to a location proximate the receiver/stimulator 254. However, the malleable portion 250 extends less than that of FIG. 61 vis-à-vis the length of the lead assembly (the portion from the receiver/stimulator 254 to the beginning of nub 253, which in this embodiment begins the beginning of the lead assembly) and vis-a-vis the length of the extra cochlear region 252 of the elongate stimulation assembly. Note that in the interests of textual economy, disclosures correspond to the figures herein being shown to scale (and other embodiments correspond to disclosure where such is not the case). Thus, the arrangement of FIG. 62 can be considered to scale for purposes of conveying disclosure. Note that in an embodiment, any disclosure herein (any figure herein) can be relied on for scale purposes and what is scaled can be within 5%, 10%, 15%, 20%, 25%, 30%, 35%, 40%, 45%, 50%, 55%, 60%, 65%, 70%, 75% or 80% or any value or range of values therebetween in 1% increments of what is disclosed (the scaled values can be “adjusted” by any of those percentages).

In an exemplary embodiment, the malleable component extends from the intra-cochlear portion to a location at least proximate a housing containing a stimulator of the implantable stimulating device (e.g., the stimulator of the stimulator receiver 118), or the malleable component extends from the intra-cochlear portion to the housing.

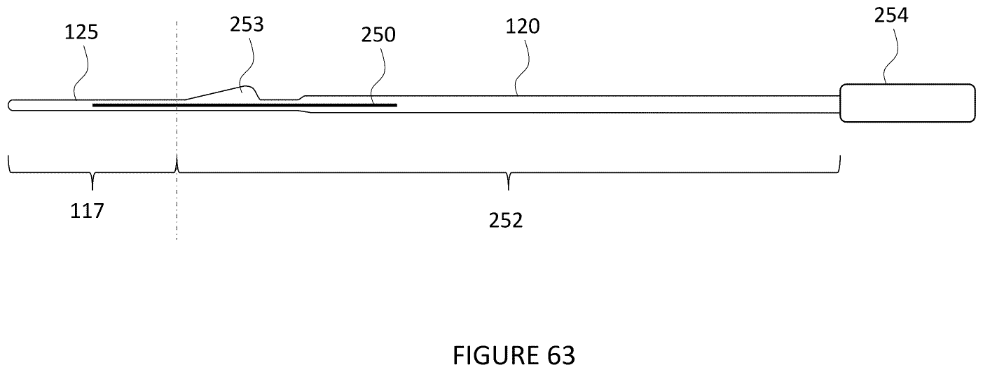

FIG. 63 depicts an exemplary embodiment where the malleable portion 250 extends a length that is less than 50% of the length of the lead assembly and less than 50% a length of the extra cochlear region 252. In an exemplary embodiment, the malleable portion 250 extends less than 10%, 15%, 20%, 25%, 30%, 33.333% (⅓^{rd}), 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% of the length of the lead assembly and/or the length of the intracochlear region. In an exemplary embodiment, portion 250 is a basil stiffener in that it provides stiffness to a basil portion of the intra-cochlear portion of the electrode array.

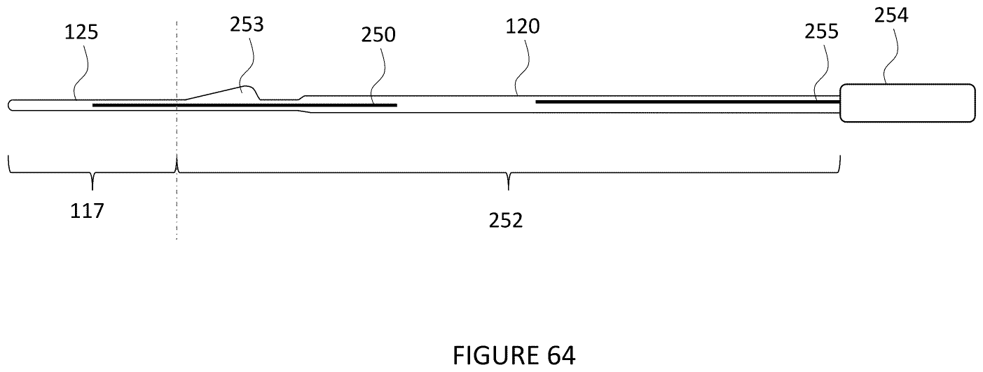

FIG. 64 depicts an alternate embodiment that includes two separate and distinct malleable portions: malleable portion 250 and the malleable portion 255. As can be seen, the malleable portions are separated by a space. Thus, in an exemplary embodiment, there is an elongate stimulation assembly according to the teachings detailed above, that further includes a second malleable component 255 that is separate from the malleable component 250 that extends to the intracochlear portion of the elongate stimulating assembly/that is located in the intracochlear portion. As can be seen, the second malleable component 255 is longitudinally spaced away from the malleable component 250 that extends to the intracochlear portion. By longitudinally spaced away from the malleable component, it is meant that there is a space in the longitudinal direction of the elongate stimulating assembly where neither the malleable portion 250 nor the malleable portion 255 are present. (It is noted that such an embodiment can include a third malleable portion located in such space, although in some other embodiments, it is noted that the space can be free of any malleable component.)

FIG. 64 also depicts that the malleable component 250 is laterally spaced away from the malleable component 255. That is, as can be seen, with the frame of reference of FIG. 64, malleable portion 255 is located “higher” than malleable portion 250. Note also that in some embodiments, not only are the malleable components not aligned in the vertical lateral component, in some other embodiments, the malleable components are not aligned in the lateral component extending in and out of the page that constitute to the frame of reference of FIG. 64. That said, in some alternate embodiments, the malleable portions are laterally aligned with one another in one dimension (any of the two) or laterally aligned in both dimensions. Note also that in some exemplary embodiments, the malleable portions about one another such that there is no space in the longitudinal direction between the two portions. Note also that in some exemplary embodiments, the two portions overlap in the longitudinal direction.

In an exemplary embodiment, the extra-cochlear portion and the intra-cochlear portion of the malleable portion, combined, extend a first length from a housing containing a stimulator of the implantable stimulation device (e.g., the housing of the receiver stimulator), and the second malleable component and the malleable component that extends to the intra-cochlear portion have a combined second length which is less than or equal to about 99%, 98%, 97%, 96%, 95%, 90%, 85%, 80%, 75%, 70%, 66.66% (⅔^{rds}), 65%, 60%, 55%, 50%, 45%, 40%, 35%, 33.333% (⅓^{rd}), 30%, 25%, 20%, 15%, or 10%, or any value or range of values therebetween in 0.1% increments, of the first length. In an exemplary embodiment, the portion of the malleable component that is a part of the intra-cochlear portion extends less than X % of a length of the intra-cochlear portion, and the portion of the malleable component that is part of the extra-cochlear portion extends less than Y % of the length of the extra-cochlear portion. In an exemplary embodiment, X and/or Y is 5%, 10%, 15%, 20%, 25%, 30%, 33.333% (⅓^{rd}), 35%, 40%, 45%, 50%, 55%, 60%, 65%, 66.667% (⅔rds), 70%, 75%, 80%, 85%, 90%, 95%, or 100% or any value or range of values therebetween in 0.1% increments.

It is noted that at least some of the embodiments detailed above have been disclosed in terms of the placement of the malleable portion at a location offset from the longitudinal axis of the elongate stimulating assembly, and/or have been disclosed in terms of placement of the malleable portion relative to the electrical leads of the lead assembly. In some exemplary embodiments, the embodiments that utilize the intracochlear malleable portion also utilize such features as disclosed above. Accordingly, in an exemplary embodiment, any of the teachings detailed above with respect to the extra cochlear malleable portion/structure that limit or otherwise prevent movement are also applicable to the embodiments directed to the intracochlear malleable portion/structure. That said, in some alternate embodiments the teachings relating to the placement of the malleable portion at a location offset from the longitudinal axis and/or placement of the malleable portion relative to the electrical leads are not applied to the embodiments that utilize the intracochlear malleable portion and/or structures that resist movement owing to the elastic properties of the elongate stimulating assembly. That is, in an exemplary embodiment, the intracochlear malleable portion is located at the longitudinal centerline of the elongate stimulating assembly. That is, in an exemplary embodiment, there is an elongate stimulation assembly of an implantable stimulation device, wherein a portion thereof includes a malleable component as detailed herein and/or variations thereof that extends from the intracochlear portion to a location in the extra cochlear portion, the extra-cochlear portion includes a plurality of electrical lead wires in electrical communication with the array of electrodes. There is also a malleable component extending in an elongate manner such that at least a portion of the malleable component is located closer to a longitudinal axis of the extra-cochlear portion than a portion of least one of the electrical leads of the plurality of electrical leads. Note also that this could be the case with respect to the malleable portion that is entirely located external to the intracochlear portion, at least when utilized with embodiments that include a separate malleable portion located in the intracochlear portion.

Also, in an exemplary embodiment, there is an elongate stimulation assembly of a cochlear implant, comprising an intra-cochlear portion including an array of electrodes, lead wires extending from the intra-cochlear region in electrical communication with the array of electrodes, the lead wires being located in an elongate lead body, and a malleable component extending in an elongate manner at least partially along with the lead wires, wherein the malleable component is located further from an outer surface of the lead body than at least one of the lead wires or wherein the malleable component is located the same distance from the outer surface of the lead body as at least one of the lead wires, wherein the malleable component has a portion extending into an intra-cochlear region. Also, in an exemplary embodiment, these aspects are also the case with respect to the second or third malleable component that does not have a portion in the intra-cochlear region but is used in conjunction with another malleable portion in the intra-cochlear region (e.g., 255 when used with 250 as seen in FIG. 64, etc.). In any event, any of the teachings detailed herein with respect to one embodiment can be combined with respect to another embodiment unless otherwise specified, providing that the art enables such combination.

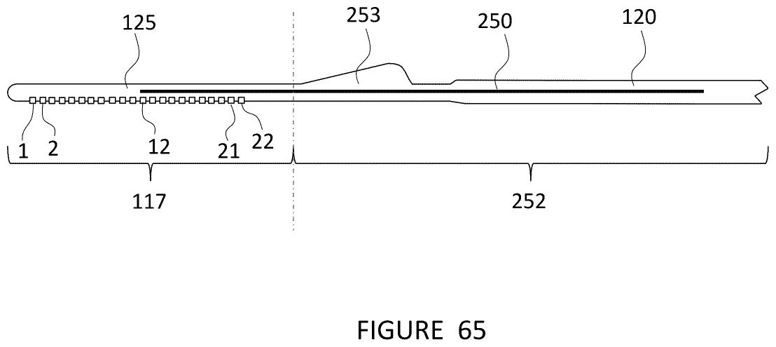

FIG. 65 depicts some additional details of the exemplary intracochlear malleable portion 250. Specifically, FIG. 65 depicts a close-up view of the electrode array 125 and a distal portion of the lead assembly in general, and a distal portion of the body 120 of the lead assembly in particular. As can be seen, malleable portion 250 extends only a portion of the length of the array of electrodes. In the embodiment of FIG. 65, there are 22 separate electrodes, and these electrodes are numbered from 1 to 22. It is noted that in at least some other embodiments, fewer electrodes or more electrodes will be utilized. In an exemplary embodiment, the electrode array has 2, 3, 4, 5, 6, 7, 8, 9, 10, 11, 12, 13, 14, 15, 16, 17, 18, 19, 20, 21, 22, 23, 24, 25, 26, 27, 28, 29, or 30, or more electrodes. In any event, the below teachings will be described in terms of the 22 electrode array.

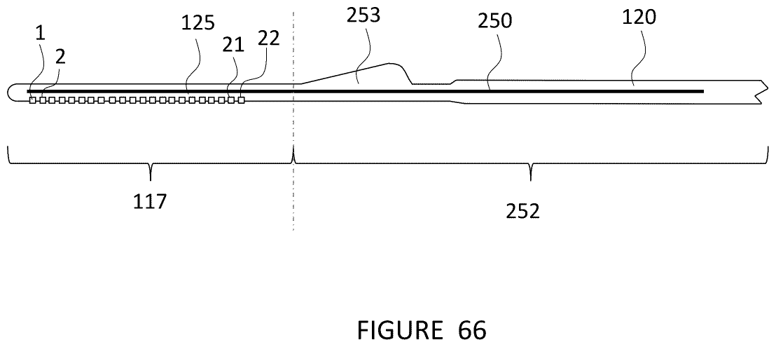

As can be seen, the malleable portion 250 extends only past electrode numbers 12-22 of electrodes 1-22. Thus, in an exemplary embodiment, the malleable component of the electrode array extends past more than 50% of the electrodes of the electrode array. That said, in some embodiments, the malleable component of the electrode array extends past fewer than 50% of the electrode array, while extending past at least one electrode of the electrode array. Conversely, FIG. 66 depicts an exemplary embodiment where the malleable component 250 extends past all of the electrodes of the electrode array (here, all 22 electrodes).

In an exemplary embodiment, the malleable component does not extend past more than 99%, 98%, 97%, 96%, 95%, 90%, 85%, 80%, 75%, 70%, 66.66% (⅔rds), 65%, 60%, 55%, 50%, 45%, 40%, 35%, 33.333% (⅓^{rd}), 30%, 25%, 20%, 15%, or 10% of the total number of electrodes of the electrode array, or any value or range of values therebetween in 0.1% increments. Accordingly, in an exemplary embodiment, the malleable component does not extend past with two thirds of the electrodes of the electrode array. In an exemplary embodiment, the malleable component extends past more than 99%, 98%, 97%, 96%, 95%, 90%, 85%, 80%, 75%, 70%, 66.66% (⅔rds), 65%, 60%, 55%, 50%, 45%, 40%, 35%, 33.333% (⅓^{rd}), 30%, 25%, 20%, 15%, or 10% of the total number of electrodes of the electrode array, or any value or range of values therebetween in 0.1% increments. Accordingly, in an exemplary embodiment, the malleable component extends past all of the electrodes or past 90% of the electrodes.

In an exemplary embodiment, the array of electrodes includes at least XX electrodes arrayed along a longitudinal direction of the intra-cochlear portion; and the malleable component extends from the extra-cochlear portion into the intra-cochlear portion such that the malleable component extends past at least YY of the electrodes of the electrode array. In an exemplary embodiment, XX is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 or more and YY is any of XX providing that it is not larger than a given XX, and zero. In an exemplary embodiment, XX can be 10 and YY can be 0, 1, 2, 3, 4, 5, 6, 7, 8, 9, or 10 (but not 11 or more, as that would exceed 10). In an exemplary embodiment, XX can be 22, and YY can be any number from and including 0 to and including 22.

It is noted that at least some exemplary embodiments include a single malleable component that extends at least substantially the entire length of the extra cochlear portion at substantially the entire length of the intracochlear portion. In an exemplary embodiment, the malleable component extends at least substantially the entire length of the extra cochlear portion and no more than about 80% of the intracochlear portion.

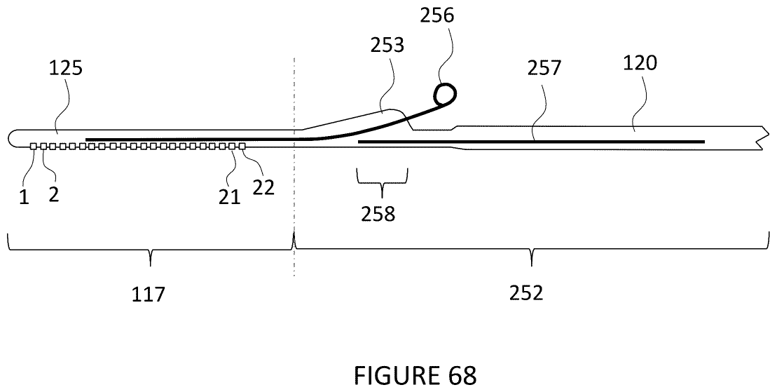

The figures detailed herein that include the electrodes are, in some embodiments, representative of electrode arrays where the distance from the tip of the electrode array to the most proximal electrode (e.g., electrode 22 in FIG. 68), is 5, 6, 7, 8, 9, 11, 12, 13, 14, 15, 16, 17, 18, 19, 20, 21, 22, 23, 24, 25, 26, 27, 28, 29, or 30 mm plus or minus 1 mm for any of the aforementioned dimensions, or any value or range of values therebetween in 0.1 mm increments (e.g., 20-25.3 mm, 8.1 to 10.5 mm, etc.).

It is noted that in some embodiments, element 250 is not a malleable component, but instead a component that is not malleable. For example, element 250 can be instead a Nitinol component or a component that is elastic, including super elastic. (Some additional details of this are described below.) Accordingly, in an exemplary embodiment, there is an elongate stimulation assembly of an implantable stimulation device, comprising an intra-cochlear portion including an array of electrodes, and an extra-cochlear portion extending from the intra-cochlear portion, wherein a first malleable component (e.g., element 255) is located in the extra-cochlear portion, and a stiffener component (e.g., element 250) is located in the intra-cochlear portion, the stiffener component being separate from the first malleable component. To be clear, in an exemplary embodiment, the stiffener can be a malleable component. Accordingly, in an exemplary embodiment, the stiffener component is a second malleable component separate from the first malleable component.

Concomitant with various embodiments described above, irrespective of the makeup of the stiffener component, the stiffener component has a first portion located in the intracochlear portion and a second portion located in the extra cochlear portion. In this regard, the relative dimensions detailed above with respect to the malleable portion that has component is located in the intracochlear portion the extra cochlear portion are also applicable to the stiffener of these embodiments.

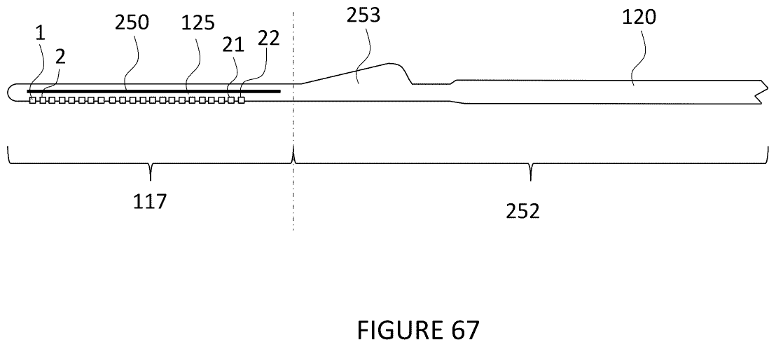

FIG. 67 depicts an alternate embodiment where the stiffener 250 is located only in the intracochlear portion 117. That is, in an exemplary embodiment, the stiffener does not extend into the extra cochlear portion. Corollary to this is that in at least some exemplary embodiments, the first malleable component does not extend into the intracochlear portion.

FIG. 68 depicts another exemplary embodiment where the stiffener 256 is in the form of a removable stylet. Some additional details of the stylet 256 will be described below. However, it is noted that stylets of the removable type enable a surgeon or other healthcare professional to retract or otherwise remove the stylet from the electrode array as the electrode array is being inserted into the cochlea so as to maintain a relatively stiffer portion at the local portion of the electrode array where the stylet is still present, while permitting the stylet to flex or otherwise bend by reducing the stiffness of the portion at the local portion of the electrode array from which the stylet has been removed.

In some exemplary embodiments, the stylet or any other removable stiffeners detailed herein and/or variations thereof can have a stopper to prevent overinsertion of the stylet into the elongate stimulating assembly. This stopper can be a moulded ring of silicone that extends about the stylet and/or can be a larger diameter handle from which the stylet protrudes, to facilitate handling. In an exemplary embodiment, the stopper can be a portion of the stylet that curves backwards towards the distal end of the stylet, so as to abut the interfacing surface of the elongate stimulating assembly.

It is noted that the removable stiffeners detailed herein can be a fixed single tool that is sterilizeable/resterilizeable, for multiple uses, or can be single use items.

In the embodiment of FIG. 68, the first malleable component is element 257. Element 257 is a totally extra cochlear component. As can be seen, the first stiffening component 256 overlaps in the longitudinal direction of the elongate stimulation assembly with the first malleable component 257. That said, in an alternate embodiment, there is no overlap in the longitudinal direction. Also, while the embodiment depicted in FIG. 68 is such that the first malleable component 257 does not extend into the intracochlear portion 117, in an alternate embodiment, the first malleable component 257 extends into the intracochlear portion 117. While the embodiments detailed herein with respect to element 256 have been directed to a removable stylet, the teachings detailed herein with respect to element 256 can also be applicable to a component that is not removable (e.g., element 250 as applied in FIG. 67). Again, any feature of any embodiment disclosed herein can be combined with any other feature of any other embodiment disclosed herein unless otherwise noted. In this vein, while the embodiment of FIG. 68 has been described in terms of a single completely extra cochlear malleable component (element 257), in an exemplary embodiment, two or more completely extra cochlear malleable components can be utilized with the stiffener. Accordingly, in an exemplary embodiment, there is an elongate stimulating assembly including a first and a second malleable component that are separate from one another, and are completely located in an extra cochlear portion of the elongate stimulating assembly, used in combination with a stiffener that includes at least a portion that is located in an intracochlear region of the elongate stimulating assembly, which stiffener is separate from the first malleable component and the second malleable component. In an exemplary embodiment, there can be a third malleable component that is completely located in an extra cochlear region. A fourth and/or a fifth and/or a sixth or more malleable components can be provided in the extra cochlear region. In at least some exemplary embodiments, one or more or all of the malleable components having portions located in the extra cochlear region or longitudinally spaced away from one or more or all of the other malleable components and/or the stiffener, while in other embodiments, one or more or all of the malleable components overlap one another in the longitudinal direction and/or one or more of the malleable components overlaps with the stiffener in the longitudinal direction.

Note also that in at least some exemplary embodiments, one or more of the stiffener components can extend into the intracochlear region from the extra cochlear region. In a similar vein, the stiffener component that has a portion in the intracochlear region can be completely located in the intracochlear region, while in other embodiments the stiffener can have a portion that is located in the intracochlear region while including another portion that is located in the extra cochlear region. Also, in an exemplary embodiment, one or more of the malleable components that are located in the extra cochlear region can be the only malleable component that is completely outside the intracochlear region of the elongate stimulating assembly. Corollary to this is that in at least some exemplary embodiments, the stiffener is the only malleable component that includes a portion that is located in the intracochlear region.

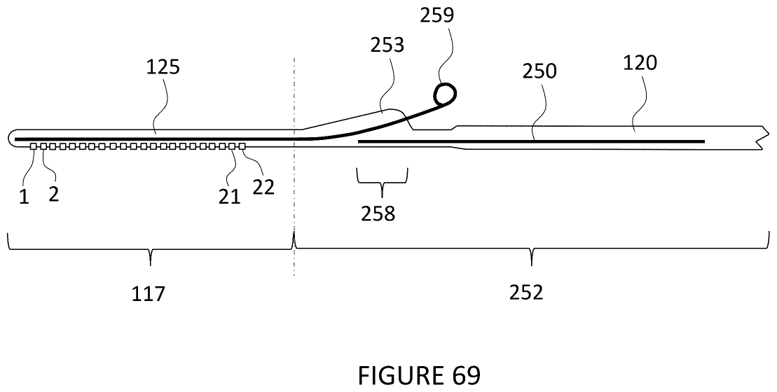

It is noted that the aforementioned lengths of extension of the malleable component that is located in the intracochlear region are also applicable to the embodiments that utilize a stiffener that is located in the intracochlear region, whether that stiffener is removable or not removable. Accordingly, FIGS. 68 and 69 depict some exemplary lengths of extension of the stylet, where stylet 259 of the embodiment of FIG. 68 extends past all but the most distal six (6) electrodes, while stylet 259 of the embodiment of FIG. 69 extends past all of the electrodes of the electrode array. Indeed, as can be seen, the stylet 259, prior to removal, extends all the way to a location proximate the tip of the electrode array 125 (effectively extending to the tip—the embodiment of FIG. 69 maintains a portion made up of the material that is utilized to carry the electrodes (e.g., silicone) or other material that is located between the tip of the electrode array and the tip of the stylet, as there is utilitarian value with respect to not having the tip of the stylet extend beyond or be flush with the tip of the electrode array. In this regard, at least some exemplary embodiments have a portion located between the tip of the electrode array and the tip of a stylet such that the portion will prevent the electrode array from being pushed in the proximal direction relative to the stylet 259. That is, the structural integrity of the distal portion of the electrode array 125 at least in the area between the tip of the stylet and the tip of the electrode array is such that longitudinal forces applied to the electrode array having a vector in the proximal direction likely to be experienced during insertion of the electrode array into the cochlea with the stylet 259 fully inserted therein (due to friction on the outside diameter of the electrode array as the electrode array interfaces with the cochleostomy, due to the tip of the electrode array contacting bone or the like, etc., all while an insertion force applied to the electrode array (typically via a tool gripping the nub 253) have a vector in the distal direction) will not drive the carrier portion of the electrode array 125 “downward” such that the stylet 259 pierces or otherwise breaks the tip and extends through the tip. In an exemplary embodiment, a boot can be located in the electrode array that provides a barrier between the tip of the electrode array and the tip of a stylet to prevent such protrusion. The point is, by “the removable stiffener extending to a location proximate a tip of the intracochlear portion” of the electrode array, it is meant that there is a portion of the electrode array that is positioned between the tip of the stylet and the tip of the electrode array that is present for at least structural reasons.

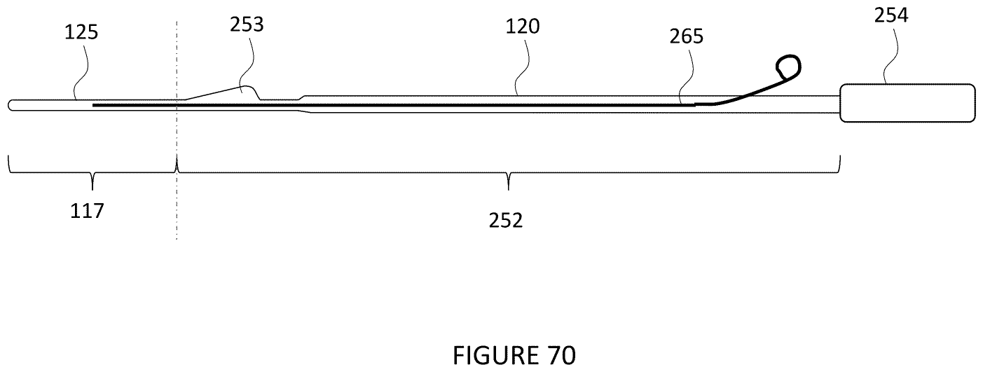

Also that the removable stiffener concept is not limited to simply utilization thereof in the intracochlear regions of the elongate stimulating assembly. The removable stiffener can be located in the extra cochlear portions as well, or, more accurately, a substantial portion of the elongate stiffener can be located in the extra cochlear portions. To this end, FIG. 70 depicts a stiffener 265 that is removable that extends a substantial length of the lead assembly/extends a substantial length of the extra cochlear region 252 of the elongate stimulating assembly. That said, in some alternate embodiments, the stiffener 265 extends less than a substantial length of the lead assembly/extra cochlear region 252. In an exemplary embodiment, any of the aforementioned extension lengths of the malleable components detailed above are applicable to the stiffener 265 or any of the other stiffener detailed herein removable or the like. In an exemplary embodiment, the stiffener 265 is malleable, while in other embodiments, the stiffener is elastic. In an exemplary embodiment, the stiffener 265 can be removed, or more accurately, retracted, a distance such that the stiffener 265 is not located in the intra-cochlear portion 117 of the electrode array, but still remains in the extra cochlear portion, such as by way of example only and not by way limitation, such that the tip is located approximately of the beginning of the lead assembly/distally of the end of the electrode array assembly. Thus, in an exemplary embodiment, the stiffener 265 can be utilized as a stylet during insertion of the electrode array into the cochlea, which stylet is removed from the cochlea after insertion of the electrode array, but can also be utilized as a stiffening member for the lead assembly. In an exemplary embodiment, the surgeon could clip or otherwise separate the portion of the stiffener that extends out of the lead assembly at a location at or below the surface of the lead assembly so that the portion of the stiffener that has been retracted from the elongate stimulating assembly does not irritate or otherwise cause problems after implantation. In an exemplary embodiment, the surgeon or the like can put a piece of material or the like over the opening so as to ensure that the stiffener will not migrate further out of the assembly. Alternatively, and/or in addition to this, this material can also be utilized to make sure that the stiffener does not migrate forward into the electrode array after implantation. In an exemplary embodiment, the electrode array assembly can be configured to be crimped or otherwise to be manipulated so as to prevent movement of the stiffener, or at least the portion of the stiffener that remains in the elongate stimulating assembly.

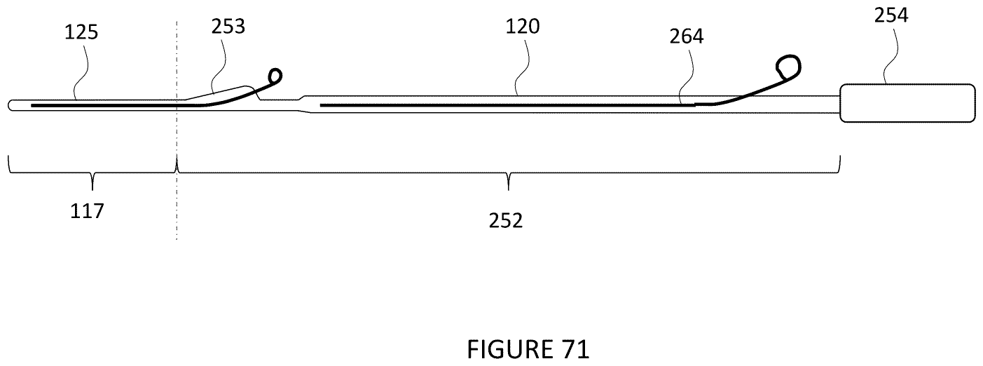

While the embodiment of FIG. 70 depicts the stiffener 265 extending from the extra cochlear region 3180 into the intracochlear region, in an alternate embodiment, there are a plurality of removable stiffeners. FIG. 71 depicts such an exemplary embodiment, wherein the intracochlear stiffener is located in the electrode array, and a completely extra cochlear removable stiffener 264 is located in the extra cochlear portion.

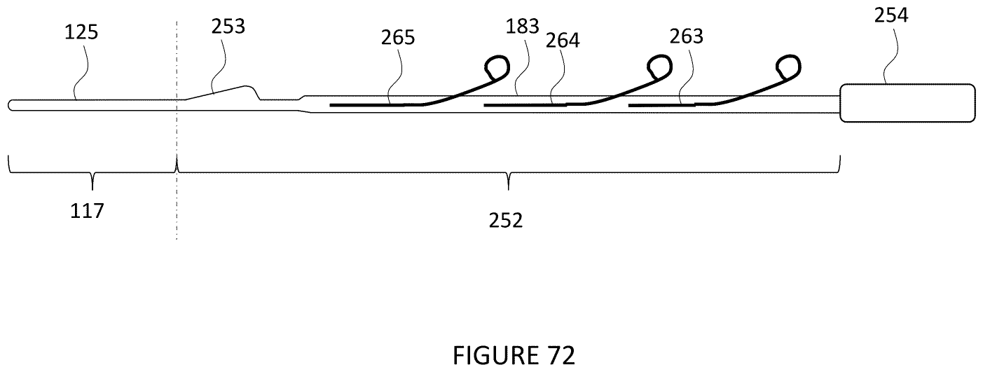

Note also that the removable features of the stiffener can have utilitarian value with respect to achieving control of the local relative stiffness of the electrode array. In this regard, a plurality of removable stiffeners can be located in the extra cochlear region. FIG. 72 conceptually depicts an exemplary embodiment that includes a first removable stiffener 263, a second removable stiffener 264, and a third removable stiffener 265, each laterally spaced apart from one another along the lead assembly. In an exemplary embodiment, a surgeon or other healthcare professional can selectively remove one or more of these stiffeners so as to control or otherwise establish a local stiffness of the lead assembly. For example, an exemplary scenario can entail a surgical procedure where the surgeon finds that the elasticity of the lead assembly must be combatted at generally the midpoint of the extra cochlear portion 252, but not at other portions away from the midportion. Thus, the surgeon can remove stiffener 265 and stiffener 263, while maintaining 264 in place. Alternatively, the surgeon or other healthcare professional may find that stiffness has utilitarian value at locations everywhere save for the location closest to the receiver stimulator 254. Thus, the surgeon or other healthcare professional will remove the stiffener 263, while maintaining the others in place. It is noted that in at least some exemplary embodiments, the elongate stimulation assembly can be provided with features so as to maintain the removable stiffeners in place if the surgeon chooses not to remove such. This can provide for prevention of migration or the like of the removable stiffener from the elongate stimulation assembly after implanted into the recipient.

While the embodiment depicted in FIG. 72 depicts at least the portions of the stiffeners that are inside the lead assembly as being longitudinally spaced from one another, thus leaving a location where the lead assembly is not stiffened beyond that which results from the general structure thereof, in an alternate embodiment, the stiffeners can extend past one another, or be located such that ends thereof are located, with respect to the longitudinal direction, where the beginnings of others begin, at least until removed.

In some exemplary embodiments, the handle portions of the removable stiffeners can be removed by the surgeon after implantation and/or prior to implantation if the surgeon deems that the removable stiffener should not be removed (i.e., the removable stiffener should be in place after implantation of the implantable component).

In a similar vein to the embodiment of FIG. 72, in an exemplary embodiment, the elongate stimulation assembly can be configured with a plurality of openings along the length thereof (e.g., spaced every 10 mm, every 15 mm, spaced in an uneven manner, etc.) that leads to an inner channel of the lead assembly. In an exemplary embodiment, the surgeon or other healthcare professional determines where added stiffening has utilitarian value, and inserts the stiffener into the lead assembly at those locations such that the inserted stiffener extends into the channel and along the longitudinal length of the lead assembly a desired length. In this regard, in an exemplary embodiment, a kit can be provided with a plurality of different stiffeners having different lengths (duplicate stiffeners can also be provided), and the surgeon or other healthcare professional can insert the stiffener of a desired length at a desired location to achieve a local desired stiffness. This can be done a plurality of times. Indeed, configurations can be provided so that the stiffeners can overlap one another such that one local region of the lead assembly can be stiffer than another region of the lead assembly that also has the stiffeners. Note also that in an exemplary embodiment, the surgeon can shorten the stiffener to his or her liking. That is, one or more stiffeners of a long length (e.g., longer than the length of the elongate stimulating assembly) can be provided to the surgeon or other healthcare professional, and the surgeon or other healthcare professional can shorten the lengths of the stiffeners as is found to be utilitarian, and then insert the shortened stiffeners into the elongate stimulating assembly.

Corollary to the above is that in an alternate embodiment, one or more stiffeners can be provided in the array assembly, which stiffeners can be frangible or the like at certain locations upon the application of a pressure and/or upon the application of some other stimulus (e.g., ultraviolet radiation, etc.). The surgeon can break or otherwise weaken the stiffener at certain locations to reduce the stiffness of the elongate stimulation assembly at those locations where such has utilitarian value.

Accordingly, in an exemplary embodiment, there is an elongate stimulating assembly including an extra cochlear portion, wherein the extra cochlear portion is configured to have a stiffness that is adjustable at a local portion thereof. In an exemplary embodiment, the adjustability results from the ability to remove and/or insert a stiffening component as detailed above and/or as would otherwise be enabled by the art. In an exemplary embodiment, adjustability results from the configuration where the malleable component located in the extra cochlear region has a stiffness that is adjustable at a local portion thereof (e.g., due to the frangible nature of the malleable component, etc.).

It is noted that these exemplary embodiments can be used in conjunction with the intracochlear stiffener, whether such stiffener be a malleable component or not, and also as a separate feature without utilization in conjunction with such intracochlear stiffeners. Again, any feature detailed herein can be used separately without any other feature detailed herein unless otherwise specified. In this vein, an exemplary embodiment includes the utilization of a removable stiffener in the intracochlear region without a stiffener located in the lead portion of the elongate lead assembly. With reference to FIG. 68, an exemplary embodiment includes an electrode array 125 that includes a removable stiffener 259, which electrode array is part of an elongate stimulating assembly, where there is no element 250 or any other stiffener component/malleable component located in the extra cochlear portion of the elongate stimulating assembly. Not that there are not embodiments that utilize the removable stiffener in conjunction with the stiffener located in the extra cochlear portion, as noted above, it is just that this embodiment utilizes such without the stiffener components in the extra cochlear portion (at least other than the portion of the removable stiffener that extends from the intracochlear portion into the extra cochlear portion). In an exemplary embodiment, the electrode array is a so-called straight electrode array. That is, when the removable stiffener is removed, and in the absence of all other forces on the electrode array, the natural state of the electrode array is to be substantially straight. This as opposed to a so-called curved electrode array, where at least when any removable stiffener is removed, and in the absence of all other forces on the electrode array, the natural state of the electrode array is to be at least somewhat curved.

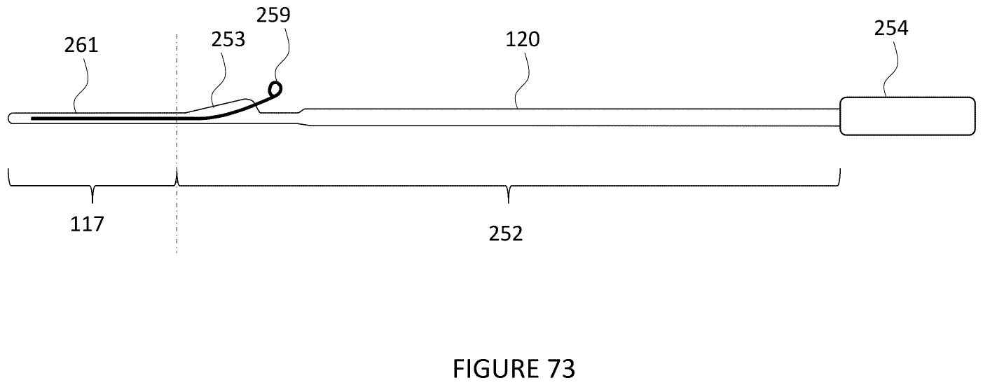

Accordingly, in an exemplary embodiment, there is a stimulation assembly of a cochlear implant, such as that depicted in FIG. 69 (where, in some embodiments, element 250 is not present, or any other stiffener for that matter, such as seen in FIG. 73 by way of example, comprising, a straight electrode array, including an intra-cochlear portion 117 including an array of electrodes (1-22, for example, but which can be more or less, according to the teachings detailed herein, and an extra-cochlear portion (the portion of the electrode array that is located in region 252)). In this exemplary embodiment, there is a removable stiffener (e.g., 259, located in the straight electrode array, the removable stiffener 259 extending from the extra cochlear portion to a location proximate a tip of the intra-cochlear portion (concomitant with the embodiment of FIG. 69 in this regard). Here, the removable stiffener is a removable stylet, concomitant with such stylets known in the art. Still, in some embodiments, the removable stiffener is an elastic component, such as a component made from Nitinol. In an exemplary embodiment, the stiffener has super elastic features, as noted above.

In an exemplary embodiment, the straight electrode array has no stiffener therein other than the removable stiffener.

It is noted that some features of the straight electrode array can have utilitarian value with respect to preserving so-called residual hearing. In this regard, in an exemplary embodiment, the embedded and/or removable stiffeners detailed herein that are located in the intracochlear region can be provided so as to obtain a so-called lateral wall placement of the electrode array and/or a so-called mid scala placement of the electrode array. In an exemplary embodiment, the stiffener can be sufficiently malleable so as to malleably deform as the electrode array is inserted into the cochlea due to forces applied by the lateral wall onto the electrode array as the electrode array extends further into the cochlea (owing to the curvature of the cochlear).

In the embodiments where the stiffener is a removable stylet, the stiffener can have any of the configurations detailed above with respect to the structure that is utilized to control or otherwise reduce or eliminate movement of the lead assembly due to the elasticity of that structure. In this regard, the teachings detailed above with respect to reducing movement or otherwise controlling movement of the lead assembly due to the elasticity of the lead assembly are also applicable to reducing movement or otherwise controlling movement of the electrode array due to the elasticity of the electrode array. In this regard, in an exemplary embodiment, the stiffener located in the electrode array, movable or otherwise, is configured to provide less stiffness than that which would correspond to traditional stylets, at least those approved for use by the FDA and/or the European Medicines Agency and/or the comparable agencies in the United Kingdom, Republic of France, the Federal Republic of Germany, Japan, the Republic of Korea, and/or the People's Republic of China. In an exemplary embodiment, the stiffness of the stiffener is no more than 90%, 85%, 80%, 75%, 70%, 65%, 60%, 55%, 50%, 45%, 40%, 35%, or 30% of that which is present with respect to aforementioned approved stylets, all other things being equal, at, for example, a mid-point of the stiffener, at a location ¼^{th} or ⅓^{rd} of the length (from either or both ends), on average, at 2, or 3, or 4, or 5, or more evenly spaced locations along the stiffener. That said, in some embodiments, the stiffener located in the intracochlear portion is designed to increase the elasticity of the electrode array relative to that which would be the case in the absence of the stiffener and/or relative to that which would be the case with conventional stylets. In an exemplary embodiment, the elasticity of the stiffener is more than 50%, 75%, 100%, 125%, 150%, 200%, 250%, 300%, 350%, 400%, 450%, 500%, 600%, 700%, 800%, 900%, or more than that which is present with respect to aforementioned approved stylets, all other things being equal, at, for example, a mid-point of the stiffener, at a location ¼^{th} or ⅓^{rd} of the length (from either or both ends), on average, at 2, or 3, or 4, or 5, or more evenly spaced locations along the stiffener.

In an exemplary embodiment, the stiffeners utilized in the embodiments detailed herein are made of annealed platinum, having a diameter of 100 to 250 micrometers over at least 50% of the length thereof, and at least over 60%, 65%, 70%, 75%, 80%, or 85%, or more or all of the length thereof. Palladium and/or gold can be utilized alternatively and/or instead of platinum. In some alternate embodiments, the stiffeners utilized in the embodiments detailed herein are substantially devoid, including completely devoid, of platinum, palladium and/or gold. In an exemplary embodiment, the stiffeners consist essentially of platinum, palladium and/or gold, while in other exemplary embodiments, the stiffeners consist essentially of materials other than platinum, palladium and/or gold.

It is noted that in some embodiments, the removable stiffeners can have rounded or bulbus tips to facilitate insertion into the lumen into which the stiffeners are placed so as to avoid damage to the body of the electrode array (e.g., the silicone forming the lumen in which the removable stiffener is located). This can also be the case with the stiffeners that are not removable as well.

It is noted that in at least some exemplary embodiments, the removable stiffeners can include locking components or securing components of the secure the removable stiffeners in place along a longitudinal position thereof. For example, the removable stiffeners can be configured such that graduated insertion depth can be predetermined and the removable stiffeners will be secured in place at those graduated insertion depths. It is also noted that other portions of the elongate stimulating assembly can include such locking features.

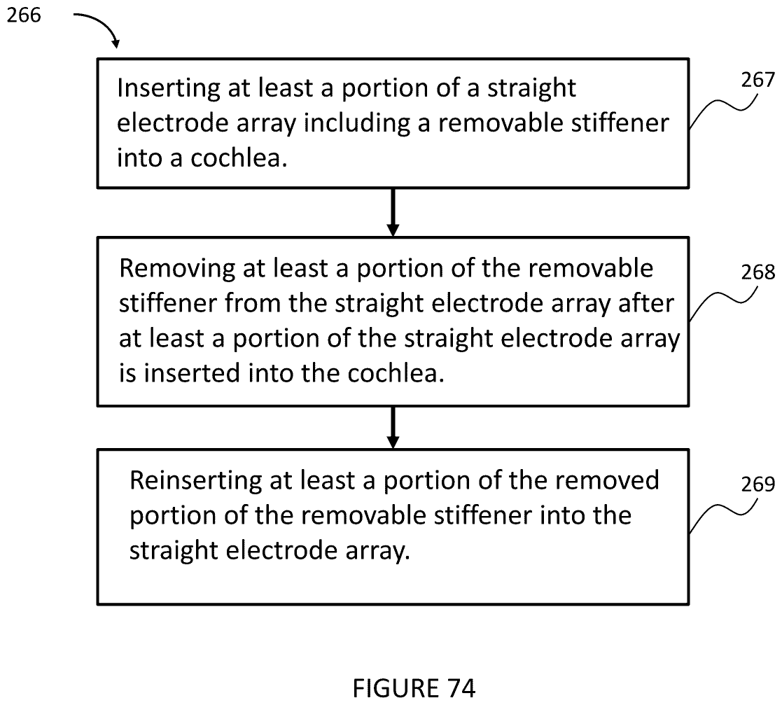

FIG. 74 presents an exemplary flowchart for an exemplary method, method 266, according to an exemplary embodiment. Method 266 includes method action 267, which includes inserting at least a portion of a straight electrode array including a removable stiffener, such as any of the electrode array's detailed herein and/or variations thereof in the form of a straight electrode array, into a cochlea of a recipient. In this embodiment, the straight electrode array is at least partially located in the electrode array such that a portion of the stiffener is also inserted into the cochlea (albeit such that the body of the electrode array is interposed between the stiffener and the environment inside the cochlea). Method 266 further includes method action 268, which includes removing at least a portion of the removable stiffener from the straight electrode array after at least a portion of the straight electrode array, which portion includes at least a portion of the stiffener, is inserted into the cochlea. That is, the portion that is removed is a portion that was already in the cochlea (again, albeit such that the body of the electrode array is interposed between the removable stiffener in the environment of the cochlea). In an exemplary embodiment, the removable stiffener is completely removed from the straight electrode array, while in other embodiments, only a portion of the removable stiffener is removed. In any event, method 266 further includes method action 269, which includes reinserting at least a portion of the removed portion of the removable stiffener into the straight electrode array. In the case where method action 268 corresponds to complete removal of the removable stiffener, method action 269 corresponds to inserting at least a portion of the removable stiffener back into the electrode array. This can also include reinserting the entire stiffener.

In an exemplary embodiment of the aforementioned methods, the stiffener bends during one of the aforementioned method actions. Indeed, in an exemplary embodiment, where the stiffener is an elastic component, the stiffener is maintained in the electrode array without retraction while at least 30%, 35%, 40%, 45%, 50%, 55%, 60%, 65%, 70%, 75%, 80%, 85%, 90%, 95%, or all of the longitudinal length of the intracochlear portion of the electrode array is inserted in the cochlea. In an exemplary embodiment, again where, for example, the stiffener is an elastic component, no more than 20%, 25%, 30%, 35%, 40%, 45%, 50%, 55%, 60%, 65%, 70%, 75%, 80%, 85%, or 90% by longitudinal length of the portion of the stiffener that is located in the electrode array and/or the portion of the stiffener that is inserted into the cochlea with the electrode array is removed at the point where one or more of the aforementioned percentages of the electrode array is inserted into the cochlea.

Note also that precedent method action 269, in an exemplary method, the method action of removing at least some or all of the straight electrode that was inserted into the cochlea is executed. That is, in an exemplary embodiment, or more specifically, in an exemplary scenario of insertion of the electrode array into the cochlea, a surgeon can execute method action 268, and then determine that the insertion regime is not proceeding according to that as desired or otherwise according to that which has more utilitarian value than that which is occurring. The surgeon can determine that he or she should remove part and/or all of the electrode array from the cochlea, which electrode array was previously inserted during this procedure. So as to achieve the stiffening characteristics corresponding to that which existed at the time that the portion of the electrode array that was removed from the cochlea, the surgeon or other healthcare professional reinserting at least a portion of the removable stiffener. This can be done while the removed portion of the electrode array is removed from the cochlea and/or while the removed portion is being removed from the cochlea.

While the focus of method 266 has been directed towards a straight electrode array, in some alternate embodiments, method 266 can be executed utilizing a curved electrode array.

An exemplary embodiment includes utilizing the removable stiffeners so as to vary in insertion depth of in a given electrode array over a relatively long period of time. For example, in an exemplary embodiment, at a first temporal period, the electrode array is inserted only a shallow insertion depth, such as 10 to 12 to 14 to 16 to 18 mm into the cochlea. In this regard, in this exemplary embodiment, the recipient has residual hearing at the lower and mid frequencies. Thus, the electrode array is inserted in the cochlea to stimulate the portions of the cochlea that are receptive to higher frequency sounds. In this exemplary embodiment, a removable stiffener is utilized during this insertion process, and subsequently completely removed from the electrode array. Alternatively, a removable stiffener is utilized, but the stiffener is retained in the electrode array, so as to provide stiffness in the portions of the electrode array located in the basal region. In any event, after some period of time, where the residual hearing of the recipient decreases, which could be one year, two years, three years, four years, five years or more after the initial insertion, and it is thus utilitarian to revise the electrode array positions to achieve a deeper insertion depth, a new removable stiffener is inserted into the electrode array while the electrode array is still inside the cochlea. This new removable stiffener can be partially or fully inserted into the electrode array. The surgeon or other healthcare professional then subsequently inserts the electrode array further into the cochlea, utilizing the new removable stiffener to provide stiffness to the electrode array in a conventional manner. In an exemplary embodiment, the surgeon or other healthcare professional subsequently inserts the electrode array into the cochlea a distance of 22 to 24 to 26 mm or more so as to achieve a full insertion depth thereof. Subsequent to full insertion, or during the full insertion process, the stylet can be removed leaving the un stiffened electrode array in the cochlea, just as was the case with the partially inserted electrode array during the first temporal period. Alternatively, in the in an exemplary embodiment, the removable stiffener is maintained in the electrode array after the partial insertion, and thus is implanted into the recipient, which removable stiffener can be locked in place according to the teachings detailed above and/or variations thereof. Subsequently, after the residual hearing is lost, and the electrode array is to be inserted further into the cochlea, the implanted removable stiffener can be utilized in the traditional manner during the full advancement of the electrode array, and then removed according to the traditional manner.

In at least some exemplary embodiments, the method of inserting the electrode array into the cochlea is such that the removable stiffener is removed from the electrode array as the electrode array is inserted into the cochlea, or, more accurately, the removable stiffener is removed from the electrode array as the portion of the electrode array reaches the basal turn of the cochlea so as to avoid contact with the wall of the cochlea and the portion of the electrode array that is stiffened by the removable stiffener. By way of example only and not by way of limitation, the electrode array can be inserted into the cochlea with the removable stiffener fully advanced into the electrode array, without moving the stiffener relative to the electrode array. However, as the electrode array reaches the first turn of the cochlea, or, more accurately, as the portion of the electrode array that is stiffened by the removable stiffener reaches the first turn the cochlea, the surgeon or other health care professional prevents the removable stiffener from advancing further into the cochlea. In an exemplary embodiment, the surgeon or other healthcare professional holds the stiffener in a static position relative to the anatomy of the recipient while the surgeon or other healthcare professional continues to advance the electrode array into the cochlea.

It is noted that in some exemplary embodiments, a portion of the electrode array that is stiffened by the removable stiffener can contact the lateral wall of the cochlea. In an exemplary embodiment, this can occur prior to reaching the first basal turn. In this regard, in at least some exemplary embodiments, there is no direct reactive force that reacts in the opposite direction of electrode advancement, contrary to that which exists when the electrode array reaches the first basal turn. In at least some exemplary embodiments, it is the contact of the lateral wall of the cochlea with a portion of the electrode array that is stiffened by the removable stiffener at and beyond the first basal turn that is avoided according to some exemplary method insertions, while contact of the lateral wall of the cochlea with a portion of the electrode array that is stiffened by the removable stiffener prior to the first basal turn is not avoided.

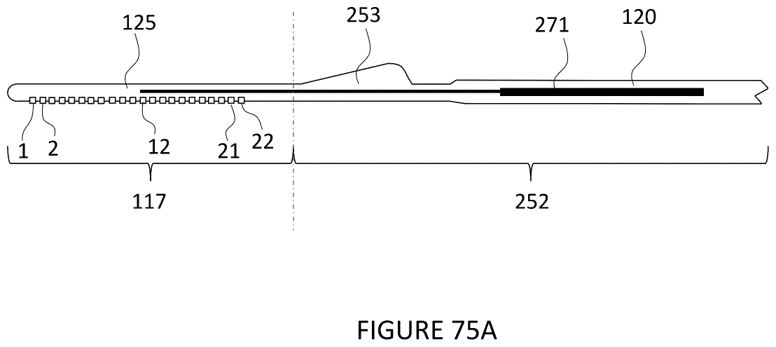

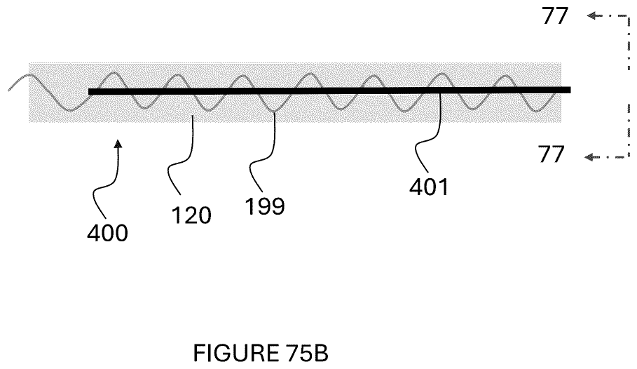

Briefly, it is noted that in at least some exemplary embodiments, a stiffness of the portion of the malleable component/stiffener that is part of the intra-cochlear portion is different than a stiffness of the portion of the malleable component that is part of the extra-cochlear portion. In some exemplary embodiments, this can be achieved by varying a diameter of the stiffener/malleable component. An exemplary embodiment of this is depicted in FIG. 75, where stiffener 271 has a first diameter within the intracochlear portion of the smaller than that of a second diameter of the stiffener 260 in the extra cochlear portion. Thus, in this exemplary embodiment, the portion of the stiffener/malleable component that is in the intracochlear portion is stiffer than the portion that is in the extra cochlear portion (all other things being equal). That said, in some alternative embodiments, this arrangement can be reversed, where the first diameter of the stiffener within the intracochlear portion is larger than that of the second diameter in the extra cochlear portion, and thus the portion that is in the extra cochlear portion of the stiffener portion in the intracochlear portion (all other things being equal). Note also that this concept can be applied to other locations along the extra cochlear portions of the various malleable component detailed herein and/or variations thereof.

In some alternate embodiments, a material stiffness of a portion of the stiffener/malleable component that is part of the intracochlear portion is different than a material stiffness of the portion of the stiffener/malleable component that is part of the extra-cochlear portion, wherein the stiffener/malleable component is a monolithic component. By “material stiffness,” it is meant that the stiffness of the material as a material property, as opposed to the stiffness that results from the structural geometry. That is, irrespective of the relative dimensions of the stiffener, when a per unit basis, the stiffness of the material is different. By way of example only and not by way of limitation, this can be achieved by tempering or heat-treating the two portions of the stiffener differently, such that one portion has a higher stiffness than the other portion. In an exemplary embodiment, one portion can be fully annealed (e.g. the portion in the intracochlear region or at least a portion thereof) in one portion can be partially annealed (e.g. the portion outside the intracochlear region, or at least a portion thereof). In an exemplary embodiment, the material stiffness of the portion that is located in the intracochlear region is greater than that which is the case in the extra cochlear portion while in other embodiments this is reversed. In an exemplary embodiment, the aforementioned material stiffnesses are different, where the stiffener is a monolithic component and the diameter of the stiffener is uniform between the two portions (the portion located in the intracochlear portion of the portion located in the extra cochlear portion).

Any device, system, and/or method that can enable the stiffener to have different stiffness is along the longitudinal length thereof can utilize in at least some exemplary embodiments.

In an exemplary embodiment, there is an elongate stimulation assembly of an implantable stimulation device, comprising an intra-cochlear portion including an array of electrodes; and an extra-cochlear portion extending from the intra-cochlear portion, wherein the extra-cochlear portion includes a malleable component extending in an elongate manner, a beginning of the malleable component extending from a location proximate the intra-cochlear portion (e.g., the beginning of the component being located at the beginning of the lead assembly (the distal beginning), or close thereto, and/or less than 1, 2, 3, 4, 5, 6, 7, 8, 9, 10, 11, 12, 13, 14, 15, 16, 17, 18, 19, 20 mm distally or proximally the distal beginning of the lead assembly or a distance from the end of the intracochlear portion) to a location in the extra-cochlear portion that is less than about XXX the length of the elongate stimulation assembly. In an exemplary embodiment, XXX is 90%, 85%, 80%, 75% (three-quarters), 70%, 66.66% (⅔rds), 65%, 60%, 55%, 50%, 45%, 40%, 35%, 33.333% (⅓^{rd}), 30%, 25%, 20%, 15%, or 10%.

It is noted that in an exemplary embodiment, the stylet/removable stiffener of the intra-cochlear portion, or any other stiffener removable or otherwise, can be tapered, or have generally varying cross-section, to have a varied bending stiffness along its length.

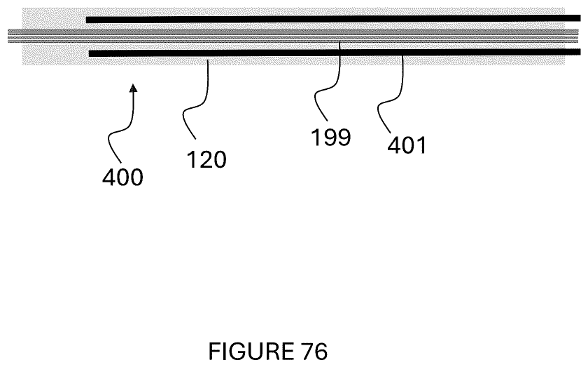

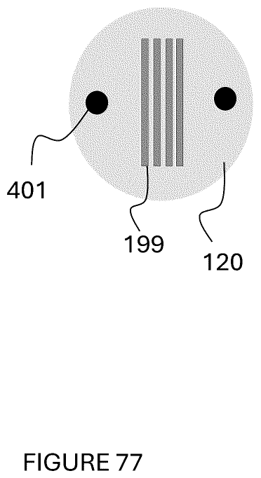

The concepts of using lead wires that are arrayed in a wavy and/or serpentine and/or zigzag pattern, etc., are not only applicable to the cage concepts detailed herein, but are also applicable to the malleable wires/components detailed herein. In this regard, FIG. 75B presents a conceptual schematic of a portion 400 of an elongate stimulating assembly when viewed from the side. Here, there are a plurality of electrical lead wires 199 (only one can be seen in the side view but more than one can be seen in the top view of the embodiment of FIG. 75B, which is presented in FIG. 76) embedded in a silicone body 120, concomitant with the teachings detailed above. Also seen is the malleable component 401, which can correspond to a malleable wire as detailed above (that of the embodiment of FIG. 8 for example). FIG. 76 shows a top view of the portion 400 of the elongate stimulating assembly, where it can be seen that there are 2 malleable components 401, one on either side of the lead wires 199. FIG. 76 shows how the lead wires 199 can be located in a staggered manner in the lateral directions, but can be aligned with each other as seen in FIG. 75B with respect to location in the longitudinal direction and the vertical direction. But note that this may not necessarily be the case, because the wires could be staggered and/or out of phase, etc., as detailed above. FIG. 77 shows a cross-sectional view of the portion 400 of the elongate stimulating assembly, where the malleable components 401 can be seen to flank the sides of the 4 lead wires 199 shown, all of which is embedded in a silicon body 120. This embodiment shows only 4 lead wires in the interest of clarity, in other embodiments, more lead wires or fewer lead wires can be utilized, consistent with the teachings detailed herein. Note that the various arrangements for the lead wires can be combined.

The embodiment of FIG. 76 shows the malleable components 401 at the same height. In some embodiments, this is not the case.

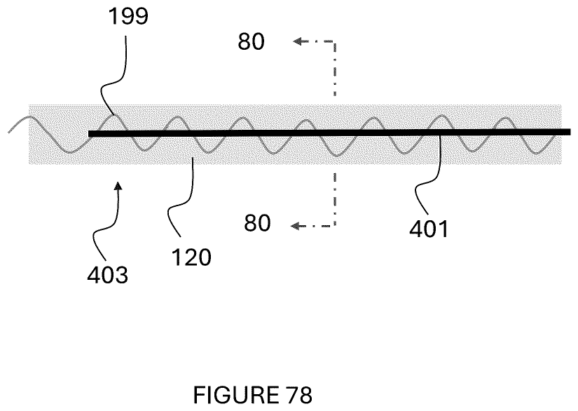

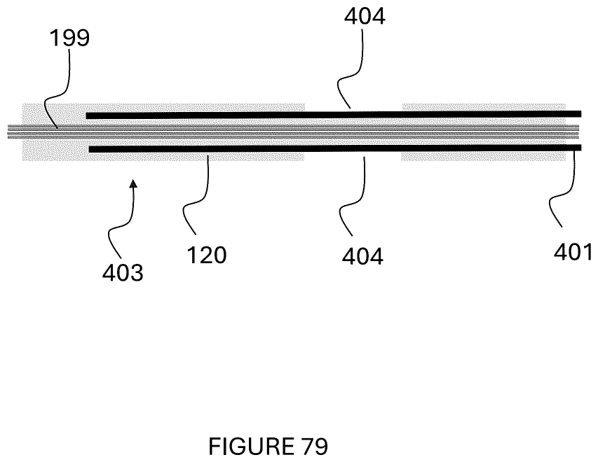

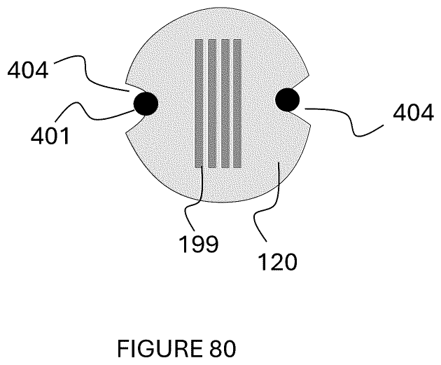

The concepts of using the component(s) that are used to maintain position of the elongate stimulation assembly as an electrical return electrode/electrical return interface are not only applicable to the cage concepts detailed herein, but are also applicable to the malleable wires/components detailed herein. FIG. 78 shows an exemplary portion 403 of an elongate stimulating assembly according to such an embodiment, as seen from the side. FIG. 79 shows a view of the portion 43 when viewed from the top. As seen, there are locations of the silicon body 120 that have been removed and/or were not present when the body 120 was formed about the malleable components 401 and the wires 199, thus leaving exposure areas 404. Thus, the conductive material of the malleable components 401 is exposed in the exposure areas 404 to the ambient environment of the elongate stimulating assembly. Thus, in an embodiment, this can enable the use of the exposed areas as a return, and electrical return, of the stimulating system which the stimulating assembly is a part. FIG. 80 shows the cross-sectional view of the portion 403, showing the exposure areas 404.

It is noted that while in some embodiments, the structures detailed herein are depicted as having a cross-section that is circular, other embodiments can utilize structures having cross-sections of different shapes, such as by way of example only and not by way of limitation, square shapes, diamond shapes, hexagonal shapes, etc., of cross-sections can be utilized. Further, while the structures detailed herein are depicted as having symmetrical cross-sections, in some alternative embodiments, the cross-sections are non-symmetric. By way of example only and not by way limitation, non-even rectangular shapes, oval shapes, pentagonal shapes and/or triangular cross-sections can be utilized. Any structure that can enable the teachings detailed herein and/or variations thereof to be practiced can be utilized in at least some exemplary embodiments.

It is noted that with respect to the helical structures detailed above, in at least some exemplary embodiments, the helical structures are wires formed into a helical shape.

It is noted that in at least some exemplary embodiments, the malleable structures detailed herein can be utilized to conduct electricity or otherwise conduct stimulation signals in a manner analogous to and/or the same as that which occurs with respect to the lead wires. In an exemplary embodiment, the malleable component establishes electrical communication with at least one electrode of the array of electrodes.

It is noted that in at least some exemplary embodiments, the teachings detailed herein can have utilitarian value with respect to reducing and/or eliminating a torque that is applied to the electrode array due to the lead assembly. Accordingly, an exemplary embodiment entails an implanted stimulating assembly that is relatively torque free with respect to the electrode array assembly while the electrode array assembly is located in the cochlea. Still further by way of example, in an exemplary embodiment, the teachings detailed herein can have utilitarian value with respect to reducing and/or eliminating electrode migration out of the cochlea and/or within the cochlea (e.g., twisting of the electrode array within the cochlea, movement of the electrode array out of the cochlea, at least in part, etc.). In at least some exemplary embodiments, the aforementioned teachings can result in fatigue failure relief/a reduction in failure due to fatigue of one or more portions of the stimulating assembly.

It is further noted that in at least some exemplary embodiments, the teachings detailed herein can have utilitarian value with respect to managing the growth of bone tissue with respect to children as they grow. That is, in an exemplary embodiment, because of the additional portion of the stimulating assembly located within a cavity formed by the mastoid cavity in the middle ear (the “slack”), growth of the mastoid bone which moves the receiver/stimulator away from the cochlea will not impart a stress on to the stimulating assembly that would pull the stimulating assembly out of the cochlea or otherwise impart a torque and/or a force onto the electrode array located in the cochlea.

As noted above, some and/or all of the teachings detailed herein can be used with a hearing prosthesis, such as a cochlear implant. That said, while the embodiments detailed herein have been directed towards cochlear implants, other embodiments can be directed towards application in other types of hearing prostheses, such as by way of example, other types of electrode arrays used in medical devices (e.g., pacemakers, nerve stimulators, etc.). Indeed, embodiments can be utilized with any type of medical device that utilizes an implanted electrode array, or even a non-implanted array, at least if there is utilitarian value with respect to conducting a test for an open circuit while the electrode array is located within packaging.

It is noted that any disclosure with respect to one or more embodiments detailed herein can be practiced in combination with any other disclosure with respect to one or more other embodiments detailed herein.

In an exemplary embodiment, there is an elongate stimulation assembly of an implantable stimulation device, comprising an intra-cochlear portion including an array of electrodes; and an extra-cochlear portion extending from the intra-cochlear portion, wherein the extra-cochlear portion includes a plurality of electrical lead wires in electrical communication with the array of electrodes and a malleable component extending in an elongate manner such that at least a portion of the malleable component is located further away from or the same distance from a longitudinal axis of the extra-cochlear portion than a portion of least one of the electrical leads of the plurality of electrical leads.

In an exemplary embodiment as described above and/or below, there is an elongate stimulation assembly as described above and/or below, wherein the malleable component is a metallic element. In an exemplary embodiment as described above and/or below, there is an elongate stimulation assembly as described above and/or below, wherein the extra-cochlear portion includes a lead body; and the malleable component is a metal wire embedded in the lead body. In an exemplary embodiment as described above and/or below, there is an elongate stimulation assembly as described above and/or below, wherein: the extra-cochlear portion includes a lead body; and the malleable component is a metal wire located completely outside the lead body. In an exemplary embodiment as described above and/or below, there is an elongate stimulation assembly as described above and/or below, wherein: the extra-cochlear portion includes a lead body in which are located the plurality of lead wires; and the malleable component establishes electrical communication with at least one electrode of the array of electrodes. In an exemplary embodiment as described above and/or below, there is an elongate stimulation assembly as described above and/or below, wherein: the malleable component is a helical structure wrapped around at least some of the lead wires. In an exemplary embodiment as described above and/or below, there is an elongate stimulation assembly as described above and/or below, wherein: the extra-cochlear portion includes a lead body; the lead wires are encapsulated in the lead body; and the malleable component is a helical structure extending about the lead body at least partially external to the lead body. In an exemplary embodiment as described above and/or below, there is an elongate stimulation assembly as described above and/or below, wherein: the malleable component is a helical structure wrapped around all of the lead wires.

In an exemplary embodiment, there is an elongate stimulation assembly of a cochlear implant, comprising: an intra-cochlear portion including an array of electrodes; lead wires extending from the intra-cochlear region in electrical communication with the array of electrodes, the lead wires being located in an elongate lead body; and a malleable component extending in an elongate manner at least partially along with the lead wires, wherein the malleable component is located closer to an outer surface of the lead body than at least one of the lead wires or wherein the malleable component is located the same distance from the outer surface of the lead body as at least one of the lead wires. In an exemplary embodiment, there is an elongate stimulation assembly of a cochlear implant as described above and/or below, wherein: the malleable component is a helical structure. In an exemplary embodiment, there is an elongate stimulation assembly of a cochlear implant as described above and/or below, wherein: the lead wires are encapsulated in the lead body; and the malleable component is a helical structure extending about the lead body attached to a surface of the lead body. In an exemplary embodiment, there is an elongate stimulation assembly of a cochlear implant as described above and/or below, wherein: the lead wires are encapsulated in the lead body; and the malleable component is a helical structure extending about the lead body such that the lead body can move locally relative to the helical structure. In an exemplary embodiment, there is an elongate stimulation assembly of a cochlear implant as described above and/or below, wherein: the lead wires are encapsulated in a lead body; and the malleable component is a helical structure extending about the lead body such that the lead body can move locally relative to the helical structure while preventing global movement of the lead body. In an exemplary embodiment, there is an elongate stimulation assembly of a cochlear implant as described above and/or below, wherein: the lead wires are encapsulated in a lead body; and the malleable component is attached to an outer surface of the lead body. In an exemplary embodiment, there is an elongate stimulation assembly of a cochlear implant as described above and/or below, wherein: the malleable component forms one helix of a double helix, and the lead wires form the other helix of the double helix.

In an exemplary embodiment, there is a method, comprising: obtaining access to a subcutaneous region of a recipient's head; implanting a stimulating assembly at the subcutaneous region, wherein the action of implanting the electrode assembly includes plastically deforming a first portion of the stimulating assembly so as to maintain the first portion now deformed at a first orientation due to the deformation of the first portion. In an exemplary embodiment, there is a method as described above and/or below, further comprising: inserting at least a portion of an electrode array into a cochlea, wherein the action of deforming the first portion of the stimulating assembly is executed before insertion of the at least a portion of the electrode array into the cochlea. In an exemplary embodiment, there is a method as described above and/or below, wherein: the action of implanting the stimulating assembly includes plastically deforming a second portion of the stimulating assembly so as to maintain the second portion now deformed at a second orientation due to the deformation of the second portion. In an exemplary embodiment, there is a method as described above and/or below, further comprising: inserting at least a portion of an electrode array into a cochlea, wherein the action of deforming the first portion of the stimulating assembly is executed before insertion of the at least a portion of the electrode array into the cochlea, and the action of deforming the second portion of the stimulating assembly is executed after insertion of the at least a portion of the electrode array into the cochlea. In an exemplary embodiment, there is a method as described above and/or below, wherein: the accessed subcutaneous region includes an artificial channel in a mastoid bone of the recipient leading to a mastoid cavity, wherein the mastoid cavity is part of a cavity that is bounded in part by a round and oval window of a cochlea of the recipient; the action of implanting the electrode assembly includes placing the first portion of the stimulating assembly into the artificial channel such that a first sub-portion is located in the channel and a second sub-portion extends from the channel into the mastoid cavity; and the action of deforming the first portion entails bending the first portion such that the second sub-portion is moved from a first orientation relative to the first sub-portion to a second orientation relative to the first sub-portion, and the plastic deformation maintains the second sub-portion at the second orientation.

In an exemplary embodiment, there is a method as described above and/or below, wherein: the second orientation is such that a longitudinal axis of the second sub-portion is at least about 45 degrees from a longitudinal axis of the first sub-portion. In an exemplary embodiment, there is a method as described above and/or below, wherein: the subcutaneous region includes a mastoid cavity; and the method is executed without placing any portion of the stimulating assembly against any bone overhanging the mastoid cavity. Wherein the action of implanting the electrode assembly further includes: inserting an intracochlear electrode into a cochlea through an opening therein; and securing a portion of the electrode assembly located between the intracochlear electrode and a receiver/stimulator connected to the stimulating assembly in a mastoid cavity without contacting a bony overhang of the mastoid cavity.

In an exemplary embodiment, there is an elongate stimulation assembly of an implantable stimulation device, comprising: an intra-cochlear portion including an array of electrodes; and an extra-cochlear portion extending from the intra-cochlear portion, wherein a malleable component extending from the extra-cochlear portion to the intra-cochlear portion such that the extra-cochlear portion and the intra-cochlear portion also include respective portions of the malleable component. In an exemplary embodiment, there is an elongate stimulation assembly of an implantable stimulation device as described above and/or below, wherein: the array of electrodes includes at least 10 electrodes arrayed along a longitudinal direction of the intra-cochlear portion; and the malleable component extends from the extra-cochlear portion into the intra-cochlear portion such that the malleable component extends past at least 5 of the electrodes of the electrode array. In an exemplary embodiment, there is an elongate stimulation assembly of an implantable stimulation device as described above and/or below, wherein: the malleable component does not extend past more than ⅔rds of the electrodes of the array of electrodes. In an exemplary embodiment, there is an elongate stimulation assembly of an implantable stimulation device as described above and/or below, wherein: the malleable component extends at least one of past all of the electrodes or past 90% of the electrodes. In an exemplary embodiment, there is an elongate stimulation assembly of an implantable stimulation device as described above and/or below, wherein: the malleable component extends from the intra-cochlear portion to a location at least proximate a housing containing a stimulator of the implantable stimulating device, or the malleable component extends from the intra-cochlear portion to the housing. In an exemplary embodiment, there is an elongate stimulation assembly of an implantable stimulation device as described above and/or below, wherein: the malleable component extends at least substantially the entire length of the extra-cochlear portion and substantially the entire length of the intra-cochlear portion. In an exemplary embodiment, there is an elongate stimulation assembly of an implantable stimulation device as described above and/or below, wherein: the malleable component extends at least substantially the entire length of the extra-cochlear portion and no more than about 80% of the intra-cochlear portion. In an exemplary embodiment, there is an elongate stimulation assembly of an implantable stimulation device as described above and/or below, wherein: the malleable component is a monolithic component. In an exemplary embodiment, there is an elongate stimulation assembly of an implantable stimulation device as described above and/or below, wherein: a material stiffness of the portion of the malleable component that is part of the intra-cochlear portion is different than a material stiffness of the portion of the malleable component that is part of the extra-cochlear portion, wherein the malleable component is a monolithic component. In an exemplary embodiment, there is an elongate stimulation assembly of an implantable stimulation device as described above and/or below, wherein: a material stiffness of the portion of the malleable component that is part of the intra-cochlear portion is different than a material stiffness of the portion of the malleable component that is part of the extra-cochlear portion, wherein the malleable component is a monolithic component and a diameter of the malleable component is uniform between the two portions.

In an exemplary embodiment, there is an elongate stimulation assembly of an implantable stimulation device, comprising: an intra-cochlear portion including an array of electrodes; and an extra-cochlear portion extending from the intra-cochlear portion, wherein a first malleable component is located in the extra-cochlear portion; and a stiffener component is located in the intra-cochlear portion, the stiffener component being separate from the first malleable component. In an exemplary embodiment, there is an elongate stimulation assembly as described above and/or below, wherein: the stiffener component is a second malleable component separate from the first malleable component. In an exemplary embodiment, there is an elongate stimulation assembly as described above and/or below, wherein: the stiffener component has a first portion located in the intra-cochlear portion and a second portion located in the extra cochlear portion. In an exemplary embodiment, there is an elongate stimulation assembly as described above and/or below, wherein: the first malleable component does not extend into the intra-cochlear portion. In an exemplary embodiment, there is an elongate stimulation assembly as described above and/or below, wherein: the first malleable component overlaps with the stiffener component in a longitudinal direction of the elongate stimulation assembly. In an exemplary embodiment, there is an elongate stimulation assembly as described above and/or below, wherein: the stiffener component is a removable stylet. In an exemplary embodiment, there is an elongate stimulation assembly as described above and/or below, further comprising: a second malleable component that is separate from the first malleable component and separate from the stiffener. In an exemplary embodiment, there is an elongate stimulation assembly as described above and/or below, wherein: the second malleable component is longitudinally spaced away from the first malleable component. In an exemplary embodiment, there is an elongate stimulation assembly as described above and/or below, wherein: the first malleable component is the only malleable component that is completely outside the intra-cochlear portion. In an exemplary embodiment, there is an elongate stimulation assembly as described above and/or below, wherein: the stiffener component is an elastic component that extends at least about half the length of the intra-cochlear portion. In an exemplary embodiment, there is an elongate stimulation assembly as described above and/or below, wherein the first malleable component is a removable component. In an exemplary embodiment, there is an elongate stimulation assembly as described above and/or below, wherein: the first malleable component is configured to have a stiffness that is adjustable at a local portion thereof. In an exemplary embodiment, there is an elongate stimulation assembly as described above and/or below, wherein: the extra-cochlear portion is configured to have a stiffness that is adjustable at a local portion thereof.

It is noted that some embodiments include a method of utilizing the apparatuses and systems having one or more or all of the teachings detailed herein and/or variations thereof. In this regard, it is noted that any disclosure of a device and/or system herein also corresponds to a disclosure of utilizing the device and/or system detailed herein, at least in a manner to exploit the functionality thereof. Further, it is noted that any disclosure of a method of manufacturing corresponds to a disclosure of a device and/or system resulting from that method of manufacturing. It is also noted that any disclosure of a device and/or system herein corresponds to a disclosure of manufacturing that device and/or system. Moreover, any disclosure of a method action herein also corresponds to a system and/or a device for executing that method action. Also, any disclosure of a device of system herein corresponds to a disclosure of a method of using that device and/or system, and a method of manipulating that device and/or system using the features disclosed herein.

While various embodiments have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

What is claimed is:

1. An elongate stimulation assembly of an implantable stimulation device, comprising:
an intra-cavity portion including a plurality of electrodes; and
an extra-cavity portion extending from the intra-cavity portion, wherein
the extra-cavity portion includes a plurality of electrical lead wires in electrical communication with the plurality of electrodes and a malleable component extending in an elongate manner such that at least a portion of the malleable component is located further away from or the same distance from a longitudinal axis of the extra-cavity portion than a portion of least one of the electrical leads of the plurality of electrical leads.

2. The assembly of claim 1, wherein:
the extra-cavity portion includes a lead body;
the malleable component is a metal wire embedded in the lead body;
the assembly is an elongate retinal implant stimulation assembly; and
the cavity is a cavity in an eye of a human.

3. The assembly of claim 1, wherein:
the extra-cavity portion includes a lead body; and
the malleable component is a metal wire located completely outside the lead body.

4. The assembly of claim 1, wherein:
the extra-cavity portion includes a lead body in which are located the plurality of lead wires; and
the malleable component establishes electrical communication with at least one electrode of the array of electrodes.

5. The assembly of claim 1, wherein:
the malleable component is a helical structure wrapped around at least some of the lead wires.

6. The assembly of claim 1, wherein:
the extra-cavity portion includes a lead body;
the lead wires are encapsulated in the lead body; and
the malleable component is a helical structure extending about the lead body at least partially external to the lead body.

7. The assembly of claim 1, wherein:
the malleable component is a helical structure wrapped around all of the lead wires.

8. An elongate stimulation assembly of an implantable stimulation device, comprising:
a first portion including a plurality of electrodes;
lead wires extending from the first portion in electrical communication with the plurality of electrodes, the lead wires being located in an elongate lead body; and
a cage component extending in an elongate manner at least partially along with the lead wires, wherein the lead wires extend within the cage component.

9. The assembly of claim 8, wherein:
the cage component is located on an outside of the assembly.

10. The assembly of claim 8, wherein:
the cage component is underneath silicone material.

11. The assembly of claim 8, wherein:
the cage component is kink proofed.

12. The assembly of claim 8, further comprising:
a malleable component that forms a helix.

13. The assembly of claim 8, wherein:
the cage component includes portions configured to more easily plastically deform than other portions of the cage component.

14. The assembly of claim 8, wherein:
an extrapolated volume of the cage component is made up of less than 20% of solid material.

15. A device, comprising:
a stimulating assembly of an implantable stimulating device, including a lead assembly made at least partially of a structure having portions that are more susceptible to plastic deformation than other portions of the structure, wherein
the device is configured to resist movement of at least a portion of the lead assembly, the movement of the lead assembly being due to the plastic deformation.

16. The device of claim 15, wherein:
the structure is configured to provide hinge points that undergo larger strains during bending of the structure than other portions adjacent the hinge points, which larger strains produced the plastic deformation.

17. The device of claim 15, wherein:
the structure is a stent structure.

18. The device of claim 15, wherein:
with respect to location along a longitudinal axis of the structure, the structure has at least five (5) first cross-sections normal to the longitudinal axis and at least five (5) second cross-sections normal to the longitudinal axis respectively located in an alternating manner along the longitudinal axis and equidistant from each other with respect to location along the longitudinal axis; and
the first cross-sections have at least 75% less material of the structure than the second cross-sections by area.

19. The device of claim 15, wherein:
the structure is encapsulated in an overmoulded silicone body.

20. The device of claim 15, wherein:
the structure has a first section extending along a longitudinal axis of the structure that extends about the longitudinal axis in a zig-zag manner with respect to axial direction.

21. The device of claim 15, wherein:
the structure has a first section extending along a longitudinal axis of the structure that extends about the longitudinal axis in an arcuate manner with respect to radial direction.

22. The assembly of claim 8, wherein:
the assembly includes a plurality of electrodes; and
the cage component is in electrical communication with at least one of the electrodes of the plurality of electrodes.

23. The assembly of claim 8, wherein:
the assembly includes a plurality of stimulating electrodes; and
the cage establishes an electrical return for a circuit that includes the assembly.

24. The assembly of claim 8, wherein:
the assembly includes a plurality of stimulating electrodes;
the cage component is part of a cage apparatus that includes a non-cage portion; and
the non-cage portion establishes an electrical return for a circuit that includes the assembly, the non-cage portion being an interface to an ambient environment of the assembly, and the cage being in electrical conductivity with the non-cage portion but more isolated from the ambient environment of the assembly than the non-cage portion, the cage component functioning as a return conductor from the non-cage portion.

25. The assembly of claim 24, wherein:
all of the cage component is covered with an electrically insulative material; and
at least a portion of the non-cage portion is not covered with an electrically insulative material.

26. The assembly of claim 24, wherein:
the non-cage portion is a hollow cylindrical body; and
the cage component extends away from at least one side of the hollow cylindrical body and is in contact with the hollow cylindrical body.

27. The assembly of claim 24, wherein:
the non-cage portion is a hollow cylindrical body that envelopes a portion of the cage component and the interior of the hollow cylindrical body is in direct contact with an exterior of the cage component.

28. The assembly of claim 24, wherein:
the non-cage portion is a body having an outer surface area of solid material that is at least three times that of an average outer surface area of solid material of the cage component.

29. The assembly of claim 23, wherein:
a portion of the cage component is covered with an electrically insulative material; and
a portion of the cage component is exposed to an ambient environment, thereby establishing the electrical return.

30. The assembly of claim 8, further comprising:
a second cage component extending in an elongate manner partially along with the lead wires, wherein the lead wires extend within the second cage, wherein
the cage component extends partially along with the lead wires and is spaced away from the second cage component in the longitudinal direction, and
the cage component is electrically isolated from the second cage component.

31. The assembly of claim 30, wherein:
the cage component establishes a first electrical return electrode; and
the second cage component establishes a second electrical return electrode.

32. The assembly of claim 31, wherein:
the second cage component is electrically connected to electronics of a receiver-stimulator to which the assembly is electrically and physically attached.

33. A method, comprising:
obtaining an implantable cage apparatus;
moulding silicone so that the cage apparatus is encapsulated in an overmoulded silicone tube, thus resulting in a silicone tube-cage assembly; and
inserting the silicone tube-cage assembly over a plurality of lead wires of a cochlear implant electrode array and/or inserting the plurality of lead wires into the silicone tube-cage assembly.

34. The method of claim 33, further comprising:
prior to inserting the silicone tube-cage assembly over the lead wires/inserting the lead wires into the silicone tube-cage assembly, expanding the silicone tube-cage assembly in a radial direction.

35. The method of claim 33, further comprising:
after inserting the silicone tube-cage assembly over the lead wires/inserting the lead wires into the silicone tube-cage assembly, contracting the silicone tube-cage assembly in the radial direction.

36. The method of claim 33, wherein:
the action of contracting the silicone tube-cage assembly is executed by at least one of inflation of an inflatable cuff or heatshrink.

37. The method of claim 35, wherein:
the action of contracting the silicone tube-cage assembly plastically deforms the silicone tube-cage assembly.

38. The method of claim 35, wherein:
the plurality of lead wires are arranged in a wavy lead configuration.

39. The method of claim 29, further comprising:
attaching contracted silicone tube-cage assembly to a cochlear implant receiver-stimulator.
